(19) United States
(12) Patent Application Publication
(10) Pub. No.: US 2005/0216097 A1
(43) Pub. Date: Sep. 29, 2005

(54) Tensegrity joints for prosthetic, orthotic, and robotic devices

(76) Inventor: Jerome Rifkin, Louisville, CO (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/080,972

(22) Filed: Mar. 16, 2005

Related U.S. Application Data
(60) Provisional application No. 60/553,619, filed on Mar. 16, 2004.

(51) Int. Cl. A61F 2/66

(52) U.S. Cl. 623/53; 623/55

(57) ABSTRACT

Embodments of the invention relate to a prosthetic, orthotic, or robotic foot having at least two joints. One joint is located in a position analogous to the human MTP joint, and the other is located in a position analogous to the human subtalar joint. Motions of these two joints are mechanically couples. Furthermore, these joints are created using “tensegrity” design principals, where connections between the compression members are made by a network of tension members. These tension members create axes of motion, and limitations on those axes of motion. Actuators or linear elastic “springs” are use to alter the torque/angular deflection response curve of these joints, so that the rollover profile of the human foot can be duplicated by this invention.

### CROSS-REFERENCE TO RELATED APPLICATIONS

The invention claims the benefit of priority of provisional U.S. Patent Application No. 60/553,619, filed Mar. 16, 2004, the entirety of which is incorporated herein by reference.

### DESCRIPTION OF THE INVENTION

1. Field of the Invention

Embodiments of the present invention relate to a prosthetic, orthotic, or robotic foot that simulates the coordinated motions of the natural human foot in walking gait. More particularly, embodiments of the present invention relate to a prosthetic, orthotic, or robotic foot having three segments connected by two joints: one joint analogous to the human first metatarsophalangeal joint, and the other joint analogous to the human subtalar joint. The three segments correspond to a toe, a forefoot, and a heel.

2. Background of the Invention

People who lose a leg today may be in a bad situation. Some days, a simple staircase may seem like an insurmountable challenge. Walking up a grassy slope is too difficult to attempt, because multiple falls may be inevitable. War, accidents and disease keep this disadvantaged population growing. Prosthetics, or synthetic replacements for missing anatomical structures, hold the promise of restoring some of this lost function and improving quality of life.

Just trying to regain functional mobility, amputees spend an average of $8,000 on below-knee (BK) prosthetic legs that last three to five years. Rather than spend this money on costly, non-repairable devices, one hundred and twenty thousand American amputees have chosen crutches or wheelchairs, and they won't walk again.

Just as the speed of a vehicle is maintained through regular energetic pushes received from pistons firing in the engine, normal human gait relies on well-timed pushes from the anatomy of the foot, during the toe-off portion of the walking cycle. It is precisely this “timing of toe-off”, while providing a stable, level base—a preferable innovation addressed in this application—that existing feet prostheses lack and that may be relevant for natural and comfortable ambulation.

The human gait is in reality a very complex process that at a basic level may be described as a series of repeating operations carried out by a single leg: 1) initial heel strike, 2) double support as both feet contact the ground, 3) stance phase as one leg supports the entire body weight, 4) pre-swing or heel-rise as the heel rises from the ground, 5) toe-off as the moment that the toes lose contact with the ground, and finally 6) swing phase, where the leg, acting as a pendulum, comes forward in preparation to repeat the process. In a two legged description of pre-swing, the heel of the contralateral leg strikes the ground at the exact moment that the ipsilateral heel rises. This is called double stance phase, and may be relevant to understanding the innovations presented in this application. Coordinated movement between the legs and the overall balance and trajectory of the body dynamic may be also relevant to successful ambulation.

Currently, there are two dominant paradigms of prosthetic foot design: post-like, conventional feet (CF) and leaf-spring-like, energy storing feet (ESF). Both of these designs change shape under loading, in an attempt to mimic the human foot. The classic CF foot, also known as the Solid Ankle Cushioned Heel (SACH), foot may provide a stable base for support, and is functionally unchanged since its conception in the 1960's. Introduced in the 1980's, carbon-fiber, leaf-spring ESF designs allow amputees to run by mimicking the ankle plantar flexors, returning energy to their stride. Para-lympic records rivaling their Olympic counterparts show that the ESF paradigm works very well for running, but studies have failed to show that these benefits extend to walking. 40% of transtibial amputees do not use prostheses and 78% of transfemoral amputees forego this intervention. Thus, over 120,000 amputees do not use prosthetic legs, preferring wheelchairs or crutches, never walking again. Studies of amputee psychosocial adjustment have linked positive emotional coping and higher levels of physical independence.

Depending on the type of foot used, CF or ESF, and the specific manufacturer, there have been subtle but significant differences in parameters such as stride length, symmetry of stride, and timing of the various phases of gait. For either foot type, stride length is shorter for strides where the prosthesis is the supporting limb, gait symmetry is markedly decreased, and the timing of the phases of gait may be disrupted. Most notably, there is a shortened stance phase on the prosthesis, a late toe off, and a longer swing phase on the affected side as well. Studies also describe an early incidence of low back and patellar-femoral osteoarthritis in unilateral amputees. The literature clearly shows that current prostheses fail to walk like an intact limb. In fact, clinical prosthetists have expressed the opinion that some “middle ground” between the unsophisticated CF feet and the highly athletic ESF feet is needed. Embodiments of the invention outlined here may be just that middle ground.

To lay the foundation for the rest of this submission, a few questions may be asked. Precisely how may an intact limb walk? And what is the role of the foot in this process? To address the first question, this application may present two different types of engineering control systems, and may provide illustrative examples. To address the second question, more studies may be presented, furthering the discussion, showing results of highly detailed, instrumented gait studies of the foot. Comparisons between the functional movements of the human foot, and the functional movements of current prostheses may follow. Finally, this section may discuss the effect of these functional movements on energy consumption during walking, via oxygen consumption (VO2) analysis. The improvements embodied in embodiments of the proposed device may address many of the shortcomings seen in the current technology.

With all of the myriad muscles and bones in human hips, legs, and feet, there is no “right” answer for how to propel one's self across a room or up a slope; however, there may be more optimal solutions, for example, ones that may be less abusive to the anatomy and/or ones with more optimal energetic efficiency. Early incidence of osteoarthritis, a degenerative joint change, is one indicator of a suboptimal movement strategy.

There may be many ways to walk, and data shows that people don't walk in exactly the same way with each stride. The hips may work harder on some strides than others; sometimes the lower leg may contribute varying amounts torque to the stride. Walking from one's hips may be described as a “top-down” control mechanism, where forces from the proximal leg may dictate the position and accelerations of the distal structures. This mechanism is very clearly illustrated in above knee (AK) amputees. Until recent, expensive innovation of computer controlled knees, AK amputees who wanted to walk faster than the return rate of their knee spring had to use a “hip snap,” flinging their prosthesis out quickly with their hip flexors, and then quickly contracting their hip extensors to snap the prosthetic knee straight in time for heel-strike. Thus, the anatomic ranges of motion guided the position of the prosthetic anatomy, but the timing the movement was controlled by the hip, in a “top-down” fashion.

A “top-down” control mechanism may also be seen in studies of trans-tibial amputees. The iEMG data of one study showed a greater use of the biceps femoris (BF) as compared to the antagonistic vastus medialus (VM) in the amputated limb, as opposed to the normal limb. The mean ratios of BF/VM activity during the first half of stance phase was 3.8 in the amputated limb and 2.0 in the sound leg, with a P value of less than 0.042. Furthering elaboration on the “top-down” nature of this control system, an exceptionally statistically rigorous study from 2002 revealed some interesting trends in the flexor/extensor ratios for the knees of unilateral, trans-tibial amputees, as compared to normal volunteers. Though the amputees were much weaker than the normal control group, this study showed that there was no significant difference between the knee flexor/extensor ratios for peak bending moment, total work, or maximum power comparing either leg of the amputees and either leg of the non-amputees. Of course, the BF and VM may be also knee flexors and extensors, but not during the relevant time-span cited by the first study, early stance phase. Considering these studies together, one may conclude that trans-tibial amputees use the hip of the amputated leg more than the hip of their sound leg, and that they use their knee flexors and extensors normally. Clearly, the control mechanism being employed in a trans-tibially amputated limb is “top-down.”

The overuse of a particular muscle must result in overuse of the surrounding and supporting muscles. For example over loading a hip muscle causes the hip stabilizers to be over-recruited. If multifidus and transversus abdominus, the deepest pelvic stabilizers, may be overwhelmed, the larger quadratus lomborum (OL) and erector spinae (ES) muscles that may be normally used for motion may be recruited to help it. When the QL and ES are used as stabilizers, the agonists may also be recruited as stabilizers, just as transverses abdominus is recruited along with multifidus. When the QL and ES become a routine part of the stabilizing muscle pattern, they become tonic and rigid. Thus, putting a great deal of compression on the spine. This is a well known pattern of muscle use and, if allowed to progress unchecked, may eventually result in degenerative joint changes in the lower spine.

Walking from the foot, as opposed to the hip, may be modeled as a “bottom-up” control scheme, where the distal anatomy directs the position of the proximal anatomy. The coordination of the metatarsophalangeal joint (MTP) of the great toe and the subtalar joint may create a dynamic in gait where the proximal foot and tibia subtly change angular position. This angular change may be the start of building momentum for toe off. In context of the gait cycle, starting from single stance phase, as the tibial shaft moves past perpendicular and over the foot, the subtalar joint may be eccentrically loaded. This may be seen as a “flatter” transverse arch. This subtle motion may progress with the tibial shaft advancement, with a maximum angular change of 10 degrees. In double stance phase, much of this weight may be off-loaded to the other leg, but the transverse arch may not yet spring back into shape. In fact, this new conformation may be maintained until just after heel rise. When the heel leaves the ground, passing the remaining force loading to the ball of the great toe, the MTP of the great toe may be forced into extension. This motion may pull on the plantar aponeurosis, which in turn may pull on the calmayeus and the Achilles tendon. This action may loft the transverse arch back to its stance phase conformation, subtly altering the position of the ankle and the tibia, and Thus, may change position of the knee and hip. Graphs of the relative joint motion have been provided as FIG. 57 for reviewers who wish to see a graphical description.

The relevant anatomy for this coordination of the first MTP and subtalar joints is well documented. The plantar aponeurosis spans both joints, as may the tendon of the flexor hallucis longus. Different research references attribute this coordination to each of these sources. The action of arching the subtalar joint by forcibly extending the first MTP has been described as the Windlass mechanism, as shown in FIG. 56, and this passive, non-muscular change may be a function of timing and anatomic length. This timing may be influenced by the peronii, the tibialis anterior, and the intrinsic foot muscles. Of course, a passive prosthesis may not duplicate the action of these muscles, but it may mimic the action of the plantar aponeurosis. Due to the quasi-psuedoviscoelastic nature of the plantar aponeurosis and the surrounding musculature, this quick lofting of the plantar arch may be an energy storage mechanism. The energy may then be released, a moment later, on toe off. As seen in the temporal gait asymmetry of amputees, most notably in late stance and swing phases, studies have shown conclusively that this action is not accomplished in either CF or ESF designs.

These two distinct “ways of walking” represent extremes, and, as human nature dictates, we all walk with a varying degree of each mechanism. Amputees must rely exclusively on the strategy of top-down control, resulting in an overcompensation of the remaining anatomy which in turn may cause early degenerative changes. What is needed is a prosthesis that accurately imitates the relevant biomechanics of the natural foot, allowing for the contributions of the more efficient “bottom-up” gait style.

There is a definite coordination between the joints of the foot. For example, see FIGS. 56 and 57. The angular relationship shown between the forefoot (FF) and hallux (Hx) may be the angular position of the 1st MTP. The angular motion between the FF and hindfoot (HF) may reflect the motion of the subtalar joint. A few studies have explored the detailed biomechanics of the foot using this powerful analytical technique, but they did not combine the detailed foot analysis with the protocol for the rest of the body. Thus, no quantified joint powers were generated. Experts may also be aware of the subtle, but highly significant errors in instrumented gait analysis of ESF prosthesis gait. Failure to accurately model the center of curvature of the leaf spring foot, for the purpose of reverse engineering the joint torques, may be the documented source of this error. The standard seven segment lower body model, used to reverse engineer joint torques, may use a rigid single segment foot. This simplified model may leave out both the first MTP and the subtalar joints, masking the relevant contribution of the Windlass mechanism, a subtle “bottom-up” contributor of gait mechanics. Theoretically, a nine segment lower body model, as seen in computer simulations, may show sensitivity to changes in spring stiffness of the MTP joint at push off, but still may exclude the subtalar joint or any coordination of the two.

The movement of the subtalar joint and first MTP during stance phase and toe-off, as described in the previous section, may correlate to a relatively new area of prosthetics research. Roll-over shape may be defined as the geometry a foot/ankle complex takes during the single limb stance phase of walking. As the center of weight may pass over the long axis of the prosthetic foot, it may bend according to its stiffness. The shape described by this bending may be the rollover-shape, and it may be defined in general terms as a rigid rocker model of the foot/ankle complex. A three dimensional rollover shape may be called a rollover surface, and a two dimensional shape may be called a rollover profile.

Studies have shown that a quasi-static rollover profile obtained via bench testing may be highly comparable to a dynamic rollover profile from actual walking.

Studies of various prosthetic feet with the rollover profile methodology have shown that the “effective foot length” during walking is surprisingly short in many cases. For example, in FIG. 58, the size 28 cm SACH foot displayed a functional length of less than 20 cm. The length of the rollover profile is significant for many gait parameters, and recent studies show that it may be relevant to how much oxygen is consumed during gait.

Considering the rollover profile length, along with the recent research into oxygen consumption dynamics, points toward a discrepancy that may be more significant than previously thought. In fact, the energy used in walking may be proportional to the fourth power of the step length. Since the stride length may be equal to the functional foot length plus the distance covered by swing phase, feet with shorter rollover profiles may deliver shorter stride lengths. The average step length is about 0.75 meters, and the difference in rollover profile between a SACH foot and a flex-foot is about 6 centimeters. Considering the relationship described above, one would anticipate a large energy savings by using the longer flex-foot, because the step length is almost 10% greater for the ESF versus over the CF. Surprisingly, this energy savings is not seen in any ESF models with longer rollover profiles. In fact, research shows a small energy savings, on the order of 3%, and some of the research subjects in that study found that some ESF feet were more tiring to use than some CF feet. This correlates well with the experience of clinical prosthetists, who describe that their patients often work against their ESF feet, because their return of power is not biomechanically accurate. Indeed, studies of prostheses show that a very small component of this energy return is in the antero-posterior direction, unlike the natural human limb.

### SUMMARY OF THE INVENTION

An embodiment of the invention includes at least a portion of an artificial prosthetic or orthotic foot for a human, or a robotic foot. The foot may include an artificial midfoot joint, an artificial metatarsophalangeal (MTP) joint, and a mechanical coupler configured to coordinate movement of the artificial midfoot joint relative to the artificial MTP joint.

Various embodiments of the invention may include one or more of the following aspects: the mechanical coupler may be configured to move the artificial midfoot joint into plantarflexion when the artificial MTP joint moves into dorsiflexion; an artificial toe and an artificial forefoot; the artificial MTP joint may connect the artificial toe to the artificial forefoot; an artificial forefoot and an artificial heel; the artificial midfoot joint may connect the artificial forefoot to the artificial heel; the coordinated movement of the artificial midfoot joint relative to the artificial MTP joint may substantially correspond to a coordinated movement of a natural midfoot joint relative to a natural MTP joint during ambulation of a natural human foot; an artificial toe; when a weight is removed from the artificial toe by a contralateral leg heelstrike gait event, the mechanical coupler may be configured provide a spring-like action that pulls the artificial midfoot joint and the artificial MTP joint and cause them to move substantially synchronously, rotating in opposite directions; the spring-like action may release energy that propels the at least a portion of the artificial forward and into a swing phase gait event; an artificial toe and an artificial heel; the mechanical coupler may be configured to coordinate movement of the artificial toe relative to the artificial heel; the mechanical coupler may be a tension member; the tension member may be configured to move the artificial toe substantially synchronously with the artificial heel when the tension member is in a taut configuration; the at least a portion of an artificial foot may be a whole artificial foot; at least one of the artificial midfoot joint and the artificial MTP joint may include a tensegrity joint; the artificial midfoot joint may have a range of motion, from a substantially maximal excursion in a plantarflexion direction to a substantially maximal excursion in a dorsiflexion direction, between about 0.1 degrees and about 120 degrees; the artificial midfoot joint may have a range of motion, from the substantially maximal excursion in the plantarflexion direction to the substantially maximal excursion in the dorsiflexion direction, between about 0.5 degrees and about 60 degrees; the artificial midfoot joint may have a range of motion, from the substantially maximal excursion in the plantarflexion direction to the substantially maximal excursion in the dorsiflexion direction, between about 1 degree and about 30 degrees; the artificial midfoot joint may have a range of motion, from the substantially maximal excursion in the plantarflexion direction to the substantially maximal excursion in the dorsiflexion direction, between about 1 degree and about 10 degrees; the artificial midfoot joint may have a range of motion, from a substantially maximal excursion in a plantarflexion direction to a substantially maximal excursion in a dorsiflexion direction, substantially similar to a corresponding natural human subtalar joint; the artificial midfoot joint may have a range of motion, from a substantially maximal excursion in a plantarflexion direction to a substantially maximal excursion in a dorsiflexion direction, substantially similar to a sum of ranges of motion of a human ankle and human subtalar joints; the artificial MTP joint may have a range of motion, from a substantially maximal excursion in a plantarflexion direction to a substantially maximal excursion in a dorsiflexion direction, between about 0.1 degrees and about 340 degrees; the artificial MTP joint may have a range of motion, from the substantially maximal excursion in the plantarflexion direction to the substantially maximal excursion in the dorsiflexion direction, between about 0.5 degrees and about 60 degrees; the artificial MTP joint may have a range of motion, from the substantially maximal excursion in the plantarflexion direction to the substantially maximal excursion in the dorsiflexion direction, between about 1 degree and about 30 degrees; the artificial MTP joint may have a range of motion, from the substantially maximal excursion in the plantarflexion direction to the substantially maximal excursion in the dorsiflexion direction, between about 1 degree and about 15 degrees; the artificial MTP joint may have a range of motion, from a substantially maximal excursion in a plantarflexion direction to a substantially maximal excursion in a dorsiflexion direction, substantially similar to a corresponding natural human MTP joint; the mechanical coupler may have elastic properties; the mechanical coupler may be configured to store energy; the mechanical coupler may be configured to release stored energy when the artificial midfoot joint and the artificial MTP joint move substantially synchronously, rotating in opposite directions; the mechanical coupler may be configured to compliantly couple the movement of the artificial MTP joint relative to the artificial midfoot joint; the mechanical coupler may be configured such that after the mechanical coupler is pulled taut, the mechanical coupler is configured to allow for an input of energy to one or more portions of the artificial foot as the artificial MTP joint and the artificial midfoot joint and cause them to move substantially synchronously, rotating in opposite directions; the artificial MTP joint may have a range of motion, from the substantially maximal excursion in the plantarflexion direction to the substantially maximal excursion in the dorsiflexion direction, between about 1 degree and about 10 degrees; and the artificial MTP joint may have a range of motion, from the substantially maximal excursion in the plantarflexion direction to the substantially maximal excursion in the dorsiflexion direction, between about 1 degree and about 5 degrees.

Additional objects and advantages of the invention may be set forth in part in the description which follows, and in part may be obvious from the description, or may be learned by practice of the invention. The objects and advantages of the invention may be realized and attained by means of the elements and combinations particularly pointed out in the appended claims.

It is to be understood that both the foregoing general description and the following detailed description may be exemplary and explanatory only and may not be restrictive of the invention, as claimed.

The accompanying drawings, which may be incorporated in and constitute a part of this specification, illustrate several embodiments of the invention and together with the description, serve to explain the principles of the invention.

### BRIEF DESCRIPTION OF THE DRAWINGS

FIG. 1 is a perspective view of a prosthetic foot having a tensegrity MTP joint and midfoot joint, with one big toe, two middle toes, and one little toe, according to an embodiment of the invention;

FIG. 2 is a perspective view of a heel of the prosthetic foot illustrated in FIG. 1;

FIG. 3 is a perspective view of a big toe of the prosthetic foot illustrated in FIG. 1;

FIG. 4 is a side view of a big toe of the prosthetic foot illustrated in FIG. 1;

FIG. 5 is a front view of a toe of the prosthetic foot illustrated in FIG. 1;

FIG. 6 is a perspective view of a forefoot of the prosthetic foot illustrated in FIG. 1;

FIG. 7 is a top view of the forefoot the prosthetic foot illustrated in FIG. 1;

FIG. 8 is a side view of the forefoot the prosthetic foot illustrated in FIG. 1;

FIG. 9 is a front view of the forefoot of the prosthetic foot illustrated in FIG. 1.

FIG. 10 is a bottom view of the prosthetic foot illustrated in FIG. 1;

FIG. 11 is a perspective view of a prosthetic boot according to another embodiment of the invention;

FIG. 12 is a side view of the forefoot of the prosthetic boot illustrated in FIG. 11;

FIG. 13 is a front view of the forefoot of the prosthetic boot illustrated in FIG. 11;

FIGS. 14, 14A, 15A, and 15B are a perspective view of a twist rope midfoot joint, a detail side view, and a rear perspective view of a foot having a twist rope midfoot joint according to a further embodiment of the invention;

FIGS. 16A and 16B are a view from above and a perspective view, respectively, of a prosthetic or orthotic heel having an ankle connecting means according to yet another embodiment of the invention;

FIGS. 17, 18A, and 18B are a side view, rear perspective view, and a rear view, respectively, of a prosthetic or orthotic foot, ankle, and distal leg portion according to a yet further embodiment of the invention;

FIGS. 19A, 19B, and 19C are a rear view, side view, and perspective view of a prosthetic or orthotic lower (or distal) leg having unshaped connecting ends according to still another embodiment of the invention;

FIGS. 20, 21A, and 21B are a front view, perspective view, and (currently upside down) view, respectively, of a prosthetic knee joint and the connections to upper (proximal) and lower (distal) legs portions according a still further embodiment of the invention;

FIGS. 22A, 22B, 22C, and 22D are perspective, front, proximal, and side views, respectively, of an x-brace of a prosthetic knee joint according another embodiment of the invention;

FIGS. 23A, 23B, 23C, and 23D are a distal side view, perspective view, longitudinal end view, longitudinal cross-section of a prosthetic thigh (proximal leg) portion according to a further embodiment of the invention;

FIG. 24 is a perspective view of a prosthetic or orthotic leg, including a foot, ankle, lower leg, knee, and portion of a thigh according to yet another embodiment of the invention;

FIGS. 25A and 25B are an above view and a rear view, respectively, of the prosthetic or orthotic leg illustrated in FIG. 24;

FIG. 26 is a side view of the prosthetic or orthotic leg illustrated in FIG. 24;

FIGS. 27A, 27B, and 27C may be a perspective view, bottom view, and front view, respectively, of a prosthetic heel with a twist configuration midfoot joint according to still another embodiment of the invention;

FIG. 28 is a perspective view of a heel according to a still further embodiment of the invention, for example, for use in a twist midfoot joint;

FIGS. 29A, 29B, and 29C are a perspective view, rear view, and an isometric view of one or more of a great toe, a toe race, a bearing, and a rear toe according to a further embodiment of the invention;

FIG. 30 is a perspective view of a prosthetic foot having one toe connected to a forefoot by a tensegrity MTP joint, and a tensegrity limited twist midfoot joint connecting the forefoot and a heel according to a further embodiment of the invention;

FIG. 31 is bottom view of the prosthetic foot of FIG. 30;

FIG. 32 is a top view of a midfoot twist rope, with the midfoot twist rope complex not shown, according to yet another embodiment of the invention;

FIG. 33 is a front view of the midfoot twist rope, with the midfoot twist rope complex not shown, according to a yet further embodiment of the invention;

FIGS. 34B is a front sectional view of the midfoot twist rope and a midfoot twist rope complex, cut along line A-A of FIG. 34A, according to still another embodiment of the invention;

FIG. 35 is a top view of a coordination rope, illustrating the rope shape, according to a still further embodiment of the invention;

FIG. 36 is a side view of the prosthetic foot from FIG. 30, with arrows indicating the principal directions of motion of the heel and the forefoot during walking gait;

FIG. 37 is a side view of the prosthetic foot from FIG. 30, showing the attachment hardware that connects the ropes to the compression members, or to themselves;

FIG. 38 is a perspective view of the forefoot in FIG. 30;

FIG. 39 is a front view of the forefoot in FIG. 30;

FIG. 40 is a bottom view of the forefoot in FIG. 30;

FIG. 41 is a rear view of the forefoot in FIG. 30;

FIG. 42 is a side view of the forefoot in FIG. 30;

FIG. 43 is a perspective view of the heel in FIG. 30;

FIG. 44 is a side view of the heel in FIG. 30;

FIG. 45 is a front view of the heel in FIG. 30;

FIG. 46 is a bottom view of the heel in FIG. 30;

FIG. 47 is a top view of the heel in FIG. 30;

FIG. 48 is a perspective view of the toe and a race assembly, bearings may not be shown, according to another embodiment of the invention;

FIG. 49B is a front sectional view of the toe, the bearing, and the race assembly of FIG. 49A;

FIG. 50 is a front view of the toe in FIG. 30;

FIG. 51 is a perspective view of the toe in FIG. 30;

FIG. 52 is a side view of the toe in FIG. 30;

FIG. 53 is a perspective view of a race, showing hidden lines as dashed lines, according to a further embodiment of the invention;

FIG. 54 is a front view of the race of FIG. 53;

FIG. 55 is a side view of the race of FIG. 53;

FIG. 56 depicts the Windlass Mechanism, where with dorsiflexion of the MTP, the plantar aponeurosis stores energy by vaulting the longitudinal arch;

FIG. 57 depicts Instrumented Gait Analysis of the foot where TB=Tibia, FL=Floor, HF=Hindfoot, FF=Forefoot, and HX=Hallux (great toe);

FIG. 58 depicts rollover profiles showing how different types of feet flex under loading;

FIG. 59 is a perspective view of a heel with midfoot outer sheaves that may be used with a twist midfoot rope, according to still another embodiment of the invention;

FIG. 60 is a perspective view of a basic tensegrity universal joint according to a still further embodiment of the invention; and

FIGS. 61A-61B are a top and side view of a foot including a limit twist midfoot joint according to yet another embodiment of the invention.

### DESCRIPTION OF THE EMBODIMENTS

Reference may now be made in detail to various embodiments of the invention, examples of which may be illustrated in the accompanying drawings and set forth in the specification. Wherever possible, the same reference numbers may be used throughout the drawings to refer to the same or like parts.

Embodiments of the device proposed in this application may be based on the tensegrity design idea. Tensile-integrity, shortened to “tensegrity,” may refer to a special type of structure comprising continuous tensile members (e.g., cables) acting upon discontinuous compressive members (e.g., spars). Tensegrity structures may rely upon the tensile strength and flexibility properties of wire rope to bear physical loads placed upon them. Major innovations in steel wire rope technology, driven by increasing performance demands in the automotive and aerospace sectors, now permit the construction of light weight joints that may be stronger in many cases than traditional engineered “beam and bearing” structures.

As used herein, “tensegrity” may refer to the characteristic of having two or more discontinuous compression members dispersed in a network of one or more continuous tension members.

As used herein, “tensegrity joint” may refer to a joint having a tensegrity structure. In a tensegrity joint, the two or more discontinuous compression members may be incompletely constrained by the network of the one or more continuous tension members in which they may be dispersed, whereby the compression members may be able to move relative to each other. The movement may be at one or more centers of motion, and optionally around a primary axis at each center. Optionally the primary axis may be virtual and not coaxial with an actual cable.

As referred herein, dorsiflexion may be defined as motion in the direction of the top of the foot (e.g., dorsal surface), plantarflexion may be defined as motion in the direction of the bottom of the foot (e.g., plantar surface), and excursion may be defined as a movement from and back to a mean position or axis in an oscillating or alternating motion and/or the distance traversed in such a movement.

As referred to herein, “stop compliantly” may be defined in the following context: a rigid stop may be modeled as a step function, where in one instant before the step, one may be able to move with no restriction or required increase of force and afterward no motion is possible. Thus, if a rigid stop is a step function, then a compliant stop would be a sloped line, rather than a step function. Although some materials may in theory exhibit properties similar to a rigid stop, in practice, even prestretched steel wire rope may not be a step function and may have at least some slope, but still may not “stop compliantly”. As described herein, however, an elastic limit rope may be more sloped than then prestretched steel wire rope, and hence may “stop compliantly.”

As referred to herein, superior may be defined as roughly equivalent to up, or in the direction of the head. Inferior may be defined as roughly equivalent to down, or in the direction of the bottom of the feet. These terms may refer to a body in “standard anatomic position,” which is standing straight up, palms facing forward, feet facing forward.

As referred to herein, a lateral side may be a side away from a midline of the body, and a medial side may be a side toward the midline of the body. For example, a toe may have a lateral side and a medial side where a surface of the toe facing “outward” may be a lateral side, a surface of the toe facing “inward” may be a medial side, when the toe is connected to a person. If the toe (or any other part) is considered alone, without a larger context, then medial and lateral may refer to the midline of that part, as divided along a midsaggital plane.

As set forth herein, a Torque/Angular Deflection Response Curve may be defined as follows: just as a cantilevered beam may bend further with an increasing load on it' free end, a rotational, angular deflection response may be characterized for a joint with a moment applied on it. This moment (torque) may be a product of a force applied to one of the compression members that makes up the joint. For example, if a force is applied to a toe, the MTP joint may experience a torque. This torque may move the joint, until the restoring force of the joint is equal to the torque. If the toe has a “spring” or an actuator attached to it, or some other way of influencing the torque response, then the torque/angular deflection curve may be different than it was without this spring or actuator. Natural human joints may have a torque/response curve that is predominantly a function of the positions of muscular attachments relative to the centers of motion of the joints, and the strengths of those muscles. The neurological control of these muscles may also be relevant. A prosthetic, orthotic, or robotic version of a human joint may use stronger materials than the tendon, muscle, and bone of human joints, thus may achieve similar, or very different, torque/angular deflection response curves. Computer controls may be employed to effect a changing torque/angular deflection response curve.

As set forth herein, an ultimate compressive strength of a structure may be how much loading the structure can withstand before it may experience a mechanical failure. Mechanical failures of human limbs may include sprains, strains, and tears of ligaments and tendons, and/or fractures of bones.

In a tensegrity joint, the compression members may be rigid, and the number, length, diameter, geometric organization, and flexibility characteristics of the tension members may determine the range of motion of the compression members. Tension members may constrain and/or stabilize the compression members.

The movement or motion of a first joint may be coupled to the movement or motion of a second joint using a coupling cable. The coupling cable may directly connect two compression members that may not be directly connected by a single joint, but indirectly connected by two or more joints.

Each joint in the animal body may have its own specific geometry. Joint(s) in embodiments of the invention may be designed to have similar characteristics of natural joints. Alternatively, super joints may be designed for prosthetics, orthotics, and robotics that do not interfere with the functioning of the remaining joints of the body or robot.

The compression and tension member materials may be selected to maintain structural integrity considering the use of the device and the user of the device. Devices that must withstand greater forces may be made from stronger components.

As used herein, “rope” may refer to an element capable of functioning as a tension member in a tensegrity joint including cables having a diameter less than about ¼ inch. An effective rope element may comprise two or more thinner ropes, including a thin rope making multiple passes and having a larger effective diameter than the thin rope alone. Generally, these ropes may not be elastic. Elastic elements of the joints of the invention may be termed actuators. Actuators used in embodiments of the invention may be elastic and may have a spring-like quality. Alternately, liquid-metal, a new material innovation, may be used for the ropes and/or for compression members. Liquid-metal may have some elastic properties.

As used herein, “polycentric joint” may refer to a joint having two or more centers of motion that move concurrently. For example, the knee may be a polycentric joint, having two centers of motion. Each center of motion may be defined by a universal joint. Two universal joints may be stacked on top of each other. One compression member may be directly connected to tension members in each joint. The directly connected compression member may have two U-shaped ends in the same plane as each other and facing opposite directions. This part may be called the x-brace.

Orthotics may augment body parts. Prosthetics may replace body parts. Robotics may function similarly to body parts but may not require direct connection to a body in order to be functional.

Limit ropes and stabilization ropes may be tension members in tensegrity joints that limit the ranges of motion of the joint. Optionally the tensegrity joint may have a primary axis of motion. A primary axis of motion may be the least constrained axis of all the other axes of motion of the joint. Optionally one tension member may be coaxial with the primary axis of motion of the joint. Alternatively, the primary axis of motion may not be coaxial with a tension member, and the primary axis may then be said to be virtual.

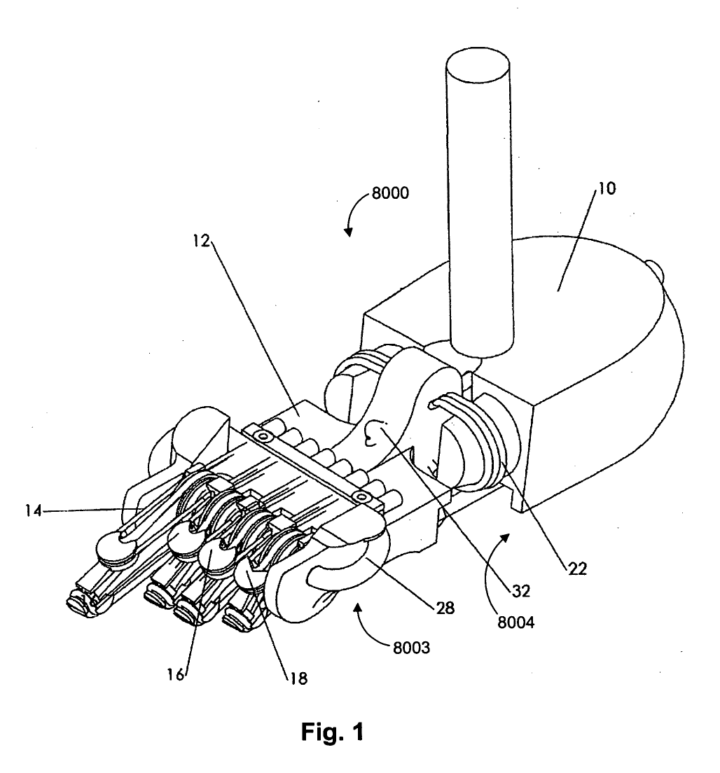

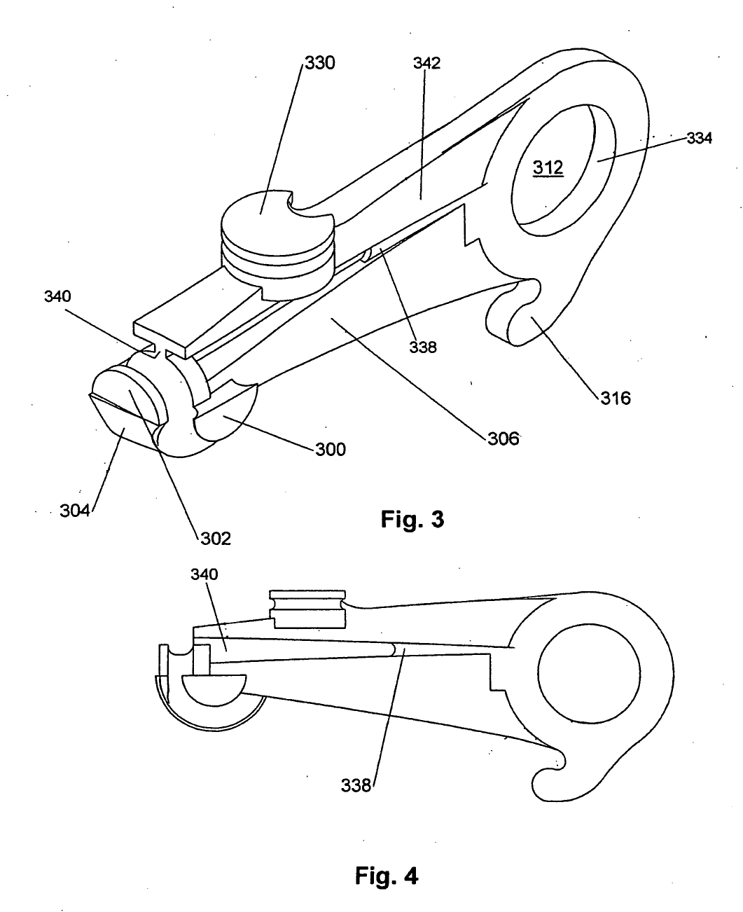

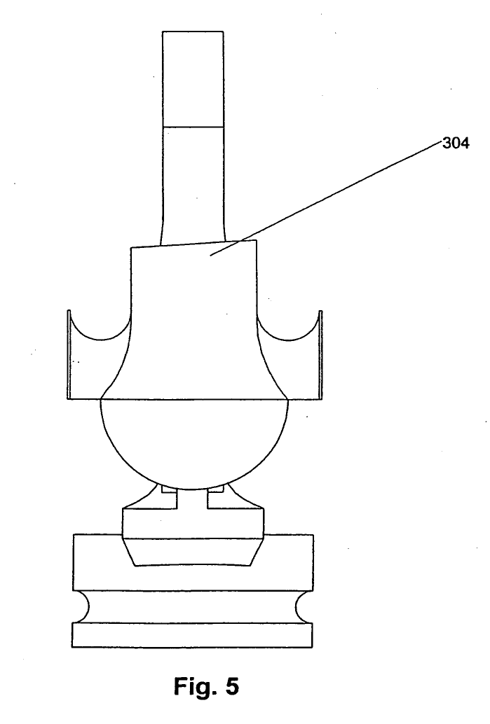

The parts and configuration of the toes 14/16/18 may be seen in FIGS. 1, 3-5, and 10. The inner surfaces of the top sheave 302 and the side sheaves 300 (only one shown) may form a continuous path. This may allow the extension limit rope 38 to wrap around these structures without damage to the rope.

The angled walking surface 304 may contact the ground (or the inner surface of the cosmesis if one is used). The angle may be dictated by the angle between the midfoot attachment of the forefoot 12 and the toe supports 102 of the forefoot 12. In general, the toe 14/16/18 may sit at a slight (about 4 degree) angle to the ground, with the angled walking surface 304 in contact with the ground. This surface may be rounded, so that the toe 14/16/18 may be stable during the walking stride, for example, rolling over without wobbling.

The main column 306 of the toe may be the primary weight-bearing column. Most of the loading that the toe sustains may be columnar loading of the long axis, as the extension limit rope 38 may press against the side toe sheaves 300, which may push the toe into the axial rope 28.

The axial rope hole 312 may be the void in the toe that may allow the axial rope 28 to pass through the toe. This hole 312 and the rope 28 may act as the center or primary axis of rotation for the toe motion. The axial rope hole 312 may be considerably larger than the axial rope 28 used, because a smaller hole may act as a ‘stress concentrator’ and cause premature failure of the toe, under maximum loading conditions. The oversized hole 312 also may allow for bearings 383 and a race 380 to be placed between the toe and the axial rope 28, protecting the toe from being damaged by the rope, and vice versa. The toe may damage the strands of the rope 28, precipitating a rope failure, or the strands of the rope 28 may carve away at the inner surface of the axial rope hole 312, eventually wearing away the material and weakening the structure. These scenarios of carving or rope breakage may be generalized to any place where ropes contact metal surfaces. Ways to avoid this damage may be by using tough coatings for the metal surface, or flexible coatings for the steel wire rope.

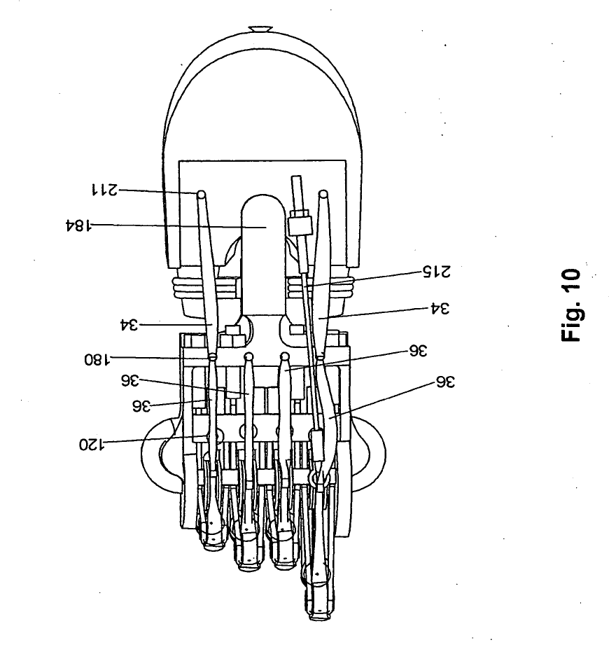

The actuator hook 316 may allow the attachment of actuators 34 (FIG. 10), springs, or additional cables to the toe 14/16/18, allowing for active or passive control of the toe's motion.

The top sheave 330 may be where the flexion limit rope 40 wraps the toe. When the rope 40 is in a taut configuration, the toe 14/16/18 may no longer move around the axial rope 28 in the “flexion” direction. This may be the direction that the actuator 34 pulls on the actuator hook 316.

The top beam 342 may stiffen the main column 306 of the toe, similar in function to an I-beam. This may reduce the deflection that the toe exhibits under loading conditions.

The thin web 340 may be also similar in function to the web of an I-beam. The thin web 340 may join the top beam 342 to the main column 306, and may be thin enough to release the shear stress experienced under loading, without failing.

The thick web 338 may be located at a critical point in the thin web 340. Shear stress may concentrate at this point and may cause failure of the thin web 340, so the web may be reinforced at this point.

The bearing clearance 334 may be a smooth, annular surface around the axial rope hole 312. Bearings 383 (FIG. 1) may be mounted to this point, lubricating the motion of the toe 14/16/18 around the axial rope 28, and protecting the toe from damaging the toe supports 102 of the forefoot 12, and vice versa. This may be also relevant for smooth operation of the device, because the toes may otherwise bind on the toe supports 102, causing poor operation of the device, and possible damage or failure.

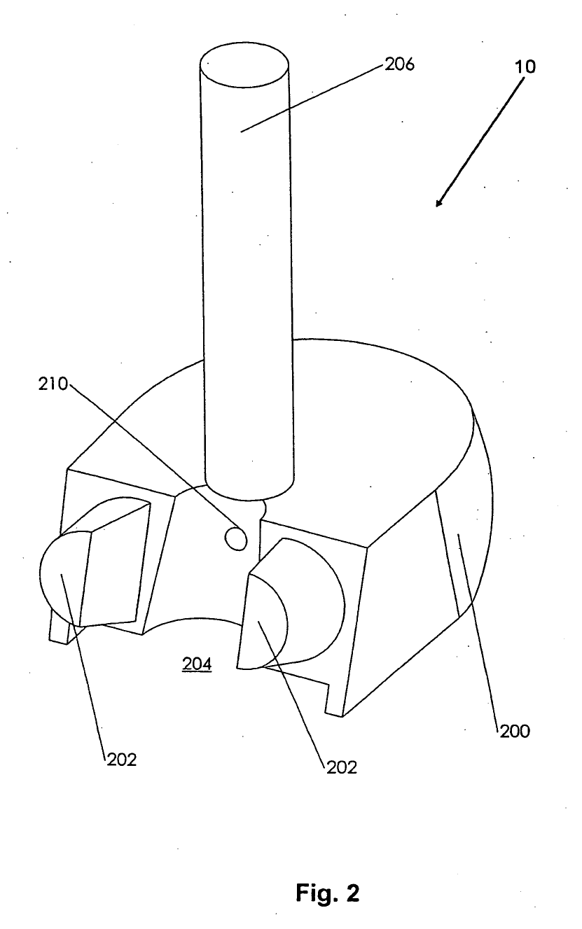

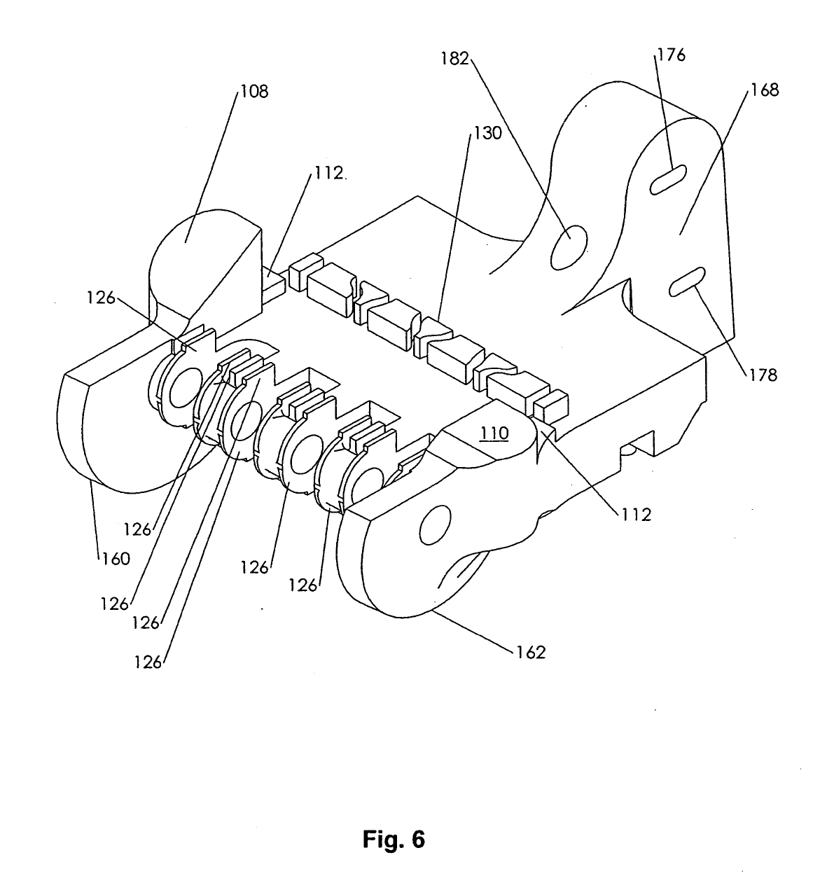

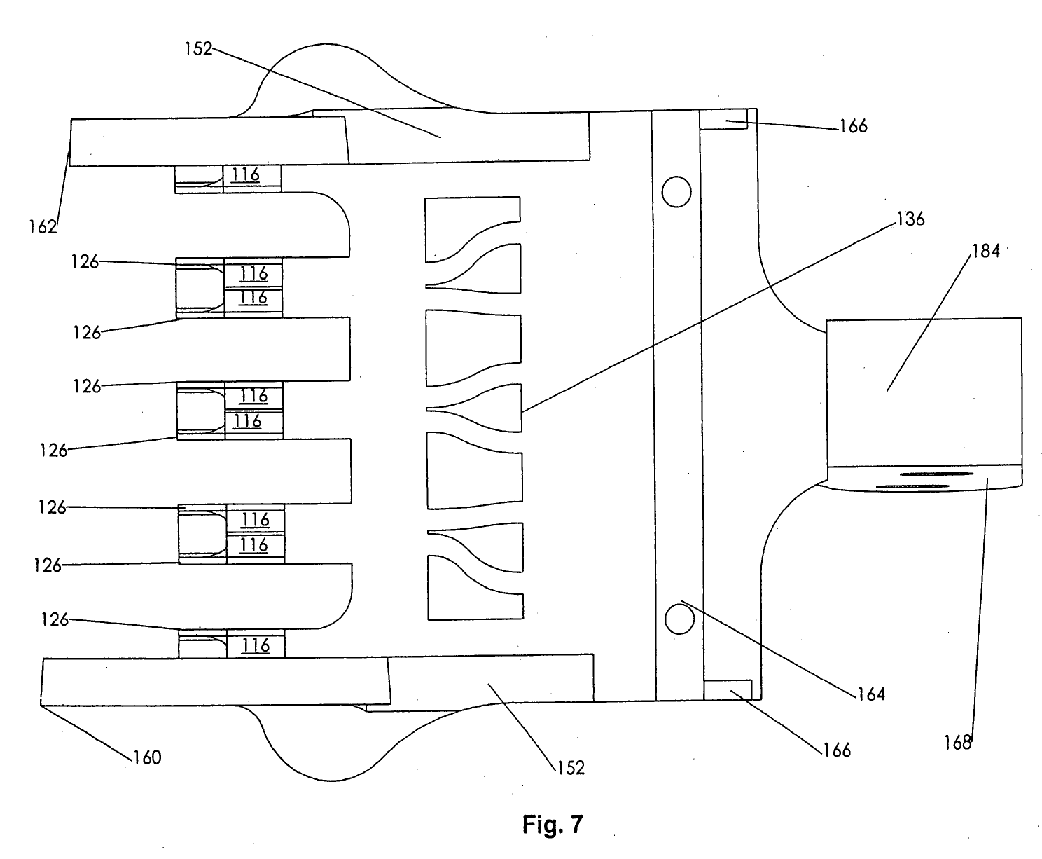

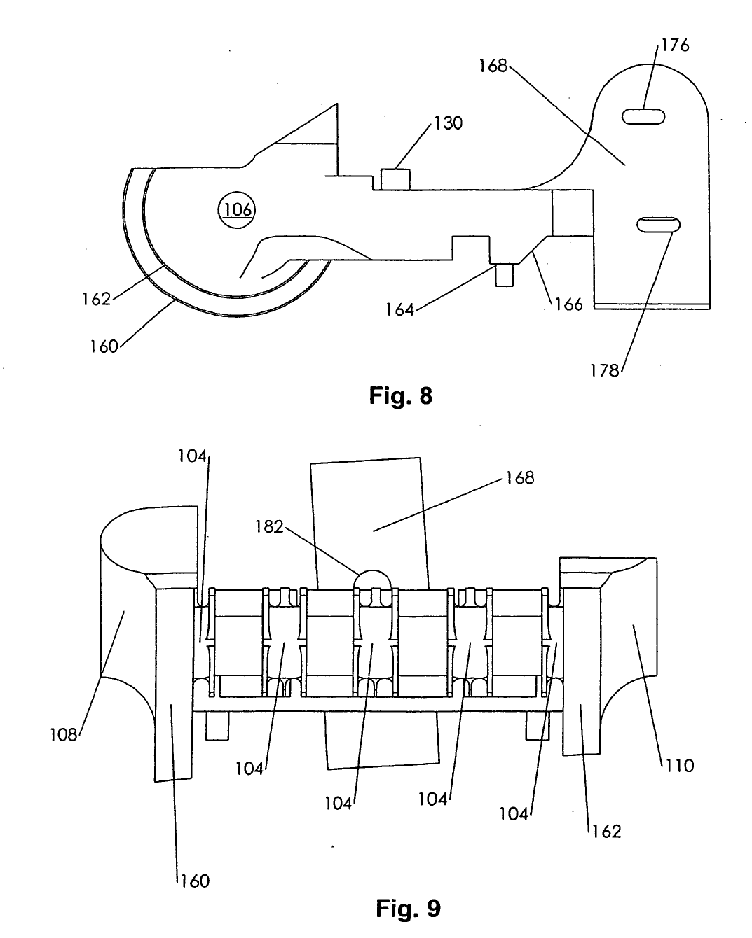

Note that the prosthetic foot illustrated in FIGS. 1-10 may include three different sized toes, for a more natural gait. For example, a big toe 14, two middle toes 16, and a little toe 18, are shown in FIG. 1.

The parts and configuration of the heel may be seen in FIGS. 1, 2, and 5. The cushioned heel 200 may protect the amputee against shock loading of their residual limb. The cushion may be made of foam or other padding material and may have preload adjustments or other ways of adjusting the hardness of the cushion.

The midfoot joint outer sheaves 202 may be used to connect the heel 10 to the forefoot 12, forming relevant components of the midfoot joint's rear compression member. Wire rope 22, or other tensile members may wrap these sheaves 202. The midfoot joint outer sheaves 202 may have a conical draft. This draft may be relevant in the formation of the midfoot joint because their increasing diameter may prevent the heel 10 from moving too close to the forefoot 12. Also, without this draft, the midfoot tightener rope 32 may not function as needed to stabilize the joint.

The movement clearance 204 may be a void, providing clearance for the movement of the forefoot 12 relative to the heel 10, without direct contact between these two structures. Though the movement clearance 204 may be shown as semi-circular, this need not be the case.

A pylon 206 optionally may attach to the amputee's socket, for example, the interface between their stump and the prosthesis. Alternatively the pylon 206 may attach to another component, such as a knee. The pylon 206 may be any length, and it need not be an integral part of the heel 10. It may be an after-market part that mates to the heel 10. It may be fitted with any manner of attachment mechanism known in the art of prosthetic devices.

The tightener rope hole 210 may pass through the heel 10, which may allow the midfoot tightening rope 32 (FIG. 1) to span the midfoot joint.

The heel actuator stays 211 may be placed where the midfoot joint actuators 34 may attach to the heel 10. They may sit in a void in the model, positioned so that they may not be weight bearing in function during the walking stride. Cotter pins or other mechanisms may be used to ensure that the midfoot actuators 34 do not slip off these actuator stays 211.

The parts and configuration of the forefoot may be seen in FIGS. 1, 3, 4, and 6-9. The toe supports 102 may be the protrusions from the front of the forefoot 12. They may hold the axial ropes 28 in a stable position while the toes may be under load. They also may provide a place for several other features.

The side toe sheaves 300, distal toe sheave 302, and top sheave 330 may form rounded surfaces that support the dorsiflexion limit ropes 38 and the plantarflexion limit ropes 40 when they may be under load. Specifically, the side toe sheaves 300, top sheave 302 may support the extension limit rope 38. The sheaves may protect the ropes from kinking by guiding their bending.

The limit rope guides 126 may be located on the edges of the front sheaves 104, and may further guide the plantarflexion limit ropes 40 and the dorsiflexion limit ropes 38 by preventing these ropes from slipping off of the sides of the front sheaves 104. The axial rope hole 312 may pass through the toe supports 102, providing a place for the axial rope 28 to pass.

The tall axial sheave 108 and the short axial sheave 110 may provide support for the bends in the axial rope 28, protecting the rope 28 in a similar manner to the other sheaves, supporting it so that it may not kink. The short axial sheave 110 may be shorter than the tall sheave 108 because of the need to mimic the shape of the human foot. The ball of the great toe in a normal human foot may be significantly larger than the ball of the little toe. Since this device may fit into a shoe, it may mimic this shape.

The spacer 112 for the plantarflexion ropes 40 may ensure that the axial rope 28 may not slip down to the base of the tall axial sheave 108 and the short axial sheave 110. If this happened it may block the path of the plantarflexion limit ropes 40. Damage to the axial rope 28, or to the plantarflexion limit ropes 40 may occur in this situation.

The dorsiflexion limit rope holes 116 may be “open” holes, for example, holes that have no top wall. This may ease the assembly of the dorsiflexion limit ropes 38 and the MTP joint in general. These dorsiflexion limit holes 116 may have a rounded bottom, which may properly support the extension limit rope 38.

The plantarflexion limit rope holes 120 may be “open” holes, for example, holes that have no bottom wall. This may ease the assembly of the plantarflexion limit ropes 40 and the MTP joint in general. These plantarflexion limit holes 120 may have a rounded top, which may properly support the extension limit rope 38.

The plantarflexion limit rope spreader 130 (which may be the same part as the dorsiflexion rope spreader 136) may change the spacing of the plantarflexion limit ropes 40. The spacing of the ropes 40, as they pass through the plantarflexion limit rope holes 120, may be dictated by their function within the MTP joint design. The spacing of these holes may not be sufficient to allow for parts to fit in and connect these ropes 40 to the forefoot 12, so that they may be rigidly attached to the device. Thus, the plantarflexion limit rope spreader 130 may change this spacing, spreading the ends of the plantarflexion limit ropes 40 apart. The plantarflexion limit ropes 40 may be specified with a safety factor of at least ten, making the use of stop swages 44 permissible, by the art of wire rope design. The plantarflexion limit rope spreader 130 may also serve to stop the motion of these stop swages 44, for example, by blocking their motion. In the discussion of the function of the MTP joint, there may be further discussion of this function.

The dorsiflexion limit rope spreader 136 may change the spacing of the dorsiflexion limit ropes 38. The spacing of the ropes 38, as they pass through the dorsiflexion limit rope holes 116, may be dictated by their function within the MTP joint design. This spacing may not be sufficient to allow for connecting these ropes 38 to the forefoot 12, so that they may be rigidly attached to the device. Thus, the dorsiflexion limit rope spreader 136 may change this spacing, spreading the ends of the dorsiflexion limit ropes 38 apart. One of these ends may then be attached to the screw attachment bar 164 by means of a screw swage 42. The motion of the other end may be blocked by the interference of the stop swage 44 and the dorsiflexion limit rope spreader 136. These screw end swages 42 may pass through the screw attachment bar 164, and may be bolted in place. The screw end swages also may allow for some adjustability of the range of motion of the MTP joints after assembly, as the screw ended swages 42 may be bolted and locked in a variety of lengths.

The screw attachment bar 164 may have holes in it that may be spaced, according to the rope spacing, as the dorsiflexion limit ropes 38 after they emerge from the dorsiflexion limit rope spreader 136. This screw attachment bar 164 may provide a strong support to which the extension limit rope 38 may be attached.

The forefoot actuator stays 180 may extend downward from the screw attachment bar 164 and may serve as a point of attachment for the actuators/springs 34 for the midfoot joint and/or the MTP joints.

The medial angled walking surface 160 and the lateral angled walking surface 162 and the rear weight platform 184 on the heel 10 may create a tripod for stable weight bearing. These surfaces may all be on the same angle, for example, relative to the rest of the forefoot 12. This may be because of the need to mimic the geometry of the human foot, with its tapered shape, for example, large on the medial side and narrow on the lateral side. The medial angled walking surface 160 may be a good deal larger than the lateral angled walking surface 162, for example, in accord with this geometry. These angled walking surfaces may both be large enough to provide clearance underneath the forefoot 12 so that there may be room for the actuator hook of the toes 316, the toe actuators 36, and the dorsiflexion limit ropes 38. The stiffener bars 152 may add strength and stiffness to the forefoot 12, making the forefoot 12 more able to deal with maximal loading conditions on the medial angled walking surface 160, and the lateral angled walking surface 162.

The midfoot joint attachment may be a very strong structure because it may function in critical weight bearing roles. It also may provide a place for a number of other features. The midfoot joint tightening rope 32 may pass through the tightener rope hole 182. This hole 182 may pass through the midfoot joint attachment 168. The upper midfoot rope hole 176 and the lower midfoot rope hole 178 both may pass through the midfoot joint attachment 168 as well. These holes may be very much like sheaves, for example, in that they may protect the wire ropes from damage by guiding the path of the rope. These holes may be curved, for example, rather than passing straight through. For ease of manufacture, they may be cut as windows, e.g., squared holes with a curved surface.

All sheaves, or sheave-like holes, mentioned herein may (or may not) be protected from damage by using a coating or a cover, some of which may be known in the art. Such a coating or cover may be tough enough to withstand the “sawing” of the wire ropes, for example, such that the underlying structures are not damaged or abused during normal service.

The twist configuration of the midfoot joint may differ from the midfoot joint shown in FIG. 1, primarily because the geometry of the midfoot joint ropes 52 may be very different. The twist rope may wrap around itself, twisting like a common baggie tie, on either side of the midfoot attachment. Also the midfoot outer sheaves 212 may be conical. The dorsiflexion of the midfoot joint may increase the amount of this twist. The reason for this alternate configuration of the midfoot joint may be that the limits of the dorsiflexion may be easier to predict and control. Midfoot attachment hole upper 187, and lower 188, may be similar in function to upper midfoot rope holes 176 and 178 respectively, but they may curve in the opposite direction. This curve may be dictated by the rope path. The creation of the twist in the rope 22 by looping around the round midfoot outer rope sheaves 212 may create a different path for the rope 52, so that the curve of the rope path may be concave, rather than convex.

Also the alternate plantarflexion limit rope sheave 189, sheave structural support 190, plantarflexion limit rope guide 191, plantarflexion sheave surface 192 may be added to constrain the plantarflexion of the midfoot joint by the attachment and addition of the plantarflexion limit rope 54. The twist may be tight, to the point of stretching the steel wire rope 54, so that it may be difficult to force motion in the plantarflexion direction by more that 10-15 degrees. This tension may be preferably maintained, or the twist may try to unwind by bending in the plantarflexion direction. Thus, the plantarflexion limit rope 54 may block this motion.

As in the early description of the midfoot joint, the taper of the outer midfoot sheaves 212 may be relevant. Without a taper, tightening the tightener rope 32 may simply pull the twist rope further down on the outer midfoot sheaves 212. The taper may keep the rope from sliding to the base of the sheave, where it may meet the angled plate 218. Opposing this sliding action may store energy, stabilizing the joint, and constraining the range of motion.

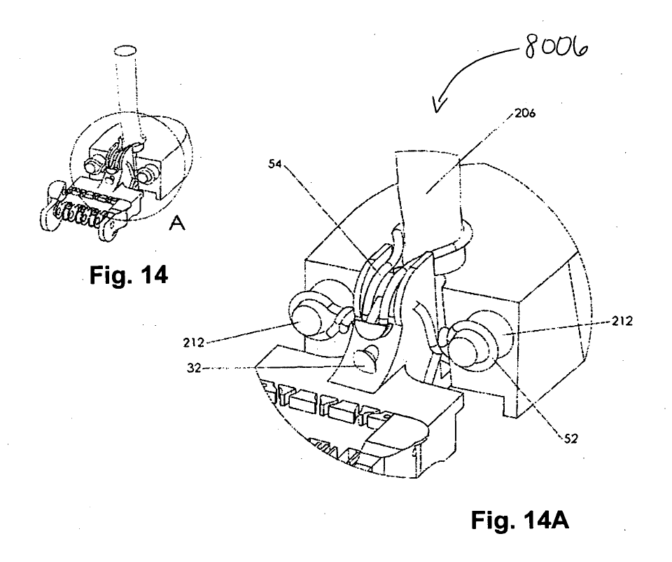

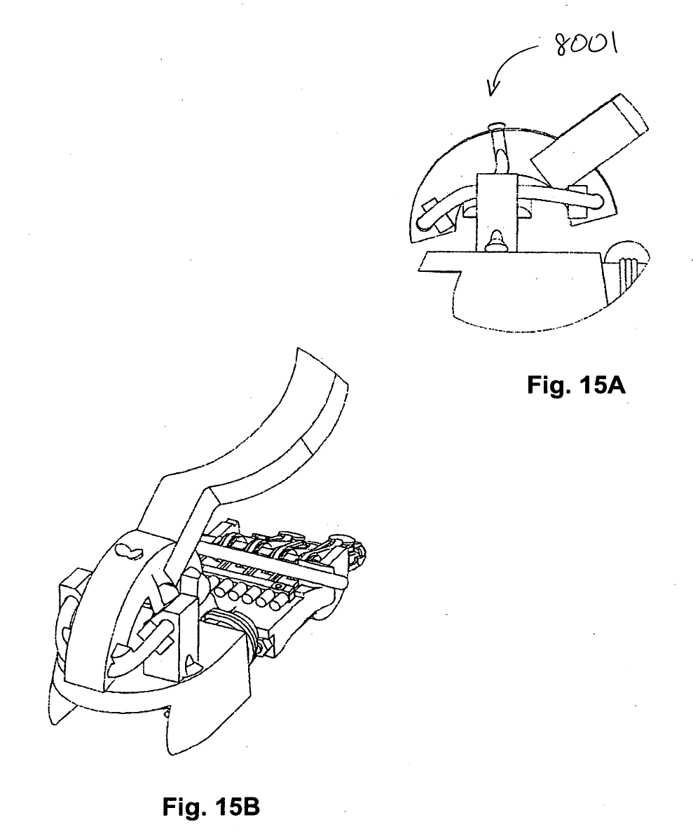

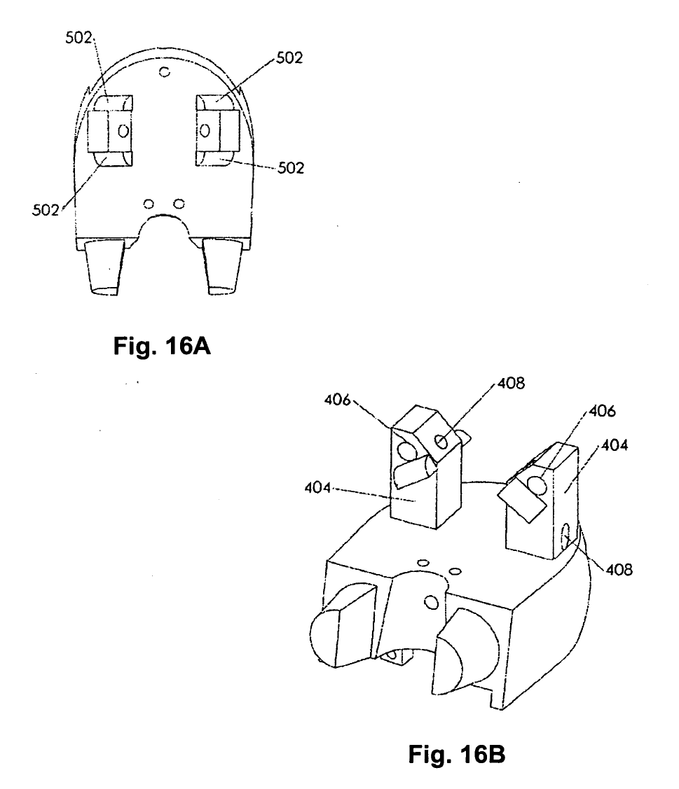

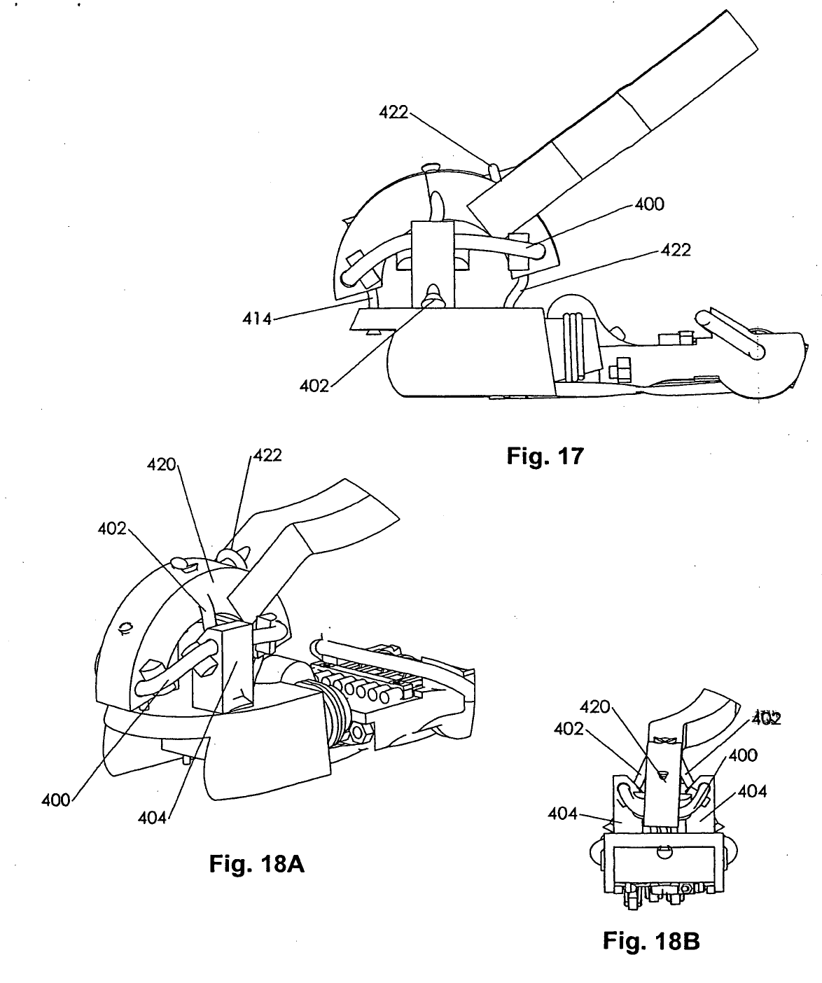

FIGS. 14-18 may show the parts and configuration of the ankle joint. Ankle universal rope 400, a tension member, may be a steel wire rope, joined to itself, or to the adjacent compression members, in accordance with the art of working with steel wire ropes. This rope may keep the lower leg 450 and the heel 10 from collapsing against each other. It also may provide an axis for motion in any direction that may not be constrained. It may pass through the heel universal rope holes 110 and the lower leg universal rope holes 430 and may wrap around the ankle sheaves 502 for protection.

Ankle stabilizer ropes 222 may stabilize the ankle by constraining the motion in the medio-lateral (side to side) direction. They may pass through the lower leg stabilizer rope holes 412 and the heel stabilizer rope holes 408. The ankle stabilizer ropes 402 may be joined to the heel attachment stubs 404 and the u-shaped end of the lower leg 420 in accordance with the art of working with steel wire ropes. These ropes may also function in providing rotational stability.

Heel attachment stubs 404 may be the protuberances rising from the heel 10, for example, for the purpose of attaching the ankle universal rope 400 and the ankle stabilizer rope 402 to the heel 10. The heel universal rope holes 406 and the heel stabilizer rope holes 408 may be located on the heel attachment stubs 404. The heel attachment stub 404 also may have sheaves 502 on it to protect the ankle universal rope 400.

U-shaped end of lower leg 420 may be the site of the lower leg universal rope holes 410 and the lower leg stabilizer rope holes 412. It may exist so that the lower leg 450 may be attached to the ankle universal rope 400 and the ankle stabilizer rope 402. It also may have sheaves 502 on it to protect the ankle universal rope 400.

Sheaves 502 may be rounded compression members that the wire ropes wrap around so that they may not be kinked or otherwise damaged by the motion of the joint. Sheaves 502 may appear on all compression members where the universal joint rope 400 wraps around a compression member.

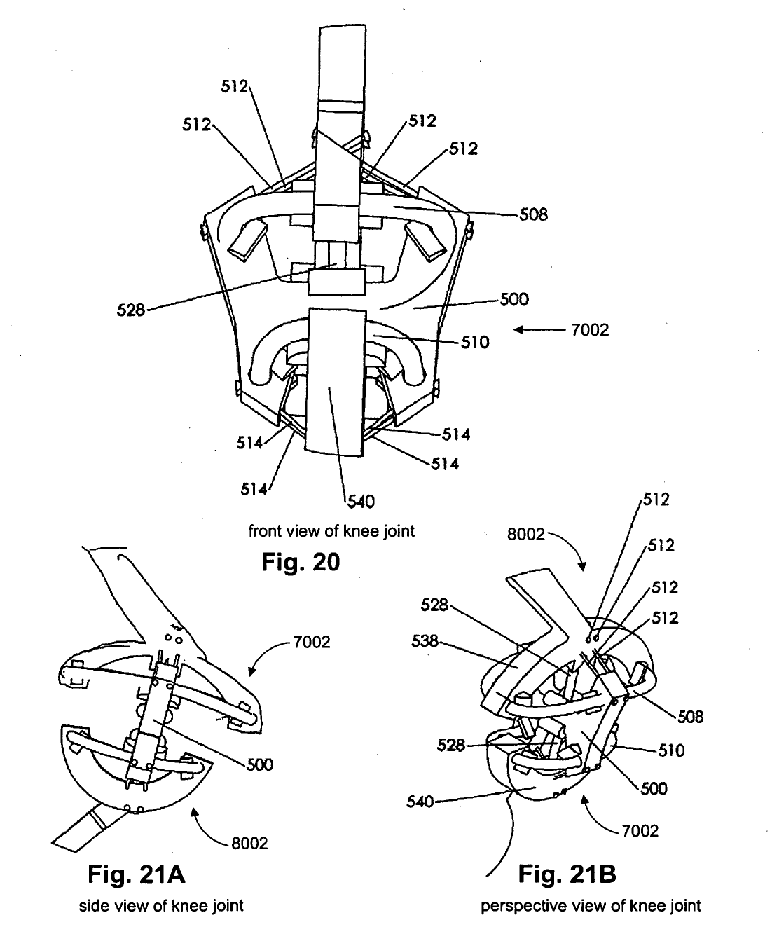

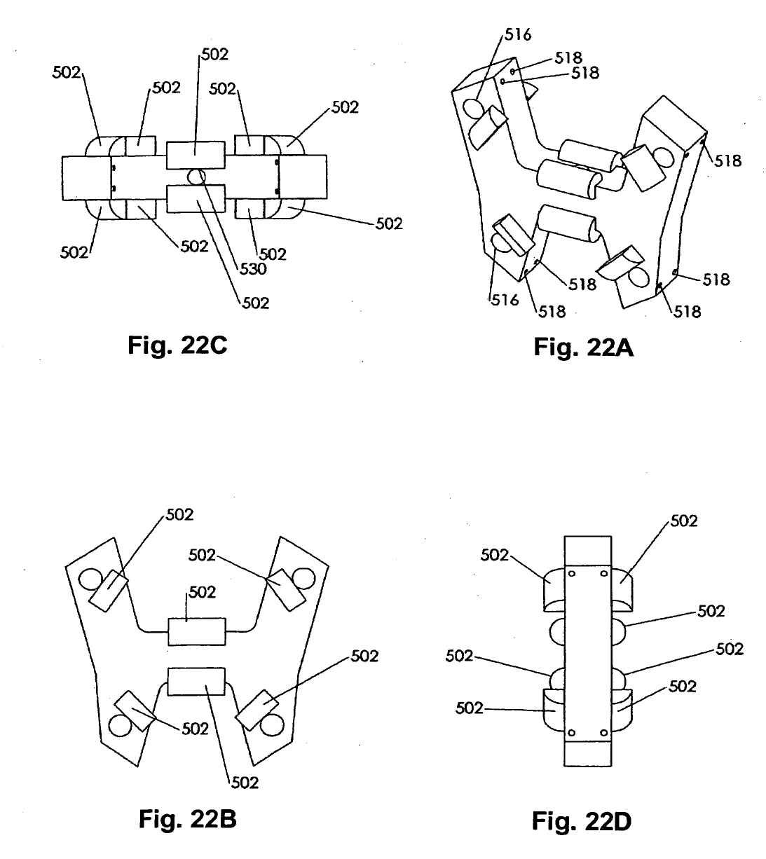

The parts and configuration of the knee may be seen in FIGS. 20, 21A-21B, and 22A-22D. The x-brace 500 may be a compression member that sits between the upper joint member 504, at least a portion of a thigh, and the lower joint member 506, shin or pylon. The x-brace 500 may provide two centers of motion for the knee joint, as may be observed in the natural human anatomy. The x-brace 500 also may have sheaves 504 on it so that the universal ropes 508/510 may not be kinked or otherwise damaged by operation of the joint.

Sheaves 504 may be rounded compression members that the wire ropes wrap around so that they may not be kinked or otherwise damaged by the motion of the joint. Sheaves may appear on all compression members where the universal joint ropes 508/510 wrap around a compression member.

The upper joint member 504, analogous to the human thigh if this joint, may be considered a knee and may be a compression member. The U-shaped end of the upper joint member 538 may be large enough to accommodate the universal joint rope holes 520 and the sheaves 504. The radius of the U-shaped end of the upper joint member 538 may be considerably larger than the U-shaped end of the lower joint member 540 so that when the joint bends, the U-shaped end of the upper member 538 may not collide with the U-shaped end of the lower joint member 540.

The lower joint member 506, may be analogous to the human lower leg, or the prosthetic pylon in artificial limbs. In function it may be very similar to the upper joint member 504. The U-shaped end of the lower joint member 540 may be large enough to accommodate the universal joint rope holes 522 and the sheaves 502, but still small enough to function as described above.

The upper knee universal rope 508, a tension member, may be a steel wire rope, joined to itself, or to the adjacent compression members, in accordance with the art of working with steel wire ropes. This rope 508 may keep the upper joint member 504 and the x-brace 500 from collapsing against each other. It also may provide an axis for motion in any direction that may not be constrained. It may pass through the upper joint member universal rope holes 520 and the x-brace universal rope holes 516 and may wrap around the sheaves 502 for protection.

The lower knee universal rope 510, a tension member, may be a steel wire rope, joined to itself, or to the adjacent compression members, in accordance with the art of working with steel wire ropes. This rope 510 may keep the lower joint member 506 and the x-brace 500 from collapsing against each other. It also may provide an axis for motion in any direction that may not be constrained. It may pass through the lower joint member universal rope holes 522 and the x-brace universal rope holes 516 and wraps around the sheaves 502 for protection.

The upper knee stabilization ropes (cables) 512 may constrain medio-lateral motion (e.g., side-to-side motion) between the upper joint member 504 and the x-brace 500. They may pass through the upper joint member stabilization rope holes 524 and the x-brace stabilization rope holes 518, terminating with rigid or adjustable swage fittings as may be appropriate, according to the art of steel wire rope design.

The lower knee stabilization ropes (cables) 514 may constrain medio-lateral motion (e.g., side-to-side) between the lower joint member 506 and the x-brace 500. They may pass through the lower joint member stabilization rope holes 526 and the x-brace stabilization rope holes 518, terminating with rigid or adjustable swage fittings as appropriate, according to the art of steel wire rope design.

The knee tightener rope 528 may prevent the upper joint member 504 and the lower joint member 506 from moving away from each other. It may pass through the x-brace tightener rope hole 530, the upper joint member tightener rope hole 532, and the lower joint member tightener rope hole 534. It may terminate with rigid or adjustable swage fittings as appropriate, according to the art of steel wire rope design. This rope may be also protected from damage by sheaves 502.

In the ”neutral” or “at rest” configuration, the actuator/spring's pull on the actuator hook of the toe may be blocked by the plantarflexion limit rope wrapping around the forefoot toe support sheaves and the plantarflexion limit rope toe sheave.

As the toe may be loaded by forces on the toe-angled walking surface, it may swing around the axial rope, riding on the bearing surface and the toe race. The axial rope may see little force at this time, as most of the weight may be born by the forefoot - medial and lateral angled walking surface. As the toe swings, force from the actuator may peak and then may decrease, in a function defined by the position of the actuator hook relative to the axial rope, and the force/length curve of the particular actuator/spring. While the toe may be swinging in the dorsiflexion direction, it may be pulling the dorsiflexion limit rope taught. When the toe no longer pulls the dorsiflexion limit rope, because the dorsiflexion limit rope may be at its maximum length, the toe may be at the limit of its range of motion. At this point, the main column of the toe may be heavily loaded, and in turn, may heavily load the axial rope. The toe may then be static, for example, suspended between the force pressing on its angled walking surface, the force of the dorsiflexion limit rope on the distal toe sheave and the side sheaves, and the resultant force of the axial rope. There may be some “twist” of the toe, for example, around its long axis, by virtue of the non-orthogonal force arrangement. Specifically, the angled walking surface of the toe may cause this twist. The twist may be expressed and/or blocked by the toe twisting against its bearings and the toe race. The toe race may press into the forefoot-toe supports, and its motion may be stopped. The toe may still be free to move around the axial rope, as the loading forces, actuator/spring force, and dorsiflexion limit ropes allow, because the bearings may prevent it from binding on the toe supports. Also, irregularities in terrain may cause similar twisting motions of the toes, wherein the bearings may function in much the same way.

At any point in this movement cycle, the limit ropes may come out of their guides, so bottom rope keepers may be used to cover the “exits” from the proscribed rope pathway. The top swage and rope keeper may fit over the plantarflexion rope spreader, making sure that the ropes and stop swages do not come out of the spreader. Rope guide keepers may fit between and over the limit rope guides, assuring that the limit ropes do not leave their track in this location. The bottom rope keeper may work similarly to the top swage keeper, but may attach to the dorsiflexion rope spreader, keeping dorsiflexion limit ropes properly seated.

Starting from the “flat” or “neutral” configuration, there may already be some tension built into the joint. Like at least some of the wire ropes in these designs, the midfoot ropes may not be extensible. For example, they may be flexible, but not elastic. The midfoot ropes may be wrapped around the midfoot joint outer sheaves, and may pass through the upper midfoot rope hole and the lower midfoot rope hole. The midfoot joint outer sheaves may have a conical draft to them, for example, the ends may be smaller than the bases. If there were no other members to this joint, the joint may tend to disassemble itself, as the midfoot ropes may slide to the end of, and off of, the midfoot joint outer sheaves. To stop this from happening, the midfoot tightening rope may pass through the tightener rope hole on the forefoot and the heel. One end of this tightening rope may end in an adjustable screw. Specifically, the swage may have a female thread on the end, and the bolt may insert through the back of the heel. This screw may be used to tighten the joint, ensuring that it may not disassemble. In fact, this screw and the tightening rope may pull the midfoot ropes down, for example, onto the conically shaped midfoot outer sheaves. The tension may be kept reasonably low, for example, so the joint may not be too stiff, but there may be definite traction.

When the pylon advances toward the forefoot, the midfoot joint may begin to extend, stretching the midfoot actuators, bending the tightening rope, twisting the midfoot ropes, and tracking such that the midfoot joint attachment may move into (but may not touch) the movement clearance. This motion may continue, building force in the midfoot actuators, until the midfoot ropes reach their range of motion, and the upper midfoot rope hole may be suspended in the movement clearance.

When the forces on the pylon, or the forefoot decrease, the midfoot joint may flex back to its “neutral” conformation.

In operation, when the weight may be transferred from the heel to the forefoot, if not for the action of the midfoot tightener rope, the joint may slip apart. Since this action may be blocked, the heel may pivot up, suspended by the twist rope. The twist rope may transfer the weight to the forefoot. The rear weight platform may prevent the forefoot from “bucking” suddenly, rocking back so that the toe supports lift off of the ground. The heel may continue to rotate around the twist rope, increasing the twist. Due to the action of twisting the ropes and/or stretching the actuators, the twist may be increased with increasing difficulty, requiring more weight to advance further, resisting the twisting. This may allow amputees to relax into the walking motion, rather than using their quadriceps to control their motion over the foot. When the foot may be allowed to return to the flat configuration the rope may untwist, releasing energy/pulled in the plantarflexion direction by the actuators. The plantarflexion range of motion may be stopped by the plantarflexion limit rope wrapping around the plantarflexion limit rope sheave. The plantarflexion limit rope sheave may be supported by the sheave structural support. The plantarflexion limit rope may be guided by the plantarflexion limit rope guides, ensuring that it may not slip off the top of the midfoot attachment.

For the foot, beginning with a “fully flat” conformation, with all plantar surfaces on the ground, as one might find in single-leg stance phase, weight may be mostly on the heel.

As the pylon begins to tilt forward, the heel may rise and weight may be transferred to the ropes of the midfoot (subtalar) joint. Tension of the midfoot tightening rope may prevent the round midfoot sheaves from slipping out of the midfoot ropes. As the heel rises, the weight may be born by the midfoot ropes and the round midfoot sheaves, suspending the compression members from the tension members; a tensegrity structure.

When the midfoot joint reaches its maximum dorsiflexion of about 10-15 degrees, the dorsiflexion may be stopped by the midfoot ropes, and the forefoot may begin to lift off of the ground. The platform in the back of the forefoot may rise, and the weight may be now borne by the angled walking surfaces which may be analogous to the balls of the first and fifth toes. These surfaces may be of different radii, for example, mimicking the shape of the human foot. If the forefoot were allowed to rotate with no further guidance, it may roll to the lateral side, for example, toward the smaller radius walking surface.

The function of the lateral, smallest toe may be relevant. As the forefoot rotates, the toes may remain in contact with the ground, for example, rotating around the axial rope. After about 5-15 degrees of rotation (the exact number may be tuned to the individual's needs and the geometry of their device), the little toe's motion may be blocked by its dorsiflexion limit rope. This may be relevant because, as the lateral walking surface of the forefoot lifts off the ground, the walking surface of the little toe may now become a primary weight bearing surface. The walking surface of the little toe may be farther from the center of rotation than the walking surface of the medial forefoot, so the roll of the forefoot may be now directed medially. This may be a more anatomically correct direction.

These lateral and medial rolling motions may not be transmitted to the tibial shaft or the amputee's residuum. For example, their motion may be expressed at the midfoot joint as movement between the round midfoot sheaves and the forefoot, subtly changing the orientations of the midfoot joint ropes. Similar rotations and counter rotations may be seen in gait motion analysis of the feet. Thus, the amputee's comfort may be maintained and greater walking stability, through guided motion, may be achieved.

The middle and great toes may continue to stabilize the motion by the slight back pressure of their actuators, for example, opposing the motion. Thus, the amputee may not just “fall forward” during his gait, but may be guided with proper timing.

When the great toe (and optionally the middle toes) reach a critical angle, again tuned by the needs of the user, the motion coupling rope may be pulled taught. This rope may connect the toes with the hindfoot, spanning the MTP joints and the midfoot (subtalar) joint, and may be anatomically analogous to the peroni, the plantar fascia and other muscles. As the toes may be forced into dorsiflexion by the body weight, they may pull the midfoot joint back into its “flat” conformation. This movement may rotate the tibial shaft, slightly extending the knee, beginning toe off and swing phase. Thus, the foot may begin a swing phase, reducing or eliminating the need for the amputee to forcefully swing the leg out from the knee and quadriceps, a classic “smart” gait compensation that may have been linked to early joint degeneration for many amputees.

After this motion, the toes (excepting the little toe) may continue to roll forward and encounter their dorsiflexion limit rope. This may end all motion of the foot and toe off may then be the next gait phase.

Heel strike on the cushioned heel may be the next movement involving the device, followed by flat foot, and then the cycle may repeat with each ensuing step.

For the ankle, the ankle may be a tensegrity joint that may be constrained to move in certain planes of motion and not others. The heel attachment stubs may be on the medial and lateral (side to side) sides of joint. The U-shaped end of the lower leg (lower joint member) may run in the ante-posterior direction (front to back). The lower joint member may be suspended from the heel attachment stubs by the ankle universal rope. The ankle stabilizer ropes may constrain the medio-lateral rotation of the ankle joint. The ankle stabilizer ropes also may prevent the heel attachment stubs and the U-shaped end of the lower leg from moving away from each other. Furthermore, the joint may be compressed and stabilized by the ankle stabilizer ropes. These ropes may be tight, pulling the lower joint member toward the heel. This tension may be opposed by the ankle universal rope. This stored tension between the ankle stabilization ropes and the ankle universal rope may be adjusted by changing the length of the stabilizer ropes. This may be accomplished by use of adjustable swages, in accordance with the art of wire rope design. This tension may control the stiffness of the joint. More tension may equal more stiffness. The two ankle stabilization ropes may be adjusted independently, which may allow for different amounts of angular movement (e.g., different ranges of motion) in the medial direction or the lateral direction. This may be relevant for mimicking the natural human ranges of motion of the ankle joint.

Generally, motion may be allowed by the ankle universal rope, for example, as it bends around the sheaves on the U-shaped end of the lower leg and the sheaves on the heel attachment stubs. Most of this motion may be in the ante-posterior direction.

The ankle limit rope and the u-shaped ankle limit rope may constrain the rotational motion of the ankle joint in the anterior (e.g., forward) and posterior (e.g., rearward) directions, respectively. In order to alter the torque/angle response curve of this motion, actuators (e.g., elastic members) may be added to or used to replace the ankle limit rope and/or the u-shaped ankle limit rope.

Twisting of u-shaped end of the lower leg relative to the heel attachment stubs may be also blocked by this geometry, although there may also be an individual tensile member that accomplishes this task. This twisting may engage the ankle stabilizer ropes, pulling the joint members closer to each other. This motion may be blocked by the universal joint rope. Thus, the joint members slipping on the U-rope, twisting relative to each other may be loosely blocked by this geometry. This “loose” blocking may be relevant for prosthetic ankle applications, as the natural anatomy may have a similar compliance. In the embodiment shown, this twist may be blocked in one direction more than the other. Another ankle stabilizer rope may be added to further impede this motion.

For the knee, the knee may be a tensegrity joint that may be principally constrained in the medio-lateral (e.g., side to side) direction. The upper knee universal rope and the lower knee universal rope may both be mounted on the x-brace. These two ropes may also connect to the upper joint member and the lower joint member, respectively. The upper knee universal rope and the lower knee universal rope may stop the joint from collapsing under load, and they also may provide support for the motion of the joint. The upper knee stabilization ropes and the knee tightener rope may constrain the medio-lateral motion. The knee tightener rope may also block the spreading apart of the joint. The lower knee stabilization ropes may function in the same manner.

The knee tightener rope may be taught, compressing the joint structure. This compression may stabilize the knee joint, for example, making it more stable and/or less likely to twist. Too much compression may make the knee too stiff and may damage the ropes. Too little compression may make the joint unstable.

The radii of the u-shaped end of the upper joint member and the u-shaped end of the lower joint member may be such that, when the knee bends, the smaller radius of the u-shaped end of the lower joint member may not interfere with the motion by contacting the u-shaped end of the upper joint member. None of the compression members (e.g., x-brace, upper joint member, lower joint member) may contact each other in normal service conditions.

These radii may be chosen, relative to the span of the x-brace (e.g., the distance between holes 516), so that the lowest energy configuration may be an orthogonal alignment of the unshaped end of the upper joint member relative to the x-brace. This may be also true of the u-shaped end of the lower joint member and the x-brace. The knee tightener rope may also re-enforce this rotational stability by holding the upper joint member universal rope holes lower than the upper pair of x-brace universal rope holes. The same may be true of the lower knee universal rope and the conformation of the lower joint member relative the x-brace.

Bending the knee may wrap the upper and lower universal ropes around the sheaves mounted on the x-brace, the upper joint member, and/or the lower joint members.

Rotational stability of the knee may be very similar to the ankle. The knee may use a split stabilizer rope scheme, making the rotational stability substantially symmetric.

Sheaves may be also used to protect the knee tightener rope.

All sheaves may have a round surface so that the ropes may be protected from kinking as the rope bends. However sheaves do not have to be strictly round. Though many simple cylindrical sheaves may be used in the drawings, in practical use, many sheaves may have edges to them that may guide the ropes.

A comparison of the cylindrical sheaves and sheaves with edges for guiding ropes may be shown in one or more of the figures. These edges may keep the rope from sliding off of the edge of the cylinder.

The height of the edges may determine the stability of the rope during loading and bending. The amount of curvature in the sheave relative to the radius of the rope may be determined in accordance with the art of wire rope design. About 60 degrees of supportive contact may be desirable.

Another example of this shaped sheave may be seen in the forefoot, at the intersection of limit rope guides 126 and dorsiflexion limit rope holes 116. A further example of the curved sheaves may be found on the toe distal toe sheave 302, side toe sheave 300, and/or the plantarflexion sheave surface 192.

Various actuators and their attachments may be placed relative to the centers of motion of the joints to produce certain torque response curves around those joints.

Materials for making compression and tension members useful in the practice of the invention may be sufficiently rigid, strong, flexible, as appropriate for the function of that member. The weight of the user and the selected use by that user may be considered when selecting materials. For example, stronger materials may be required when the user intends to jump out of an airplane compared to when the user merely intends to walk. Materials for making actuators useful in the practice of the invention may be appropriately strong and/or elastic. Useful materials for compression members may include metals and/or plastics. Useful materials for tension members may include steel wire rope. Metal and/or plastic bearings may be also useful in the practice of the invention. Preferably the compression and tension members of the joints and leg parts of the invention may be made from LiquidMetal (TM), Aluminum (7075 T6), steel wire rope, bearings, tool steel, and/or plastic.

Prototypes of prostheses, orthotics, and robots may be fashioned out of wood, brass, aluminum, plastic, yarn, and steel wire rope. Almost any material having the appropriate characteristics may be used, as long as they do not interfere with the function of the joint or the experience of the user.

The invention may provide artificial tensegrity joints for prosthetic, orthotic, and robotic devices for skeletal animals comprising at least two compression members connected by at least one tension member. Optionally the joint also may comprise at least one actuator connecting the two compression members. The joints of the invention may have a similar range of motion as the equivalent natural joints. Joints provided by the invention include, but may not be limited to foot joints, ankle joints, knee joints, or hip joints, midfoot joints and metatarso-phalangeal (MTP) joints. The invention may provide artificial feet, legs, and foot and leg portions.

In embodiments of the invention, at least one compression member may be artificial. The joint may be weight-bearing joint. The joint may be for a human. The joint may have similar or better strength as the equivalent natural joint. The joint may be functionally similar to the equivalent natural joint. The joint may be a lower extremity joint, may be a polycentric joint, may be a universal joint, may comprise two or more universal joints, may comprise two or more universal joints stacked on top of each other, may be a non-shoulder joint, may be for improving ambulation of the animal, and/or may be for a non-human animal.

The invention may provide prosthetic, orthotic, and robotic devices for skeletal animals wherein the device may comprise an artificial tensegrity joint.

The invention may provide devices having one or more artificial tensegrity joints having a range of motion similar to an equivalent natural joint of the animal. Various embodiments may include one or more of any of the aspects set forth herein. The ambulation of the skeletal animal may be improved compared to the animal without the device. The skeletal animal may be mature or immature, may include a foot, an ankle, a knee, a hip, and/or a leg, may include an MTP joint or a midfoot joint, or both, may be a powered lower body orthotic, and/or the animal may be a human.

The invention may provide prosthetic for a lower extremity joint of an animal. The device may be of a weight selected to form an artificial leg and/or combined artificial and natural leg that may be substantially equal to the weight of the paired leg of the animal.

The invention may provide a plurality of tensegrity joints for a prosthetic, orthotic, or robotic device for a skeletal animal. Each joint may include at least two compression members connected by at least one tension member.

In an embodiment of the invention, the plurality of tensegrity joints may together form one or more of a foot; knee; foot and ankle; foot, ankle, and knee; a complete leg; or a complete leg and at least a portion of a hip.

The invention may provide at least a portion of a tensegrity joint for a prosthetic, orthotic, or robotic device for a skeletal animal including at least one artificial compression member connected by at least one tension member, and a means for connecting the tension member to a second artificial or natural compression member.

The invention may provide at least a portion of a tensegrity joint for a prosthetic, orthotic, or robotic device for a skeletal animal including at least one tension member and means for connecting the tension member to two or more artificial or natural compression members.

The invention may provide at least a portion of an artificial midfoot joint including one or more of the following aspects: a midfoot joint rope; a midfoot tightener rope; a forefoot; and a means for attaching the ropes to a heel and the forefoot. The ropes may connect the heel and forefoot in a tensegrity joint that may have a range of motion similar to a natural midfoot joint portion.

The invention may provide an artificial midfoot joint comprising one or more of the following aspects: a midfoot joint rope; a midfoot tightener rope; a heel; a forefoot; and a means for attaching the ropes to the heel and forefoot. The ropes may connect the heel and forefoot in a tensegrity joint that may have a range of motion similar to a natural midfoot joint.

The artificial midfoot joint also may include a means for attaching the heel to an ankle joint or leg. The artificial midfoot joint also may include a midfoot actuator connecting the heel and the forefoot.

The artificial midfoot joint also may include a component which may be one or more of a twist rope, a plantarflexion limit rope, a plantarflexion limit rope sheave, a plantarflexion limit rope guide, a round, midfoot outer sheave, a tapered midfoot joint outer sheave, a cushion cavity, a coordination rope attachment, and angled plate mount for sheaves. The invention may optionally include means for attaching each of the components to the heel and/or forefoot.

The artificial midfoot joint may be a midfoot twist joint. Various embodiments may include one or more of the following aspects: a twist rope; a plantarflexion limit rope; a plantarflexion limit rope sheave; a plantarflexion limit rope guide; a pair of round midfoot outer sheaves; a midfoot twist tapered midfoot joint outer sheaves; a midfoot twist cushion cavity; a midfoot twist coordination rope attachment; and an angled plate for the sheaves.

The invention may provide at least a portion of an artificial MTP joint comprising one or more of the following aspects: an axial rope; an dorsiflexion limit rope; a plantarflexion limit rope; one or more toes; and a means for attaching the ropes to the toes and a forefoot. The ropes may connect the toes and the forefoot in a tensegrity joint that may have a range of motion similar to a natural MTP joint portion.

The invention may provide an artificial MTP joint comprising one or more of the following aspects: an axial rope; an dorsiflexion limit rope; a plantarflexion limit rope; one or more toes; a forefoot; and means for attaching the ropes to the toes and forefoot; wherein the ropes connect the toes and forefoot in a tensegrity joint that may have a range of motion similar to a natural MTP joint.

The artificial MTP joint also may include a means for attaching the forefoot to a midfoot joint or heel. One or more toe actuators may connect each of the toes and the forefoot. Embodiments of the invention may include a component including one or more of screw swages, stop swages, a stop swage keeper, a bottom rope keeper, and rope guide keepers. Embodiments of the invention may optionally include a means for attaching the component(s) to the toes and/or the forefoot.

The invention may provide at least a portion of an artificial tensegrity prosthetic, orthotic, or robotic foot for a human comprising one or more of the following aspects: one or more toes; a forefoot; one or more rope tension members for forming a tensegrity MTP joint between the toes and the forefoot; and one or more rope tension members for forming a tensegrity midfoot joint between the forefoot and a heel. The foot portion may have a range of motion similar to an equivalent natural human foot portion.

The invention may provide an artificial tensegrity prosthetic, orthotic, or robotic foot for a human comprising one or more of the following aspects: one or more toes; a forefoot; a heel; one or more rope tension members for forming a tensegrity MTP joint between the toes and the forefoot; and one or more rope tension members for forming a tensegrity midfoot joint between the forefoot and the heel. The foot may have a range of motion similar to a natural human foot.

Various embodiments of the foot or other portions may include one or more of the following aspects: an axial rope; an dorsiflexion limit rope; a plantarflexion limit rope; means for attaching the axial rope, dorsiflexion limit rope, and plantarflexion limit rope to the toes and forefoot; a midfoot joint rope; a midfoot tightener rope; and means for attaching the midfoot joint rope and midfoot tightener rope to the heel and forefoot.

The foot may include one or more toe actuators connecting each of the toes and the forefoot and/or a midfoot actuator connecting the heel and the forefoot.

The foot may include component(s) including one or more of screw swages, stop swages, stop swage keeper, a bottom rope keeper, rope guide keepers, a twist rope, a plantarflexion limit rope, a plantarflexion limit rope sheave, a plantarflexion limit rope guide, round, midfoot outer sheaves, tapered midfoot joint outer sheaves, a cushion cavity, a coordination rope attachment, and angled plate mount for sheaves. Embodiments of the invention may optionally include a means for attaching the components to the toes and/or the forefoot and/or means for attaching each of the component(s) to the heel and/or forefoot.

Various embodiments, some of which may include the foot, may include component(s) including one or more of the following aspects: toe supports; front sheaves; an axial rope hole; a tall axial sheave; a short axial sheave; an axial sheave rope keeper flange; a spacer for plantarflexion ropes; a short spacer; dorsiflexion limit rope holes; open dorsiflexion limit rope holes; plantarflexion limit rope holes; open plantarflexion limit rope holes; dorsiflexion limit rope guides; limit rope guides; a plantarflexion limit rope guide trim; a plantarflexion limit rope spreader; rope guide fillets; an axial rope hole cleanup; a dorsiflexion limit rope spreader; dorsiflexion limit rope spreader cuts; spreader bar cuts- plantarflexion ropes; a rollover profile; fillets; stiffener bars; a virtual axial load platform; a medial angled walking surface; a lateral angled walking surface; a screw attachment bar; screw attachment ribs; a midfoot joint attachment; a clearance for a midfoot joint; a first midfoot rope hole; a second midfoot rope hole; forefoot actuator stays; a tightener rope hole; a rear weight platform; an axial rope screw hole attachment; an axial rope swage loop attachment hole; a first midfoot attachment hole; a second midfoot attachment hole; a plantarflexion limit rope sheave; a sheave structural support; a plantarflexion limit rope guide; and a plantarflexion sheave surface.

Various embodiments, some of which may include the foot, may include component(s) including one or more of the following aspects: a cushioned heel; midfoot joint outer sheaves; a Movement Clearance; a pylon; a tightener rope hole; a heel actuator stay; heel alt—tapered midfoot joint outer sheaves; a heel cushion cavity; a coordination rope; a heel coordination rope attachment; an angled plate; ankle limit rope holes; a side toe sheave; a distal toe sheave; an angled walking surface; a main column; a cleanup; an axial rope hole; an axial rope hole cleanup; an actuator hook; a front sheave fillet; an upper fillet; a lower fillet; a front actuator hook fillet; a rear actuator hook fillet; an inner hook fillet; a plantarflexion limit rope toe sheave; a plantarflexion limit rope toe sheave back fillet; a Bearing Clearance; a thick web; a thin web; a top beam; a Thin Web; a top beam; a toe race; an inner curve; and bearings.

Various embodiments, some of which may include the foot, may include one or more of the following aspects: a means for attaching the heel to an ankle joint or leg; a means for attaching the foot to a leg of the human; two or more tension members for each of the MTP joint and midfoot joint; two or more toes; toes of different sizes; one big toe on the proximal side of the foot; one big toe, one or more medium toes, and one small toe, ordered from proximal to distal, respectively.

Various embodiments may include means for attaching the foot to the leg of a human may comprise an artificial tensegrity ankle joint.

Various embodiments may include one or more of the following aspects: the heel may be cushioned; the use of an embodiment of the prosthetic foot described herein may improve ambulation of a the human more than one or more of a conventional prosthetic foot (CF), a solid ankle cushioned heel foot (SACH), or an energy storing foot (ESF); the use of an embodiment of the device described herein may increase the stride length of the human, which may result in less oxygen consumption by the human and/or decrease the impact force of the heel strike of an intact foot of the human; an embodiment of the device described herein may increase ankle plantarflexion power on toe off compared to a CF, SACH, or ESF; using an embodiment of the device described herein may ease ambulation, may reduce injuries, or both compared to using a CF, SACH, or ESF; use of an embodiment of the device described herein may result in a stride that rolls over the prosthetic foot closer to the MTP joint and further from an ankle joint attached to the foot compared to use of a CF, SACH, or ESF; use of an embodiment of the device described herein may result in about half of the angular change between the lower leg attached to the foot and the floor results from motion at the midfoot joint; use of an embodiment of the device described herein may result in less heel rise before fully dorsiflexing compared to use of a CF, SACH, or ESF.

The invention may provide at least a portion of an artificial ankle joint including one or more of the following aspects: an ankle universal rope; a heel having a universal attachment stub; and means for attaching the rope to the heel and a u-shaped end of a lower leg portion. The rope may connect the heel and lower leg portion in a tensegrity joint that may have a range of motion similar to a natural ankle joint.

The invention may provide an artificial ankle joint including one or more of the following aspects: an ankle universal rope; a heel having a universal attachment stub; a lower leg portion having a unshaped end; and means for attaching the rope to the heel and u-shaped end of the lower leg portion. The rope may connect the heel and lower leg portion in a tensegrity joint that may have a range of motion similar to a natural ankle joint.

The artificial ankle joint may comprise a means for attaching the leg to a second portion of a lower leg or a knee joint. Embodiments of the invention may include one or more components including one or more of the following: an ankle stabilizer rope; an ankle limit rope; a u-shaped ankle limit rope; means for attaching each of the aforementioned limit ropes to the heel and the lower leg portion; heel universal rope holes; heel stabilizer rope holes; lower leg universal rope holes; lower leg stabilizer rope holes; lower leg limit rope holes; and sheaves with a means for attaching sheaves to the heel or the lower leg portion.

The invention may provide at least a portion of an artificial knee joint including one or more of the following aspects: an x-brace; one or more knee universal ropes; a means for attaching the knee universal rope to a u-shaped end of a proximal leg portion or a u-shaped end of a distal leg portion; means for attaching the ropes to the x-brace. The ropes may be capable of connecting the x-brace and proximal or distal leg portions in a tensegrity joint portion that may have a range of motion similar to an equivalent portion of a natural knee joint.

The invention may provide an artificial knee joint including one or more of the following aspects: an x-brace; a first knee universal rope; a second knee universal rope; a means for attaching the first knee universal rope to a u-shaped end of a proximal leg portion; a means for attaching the second knee universal rope to a u-shaped end of a distal leg portion; and means for attaching the ropes to the x-brace. The ropes and x-brace may be capable of connecting the proximal and distal leg portions in a tensegrity joint that may have a range of motion similar to a natural knee joint.

The invention may provide an artificial knee joint including one or more of the following aspects: an x-brace; an first knee universal rope; a second knee universal rope; a distal leg or distal leg portion having a u-shaped end; a proximal leg portion or proximal leg having a u-shaped end; and means for attaching the ropes to the x-brace and the distal leg or proximal leg portion. The ropes may be capable of connecting the proximal leg or leg portion, x-brace, and distal leg or leg portion in a tensegrity joint that may have a range of motion similar to a natural knee joint.

The artificial knee joint may include one or more of a means for attaching the proximal leg to a hip joint or leg portion to a second portion of a proximal leg; one or more proximal knee stabilization ropes and/or distal knee stabilization ropes; and means for attaching each to at least two of the x-brace; distal leg; and proximal leg portion. Embodiments of the invention may include one or more of the following: x-brace universal rope holes; x-brace stabilization rope holes; upper joint member universal rope holes; lower joint member universal rope holes; upper joint member stabilization rope holes; lower joint member stabilization rope holes; a knee tightener rope; an x-brace tightener rope hole; an upper joint member tightener rope hole; a lower joint member tightener rope hole; and sheaves with a means for attaching sheaves to the x-brace, distal leg or proximal leg portion, and/or two universal joints stacked on top of each other.

The invention may provide an artificial prosthetic, orthotic, or robotic distal leg portion or leg including one or more of the following aspects: a tensegrity MTP joint, a tensegrity midfoot joint, a tensegrity ankle joint, at least a portion of a tensegrity knee joint, and a means for connecting the leg or leg portion to a proximal leg portion, a hip, or a pelvis.

The invention may provide a method for ambulating with a prosthetic, orthotic, or robotic foot. The foot may include a midfoot joint or an MTP joint. The method may include bending the foot at the midfoot joint or the MTP joint. The midfoot joint or MTP joint may be a tensegrity joint. The bending the foot at the MTP joint may result in toe off.

The method may including bending the foot at both the midfoot joint and the MTP joint and/or bending the foot at a plurality of MTP joints.

A human may be ambulating. The method may including ambulating with one prosthetic, orthotic, or robotic feet. The method may result in more symmetrical walking than using a CF or an ESF.

The invention may provide a method for ambulating using a prosthetic, orthotic, or robotic foot. The method may include performing a toe off step.

The invention may provide a method for ambulating including bending a prosthetic, orthotic, or robotic tensegrity joint. The tensegrity joint may include one or more of an MTP joint, a midfoot joint, an ankle joint, a knee joint, and a hip joint.

The method may include one or more of: bending a prosthetic, orthotic, or robotic tensegrity midfoot joint; and bending a prosthetic, orthotic, or robotic tensegrity MTP joint.

Various embodiments may include one or more of the following aspects: bending a prosthetic, orthotic, or robotic tensegrity ankle joint; bending a prosthetic, orthotic, or robotic tensegrity distal knee joint portion; bending a prosthetic, orthotic, or robotic tensegrity proximal knee joint portion; and/or bending a prosthetic, orthotic, or robotic tensegrity hip joint.

The invention may provide a method of bending a prosthetic, orthotic, or robotic joint comprising one or more of the following aspects: applying force to a first compression member; applying tension to a tension member; and thereby applying a force to a second compression member.

In an ankle joint, as may be known in the art, anterior/posterior movement may be good, but medial/lateral movement may be bad. Additional ankle ropes may be used to provide more lateral stability. Various embodiments may include two or more universal tensegrity joints.

### EXAMPLE 1

The prototype foot illustrated in FIGS. 1-10 may have been built using wood, brass, aluminum, plastic, yarn, and/or steel wire rope. It may be a right foot and may have one big toe, two medium toes, and one small toe.

### EXAMPLE 2

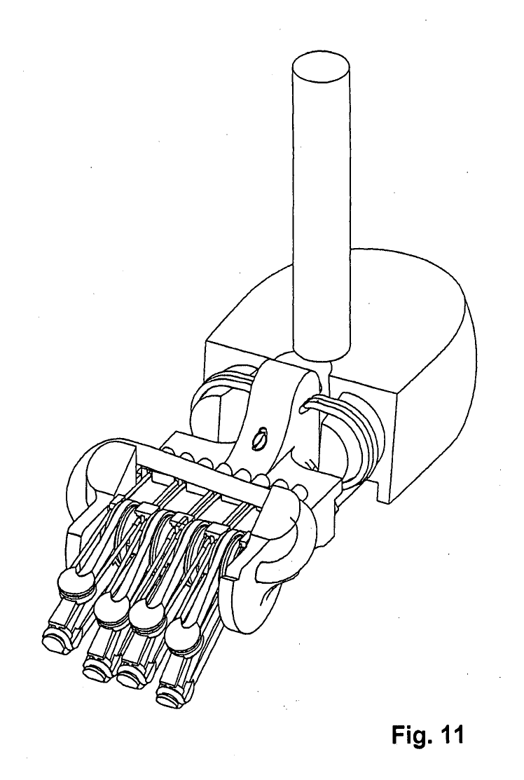

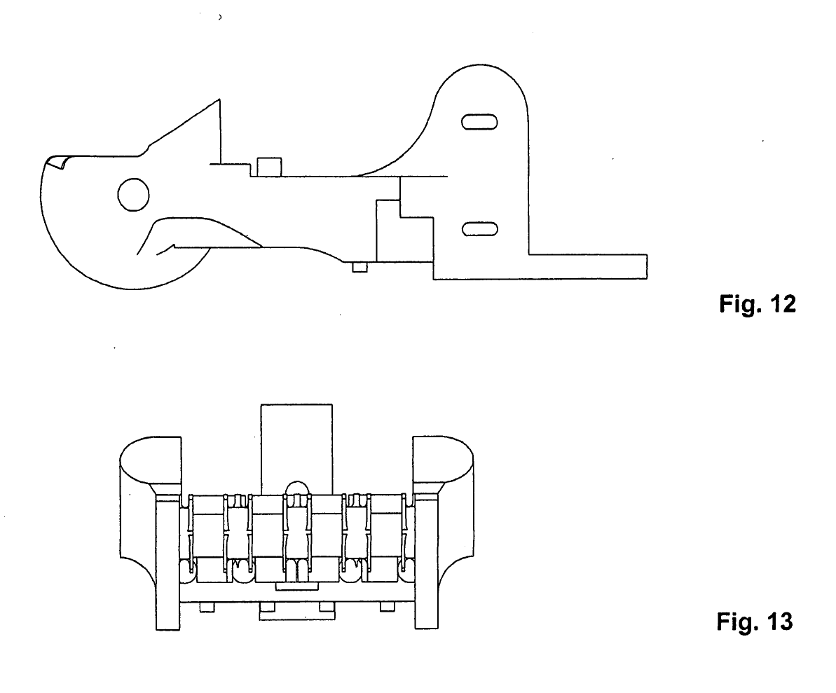

The prototype orthotic boot illustrated in FIGS. 11-13 may have been built from wood, brass, aluminum, plastic, yarn, and/or steel wire rope cable. Several parts may be different from the foot illustrated in FIGS. 1-10. The boot may have four large toes, the midfoot joint attachment may be at about a 90-degree angle with the rest of the forefoot, and the parts on the outsides of the toes may be shorter.

### EXAMPLE 3

The prototype ankle illustrated in FIGS. 14-17 may have been built using wood, brass, aluminum, plastic, yarn, and/or steel wire rope.

### EXAMPLE 4

The prototype knee illustrated in FIGS. 20-22 may have been built using wood, brass, aluminum, plastic, yarn, and/or steel wire rope.

### EXAMPLE 5

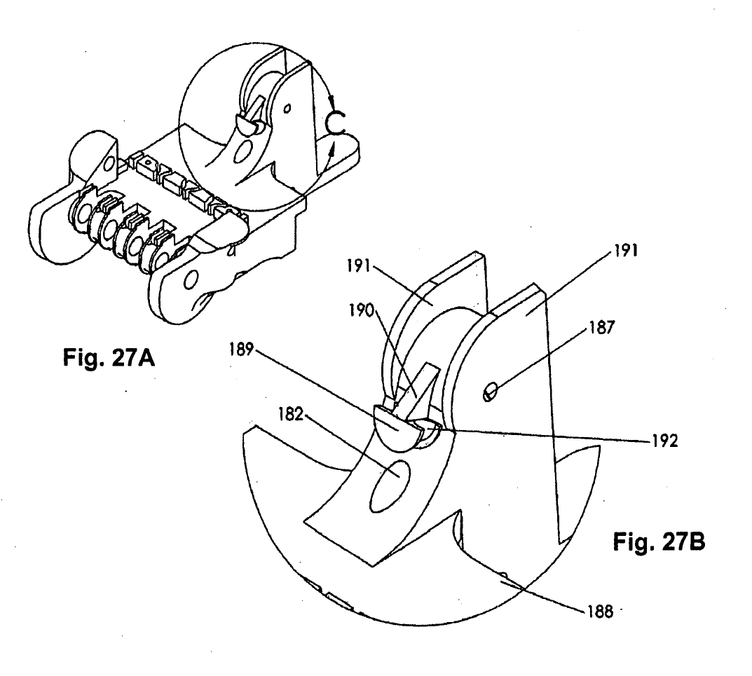

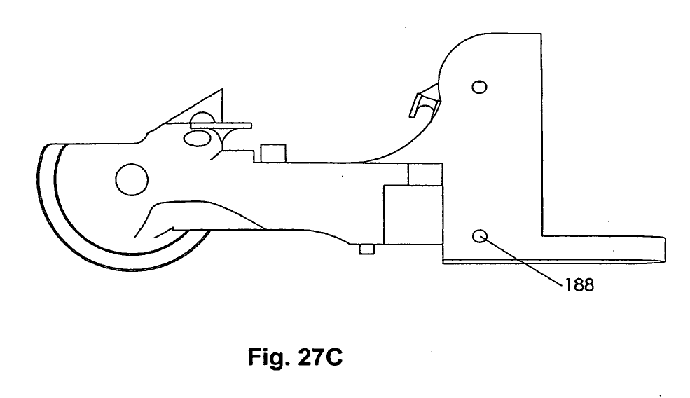

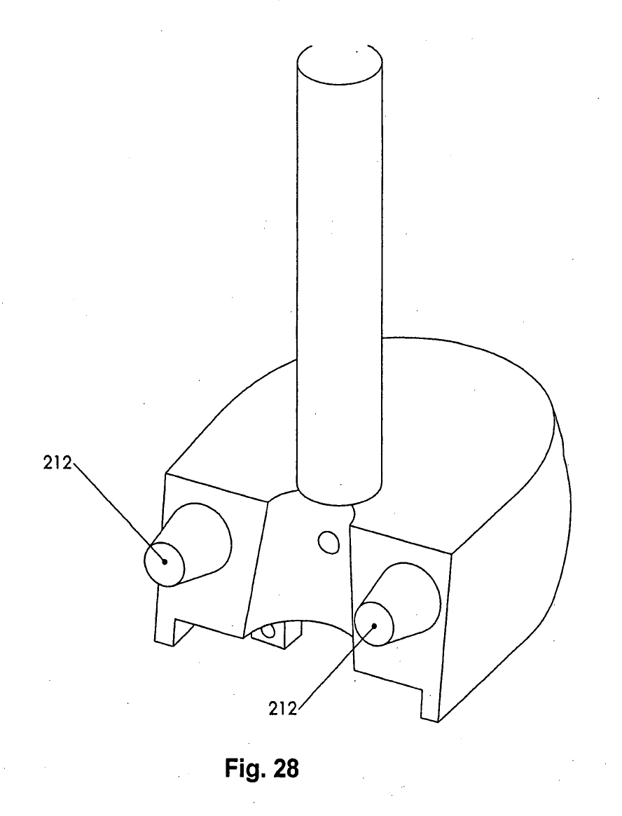

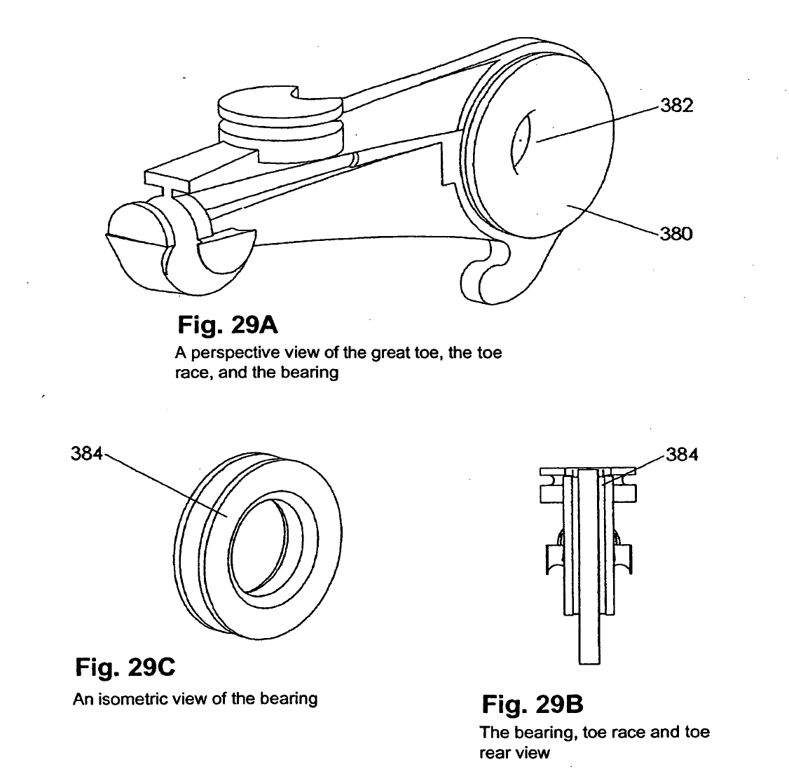

The prototype midfoot twist illustrated in FIGS. 27-29 may have been built using wood, brass, aluminum, plastic, yarn, and/or steel wire rope.

### EXAMPLE 6

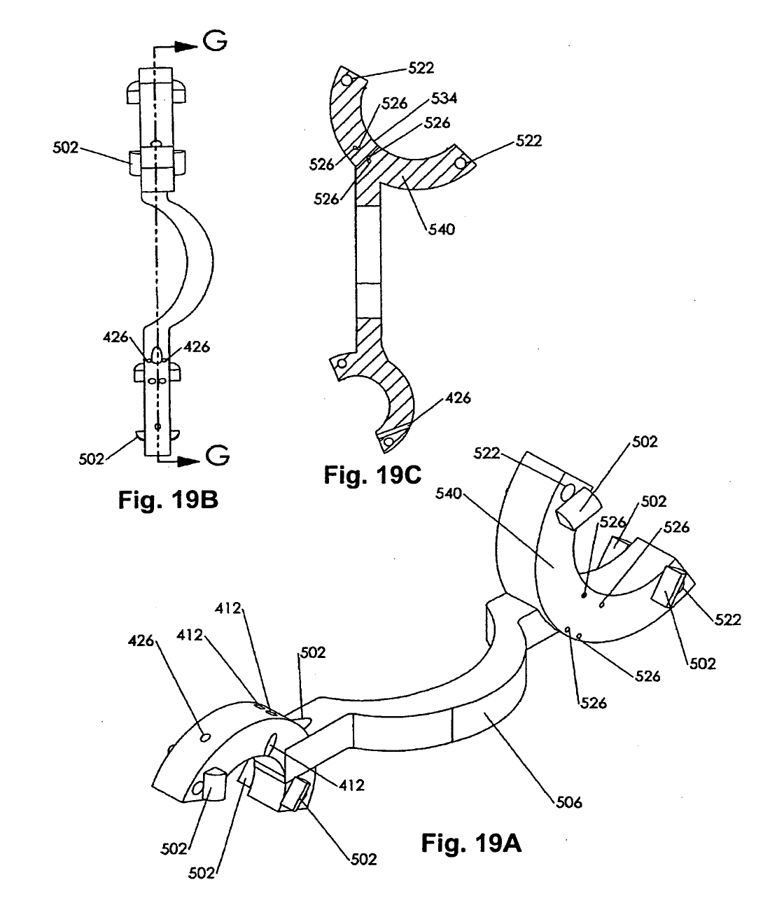

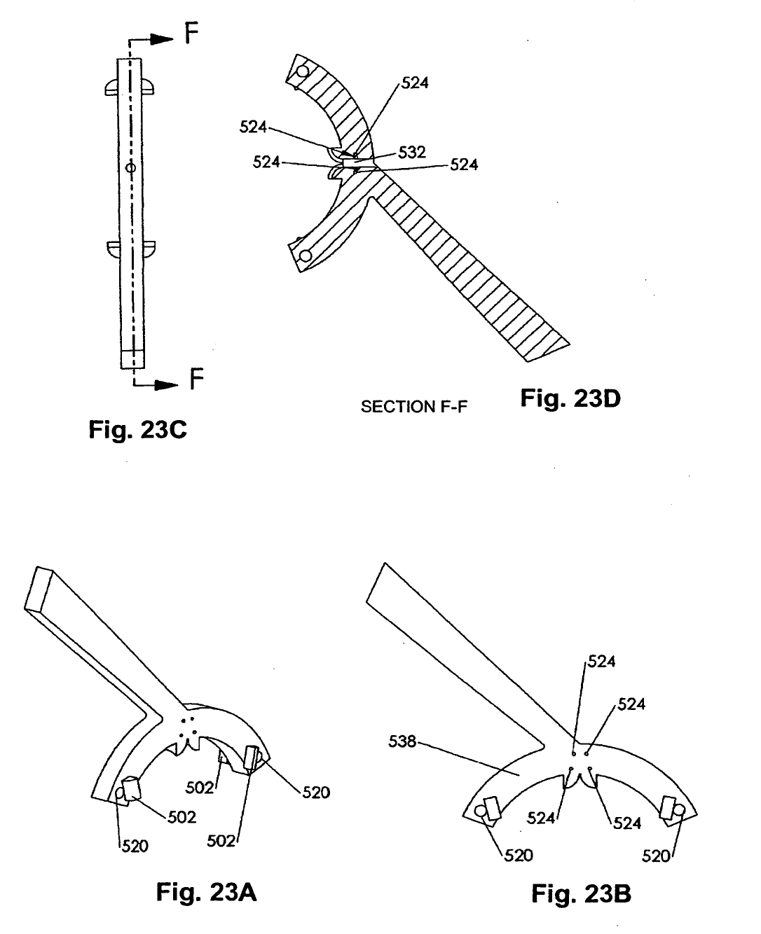

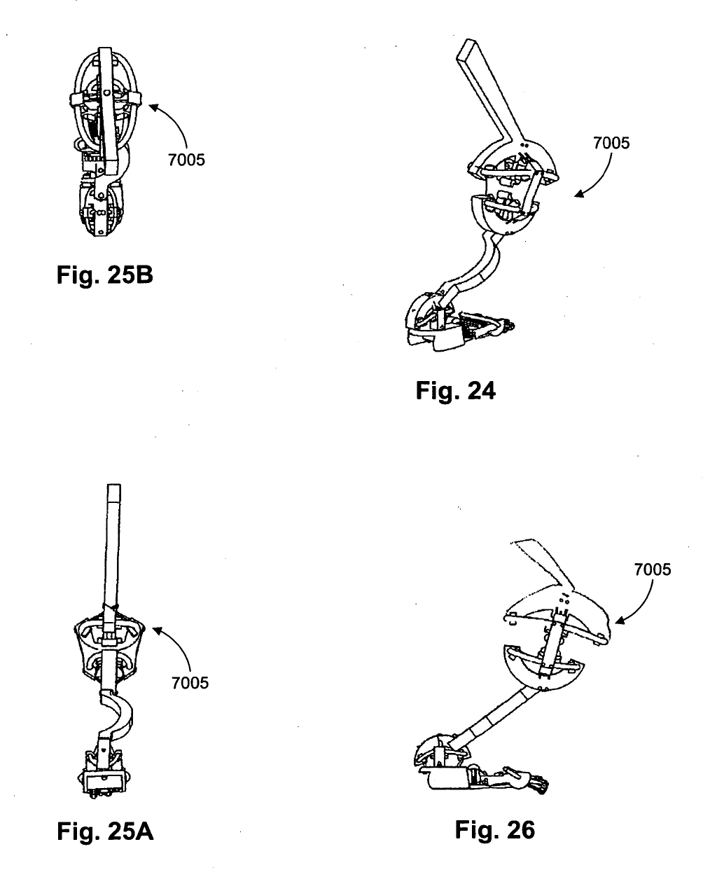

The prototype leg illustrated in FIGS. 19 and 23-26 may have been built using wood, brass, aluminum, plastic, yam, and/or steel wire rope.

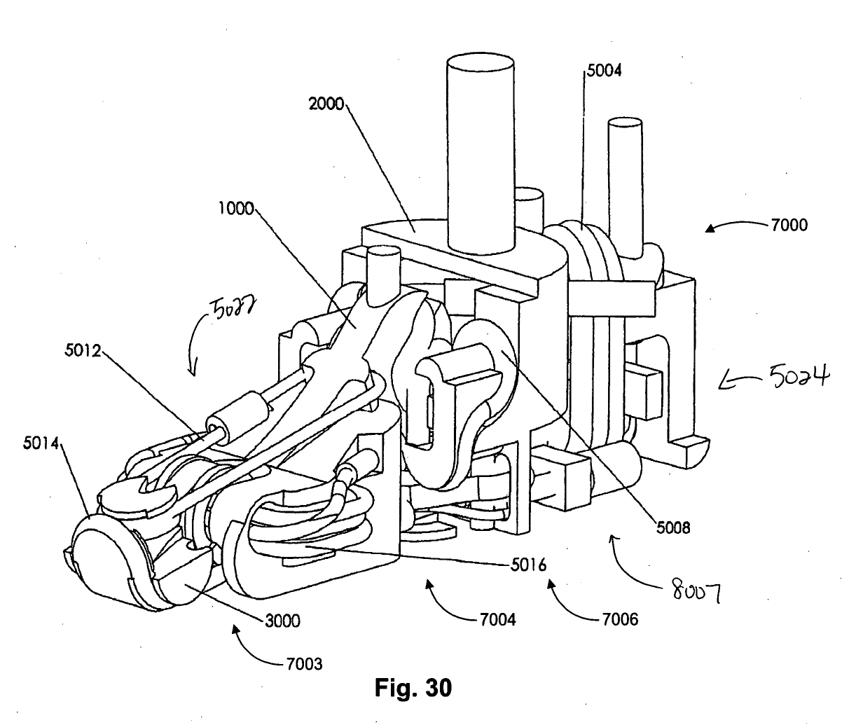

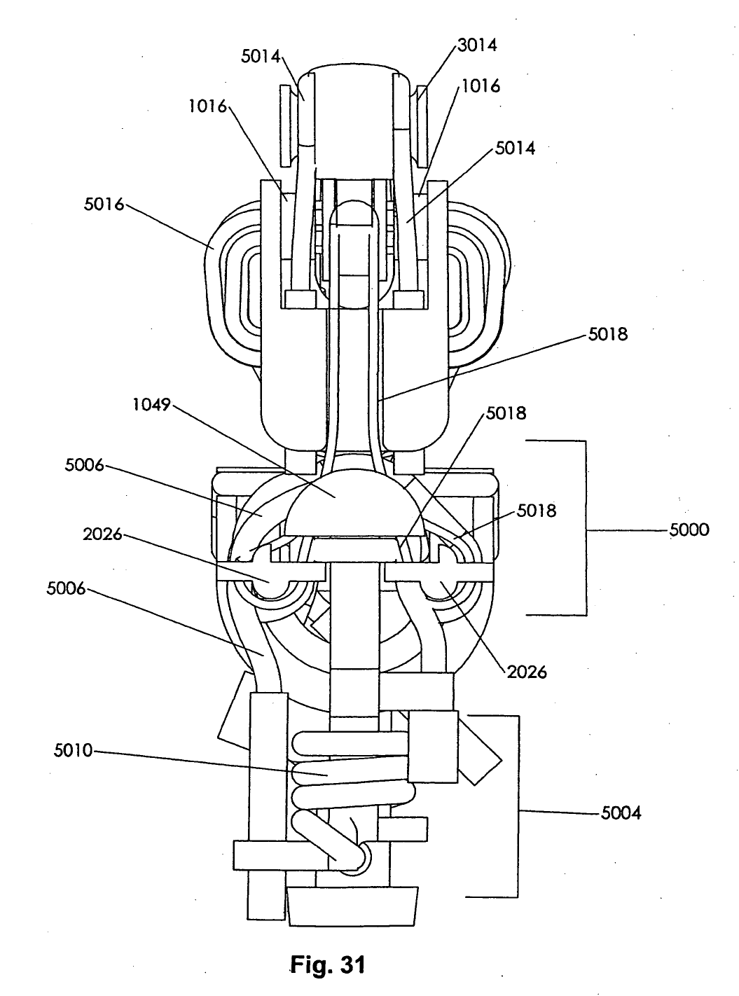

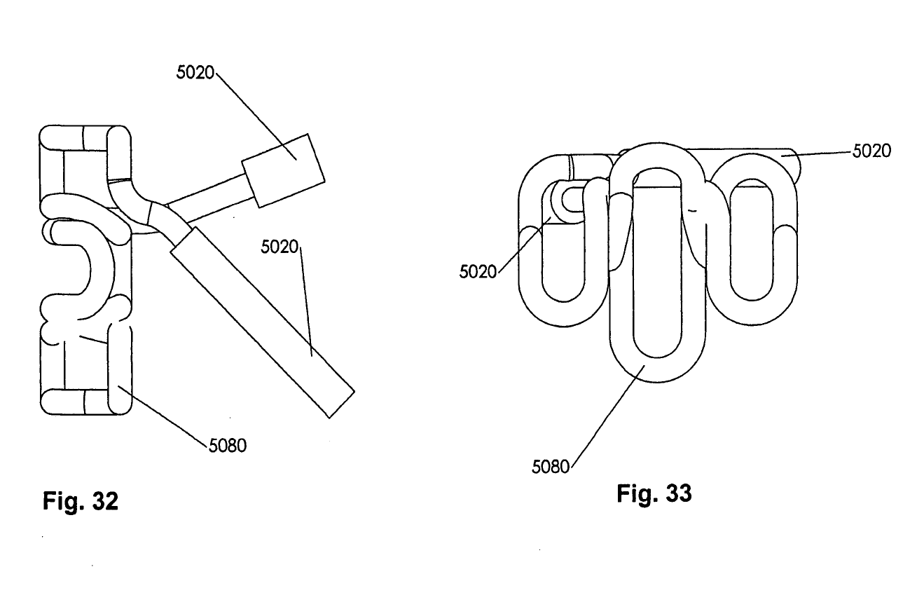

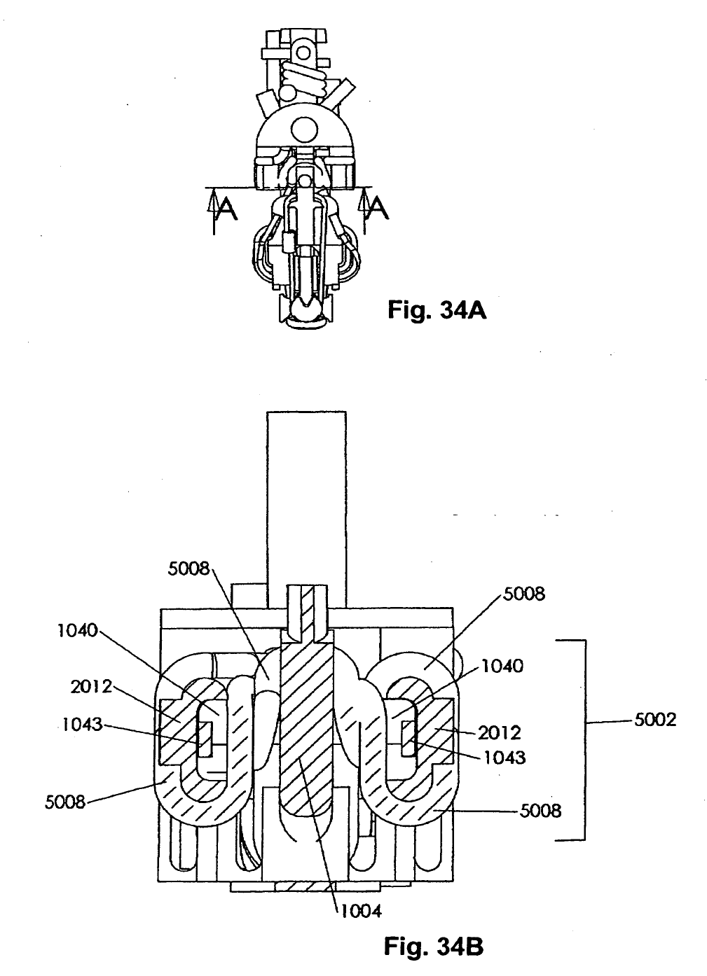

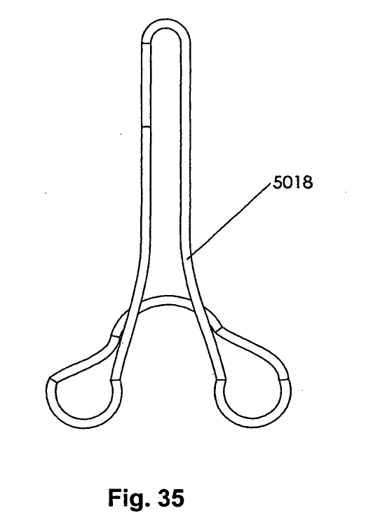

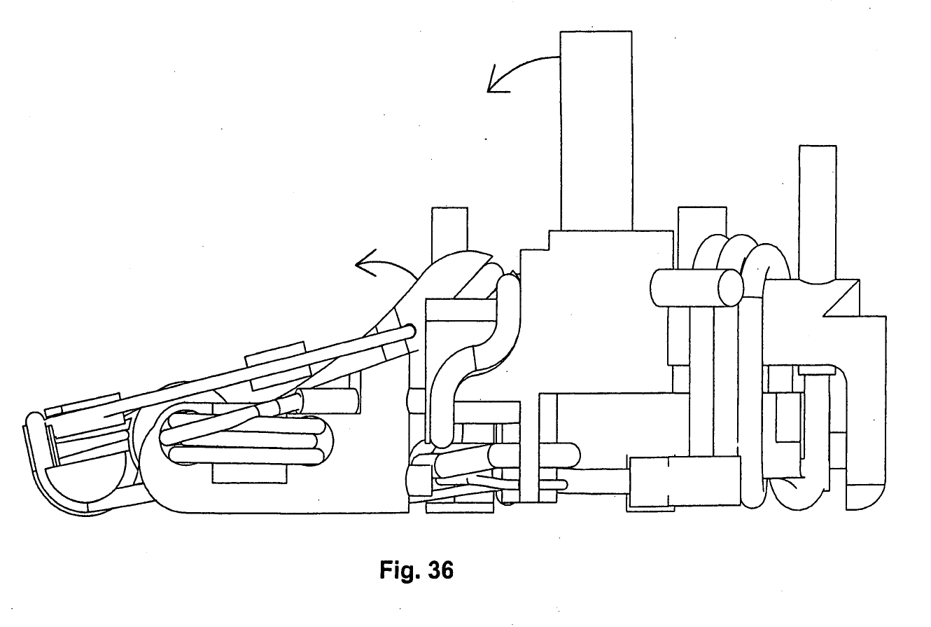

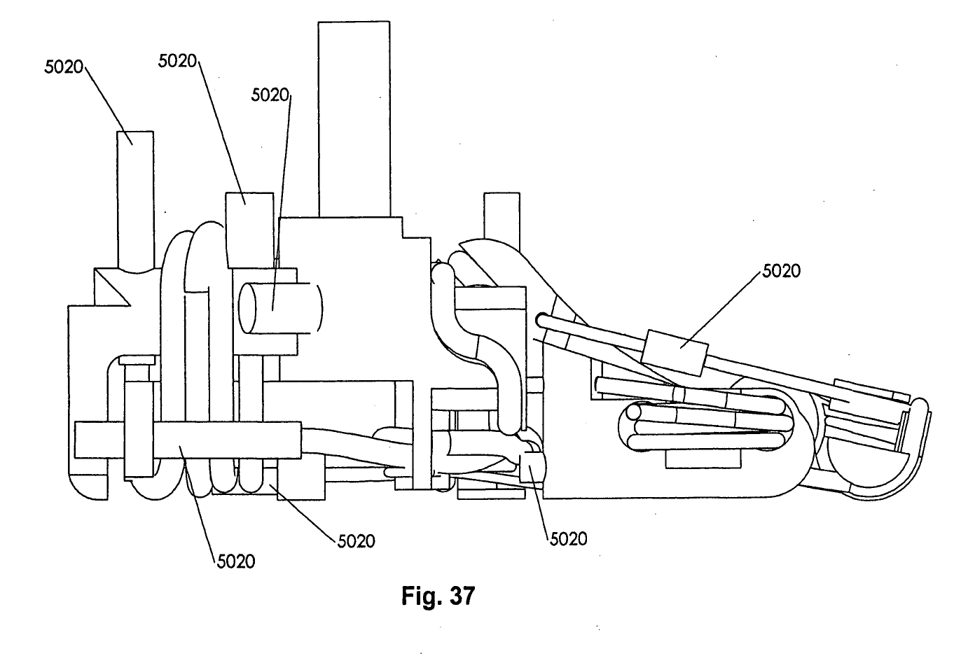

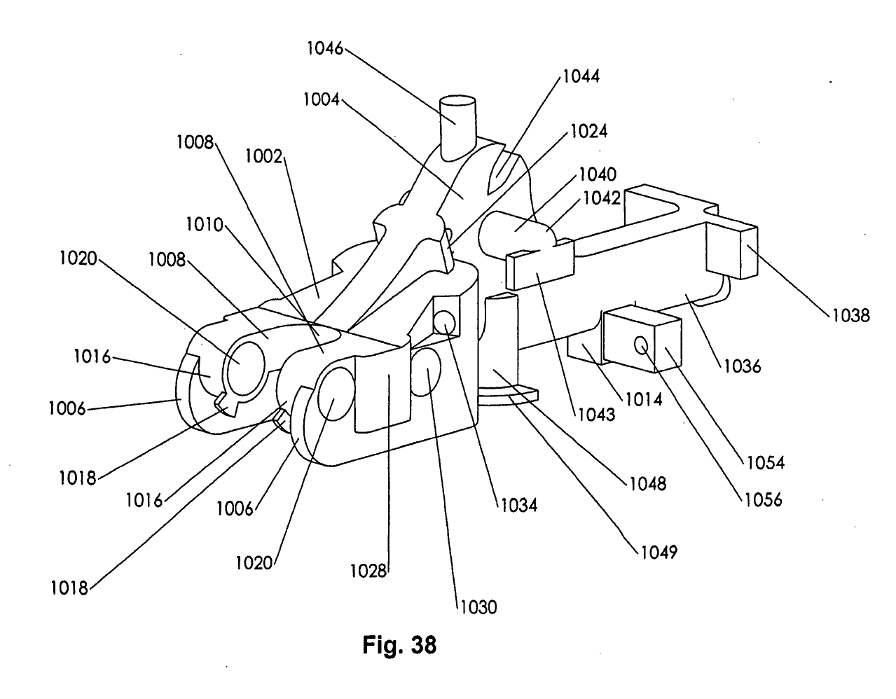

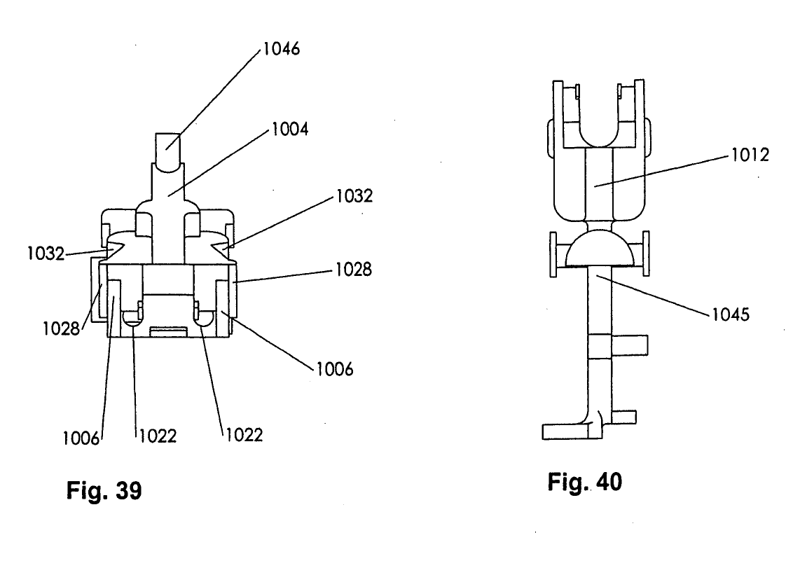

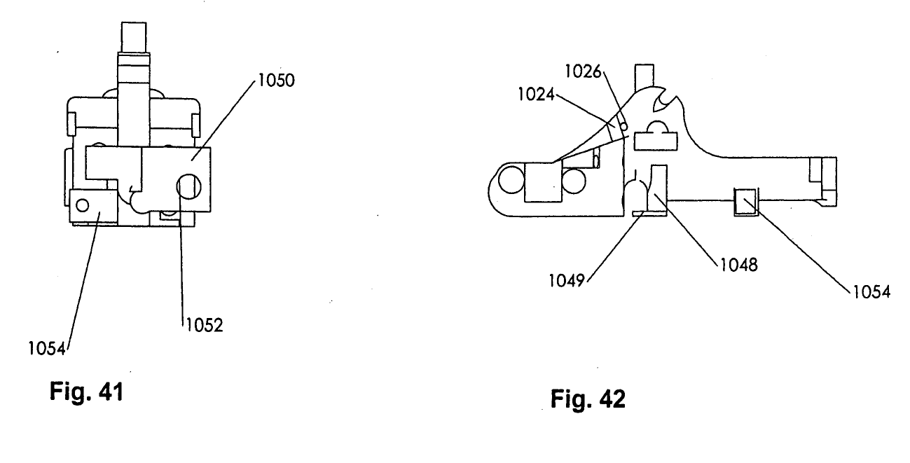

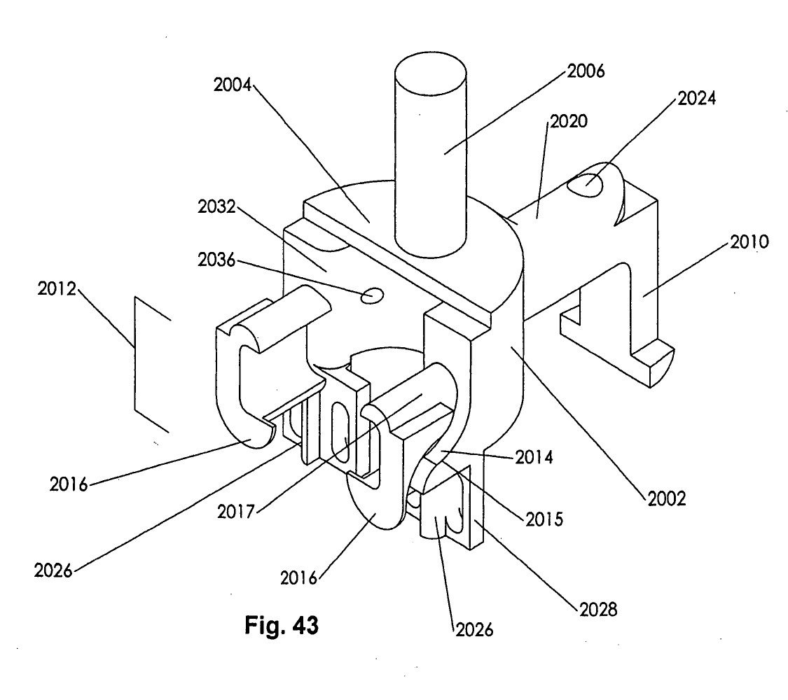

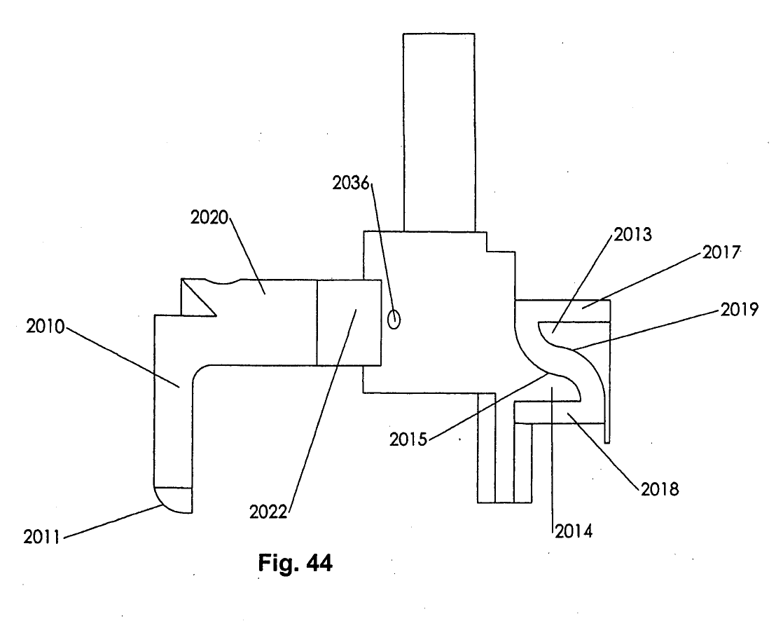

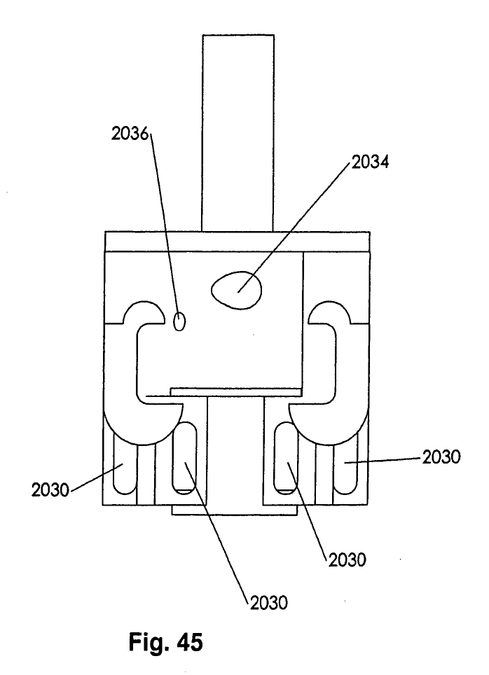

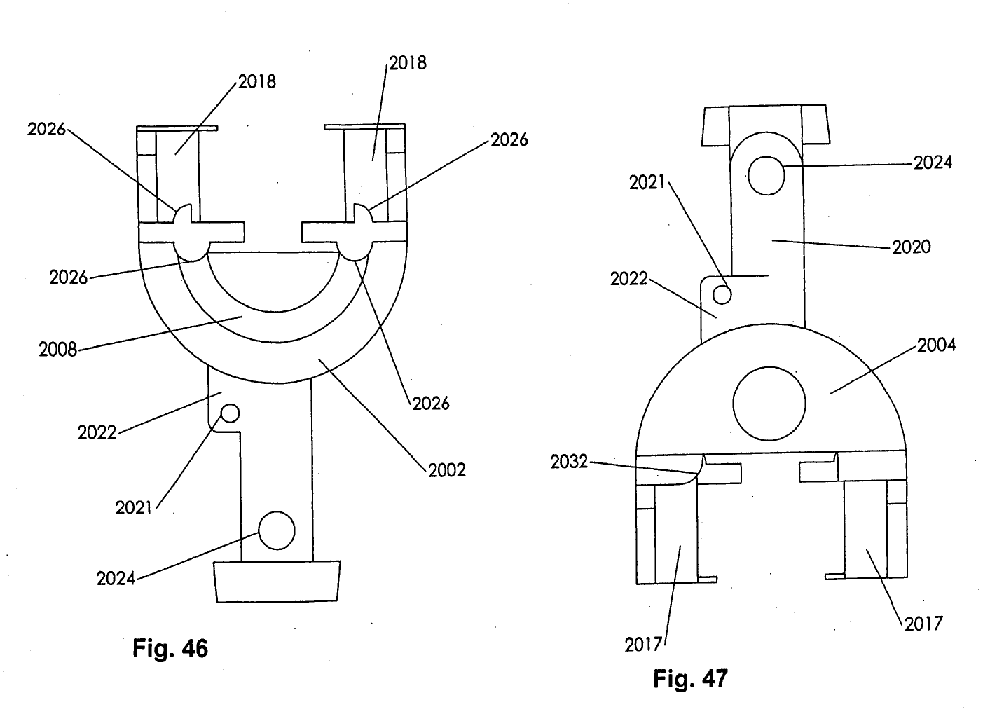

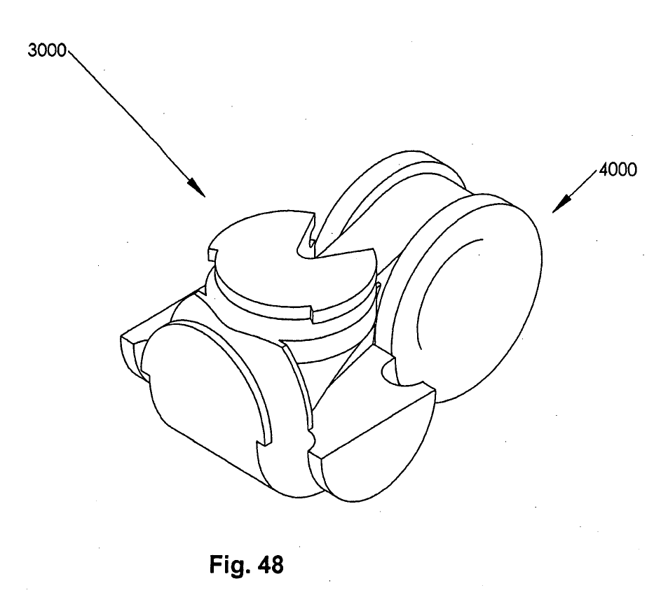

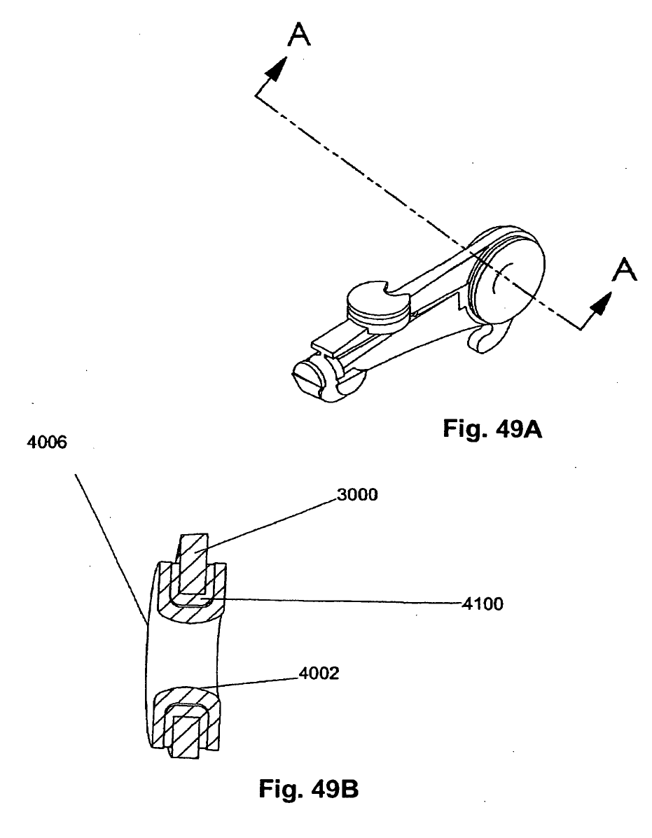

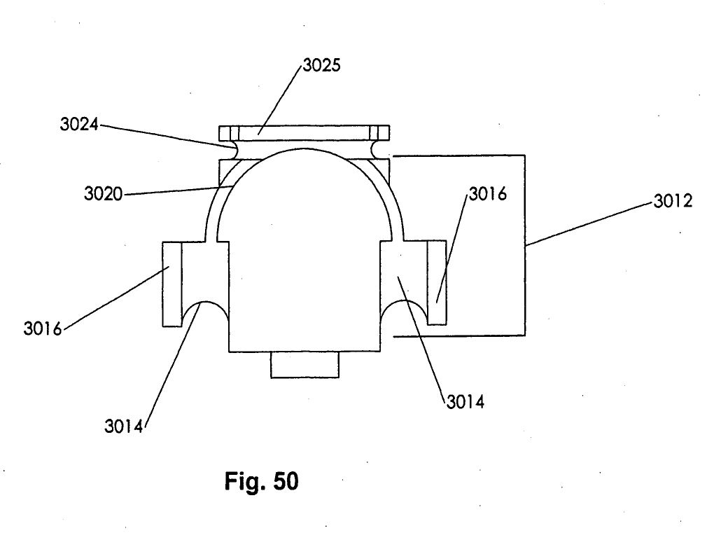

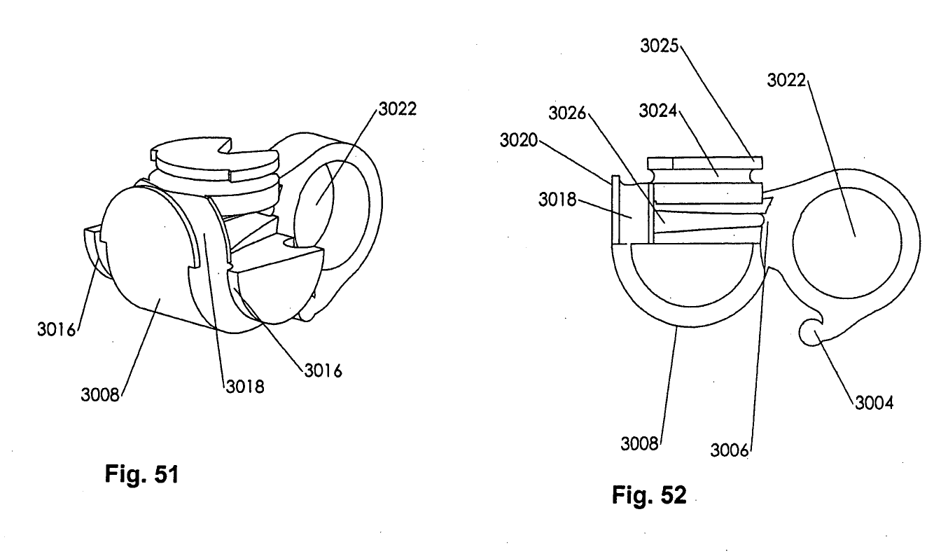

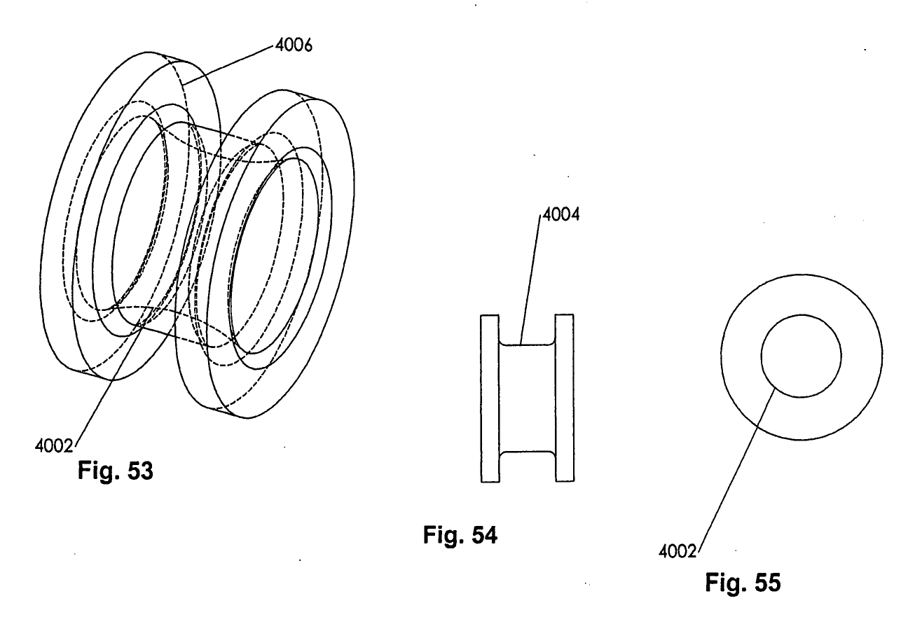

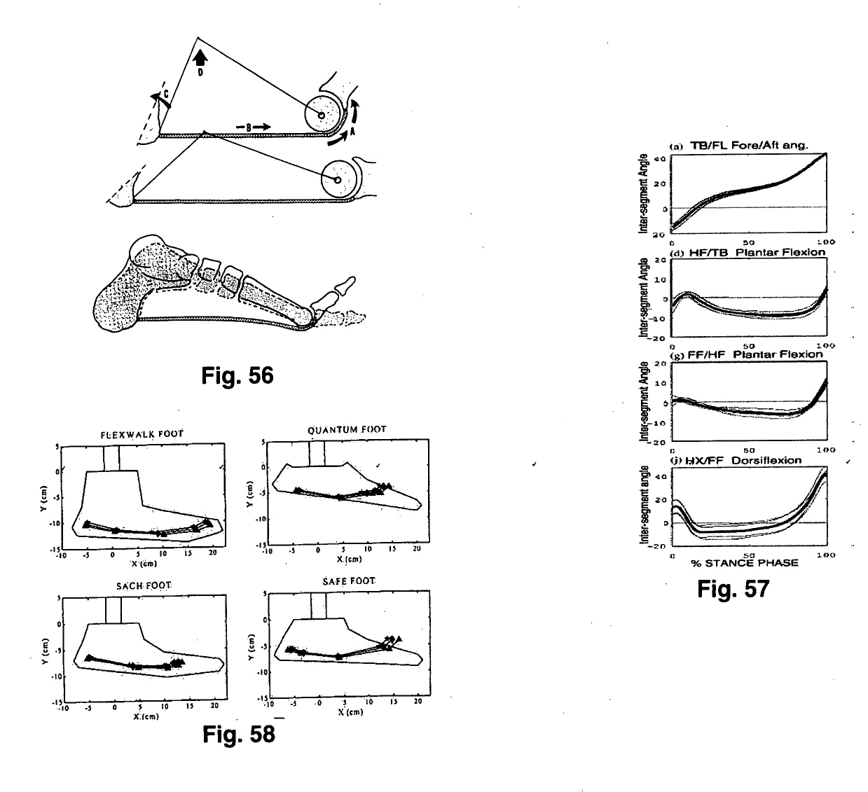

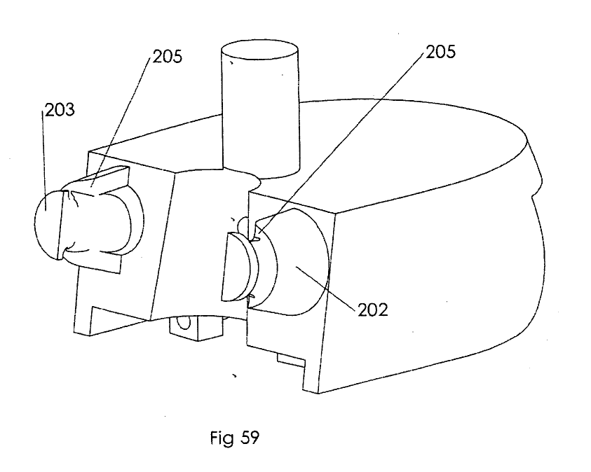

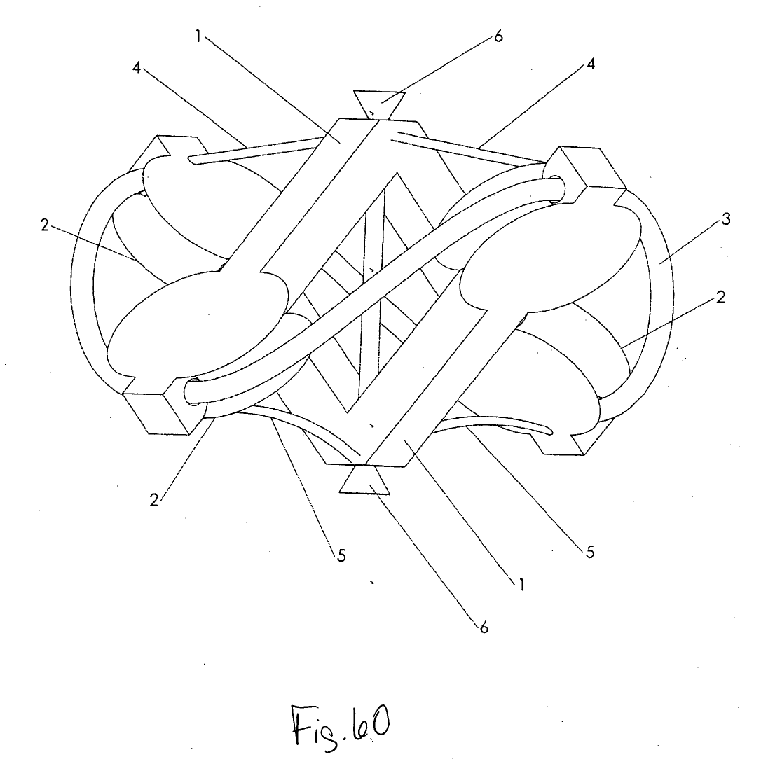

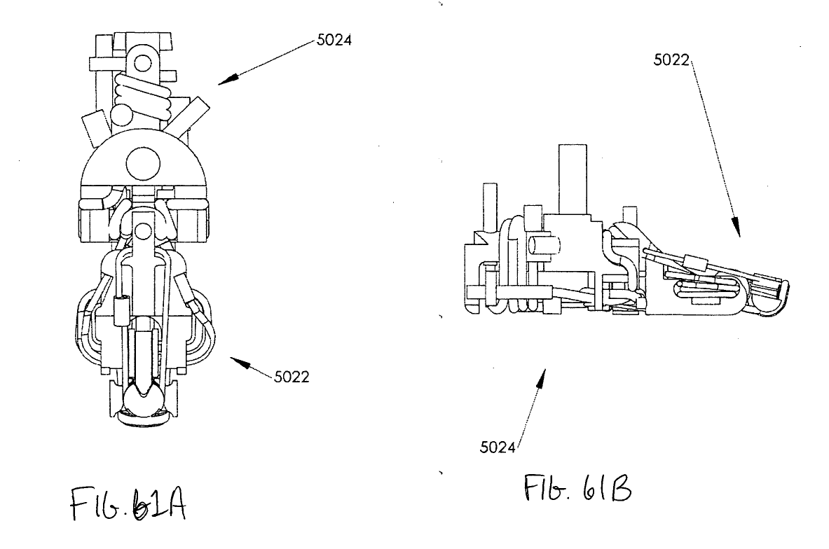

An operation of a limited twist midfoot joint, embodiments of which are shown, for example, in FIGS. 30-55 and 59-61B, may be described in the following. Beginning with the heel strike of the prosthetic foot, the heel strike pad 2011 may compressed and transfers the weight to the heel strike support 2010. This arrangement may keep the force loading within the heel 2000. As the gait progresses, the pylon 2006 may move to an about perpendicular arrangement with the ground. This may be the neutral or flat configuration. In this configuration, weight may be transferred from the pylon 2006, to the forefoot 1000, through the heel 2000. The exact point of weight transfer may be the contact point of the limited twist midfoot twist rope forefoot sheave 1040 and the underside of the limited twist midfoot twist rope superior sheave 2017. The weight may be transferred from the forefoot 1000 to the ground by the walking surface 1006, and the weight stability platform 1014. The weight stability platform 1014 may be situated to be rearward of the pylon 2006, and the walking surface 1006 may be forward of the pylon 2006. This may be an energetically stable configuration.

From this point, the pylon 2006 may advance forward by moving the heel 2000 relative to the forefoot 1000. The point of rotation may be the contact point of the limited twist midfoot twist rope forefoot sheave 1040 and the underside of the limited twist midfoot twist rope superior sheave 2017. This point may be rotationally stabilized by the interaction of the limited twist midfoot twist rope forefoot sheave cap 1043 and the center most face of the limited twist midfoot twist rope heel sheave 2012. As the rotation nears the end of the range of motion, the limited twist midfoot rope 5008, the Achilles rope 5010, and the plantar rope 5006 may be all pulled taut by the interaction of the twist rope complex 5002, the Achilles rope complex 5004, and the plantar rope complex 5000. When these ropes are taut, no further rotational advancement of the pylon 2006, relative to the forefoot 1000 may be possible.

Of course, gait may not stop at this point, and the pylon 2006 may continue to advance, relative to the ground. To address this need, the heel 2000 may experience a vertical translation and a plantar rotation relative to the ground. This may be termed heel rise, and it may be accommodated by the foot rotating about the walking surface 1006.

The tensegrity limited twist midfoot joint 5024 may be restrained from free motion in the plantarflexion direction by the interaction of the limited twist midfoot plantarflexion limit rope 5026, the limited twist midfoot plantarflexion limit rope forefoot sheave 1046, and the pylon 2006, for example, when the limited twist midfoot plantarflexion limit rope 5026, the pylon 2006 and the limited twist midfoot plantarflexion limit rope forefoot sheave 1046 cannot move any further away from each other.

The limited twist midfoot joint may have advantages over other tensegrity midfoot joints. The range of motion may be a better match to the task than the universal joint based on a non-twist midfoot joint. Though similar in range of motion to the twist midfoot joint, the limited twist midfoot joint may be much stronger because of the addition of the plantar rope and the Achilles rope. This may allow for use of smaller rope for the twist rope, which in turn may allow for the use of smaller attachment hardware and smaller sheaves.

As to the advantages of the limited twist midfoot joint over other feet in the art of prosthetics, the midfoot joints that other feet employ are compression based or bending based, where motion of the foot is stopped by use of plastic or rubber bumper, or the foot is allowed to bend until the forces are balanced, respectively. For the compression based joints, the torque/angular deflection response may be derived from the plastic or rubber bumper that is used. This response curve may be wholly unlike the response curve of normal human feet. For the bending based midfoot joints, these may be basically leaf springs made of carbon fiber composites. They may not have any limitations on their ranges of motion. This may not be anatomically correct either. The human foot has the subtalar joint and the chopart joint, both located near the highest point of the arch of the foot. After a certain amount of dorsiflexion, the interactions between the subtalar and chopart joint may make any further dorsiflexion impossible, without damaging the soft tissues of the foot. Also, the torque/angular deflection response of these feet may be based on bending, which is not similar to the response of the human foot, which is based on tension and stretching.

Thus the limited twist midfoot joint may have an appropriate torque/angular deflection response curve, and a clearly defined range of motion, may make it more suitable for all types of activities than its predecessors.

In various embodiments, some examples of a compression member may include one or more of heel 10, forefoot 12, big toe 14, middle toe 16, little toe 18, toe race 380, x-brace 500, upper joint member 504, lower joint member 506, forefoot 1000, heel 2000, toe (wide) 3000, and race 4000.

In various embodiments, some examples of a tension member may include one or more of plantar rope 5006, limited midfoot twist rope 5008, Achilles rope 5010, MTP plantarflexion limit rope 5012, MTP dorsiflexion limit rope 5014, MTP axial rope 5016, cross joint coordination rope 5018, twist rope 52, plantarflexion limit rope 54, dorsiflexion limit rope 62, upper knee universal rope 508, lower knee universal rope 510, upper knee stabilization ropes 512, lower knee stabilization ropes 514, knee tightener rope 528, ankle universal rope 400, ankle stabilizer rope 402, ankle limit rope 414, midfoot joint ropes 22, midfoot tightener rope 32, extension limit ropes 38, flexion limit ropes 40, axial rope 28, universal rope 400, 508, 510, stabilization rope 402, 512, 514, limit rope, and tightener rope 528. One or more of the tensile members (e.g., one or more ropes) may be products manufactured by CABLE MANUFACTURING AND ASSEMBLY COMPANY, INC. of Bolivar, Ohio and/or LIQUID METAL, INC. of Tampa, Fla. In general, tension members may be members that function in tension in a structure and may generally be flexible.

In various embodiments, some examples of actuators referred to herein may include pneumatic muscles, for example, McKibben type pneumatic muscles manufactured by SHADOW ROBOT COMPANY™. Other examples of actuators may include springs (e.g., tension/compression based actuators) and/or coiled actuators (e.g., torsional springs). This may include the use of dashpots in conjunction with springs or other actuators, and other mechanisms for tuning the response of actuators. Further examples of actuators may include at least one balloon disposed inside at least one woven sleeves. In operation, the balloon may expand so as to shorten the woven sleeve along its axis. Conversely, if the sleeve is forcibly lengthened, the balloon may be compressed.

In various embodiments, the actuators referred to herein be powered by recovered energy from heel strike and/or the forced dorsiflexion of the foot during gait. Basically, the forced stretch (or heel strike compression) of one actuator may compress the gas in that actuator, pushing the gas into another actuator located in a different anatomic position. That second actuator may change the response of the foot in gait. For example, a harder heel strike due to faster walking may put more gas in the MTP actuator, making the toes stiffer and thus stronger in their energy return. This may be a “passive” actuator function.

A “passive” actuator may be a spring connected to a limit rope at the terminal hardware (e.g., rope termination hardware 5020). Thus, when the limit rope is pulled taut, it may compress the spring, storing energy, and providing a compliant stop to the joint motion.

An “active” actuator may be controlled by compressed gas, for example, providing the “flexing” of the “muscles” of the prosthetic/orthotic/robotic limb. A control system may be used to move the gases as appropriate.

Actuators may run from compression member to compression member. For example, an MTP actuator may connect to a toe and to a forefoot. A midfoot actuator may run from a forefoot to a heel. A coordinating actuator may run from a toe to a heel, for example, spanning both joints. Also or alternatively, actuators may connect to tension members like limit ropes.

In various embodiments, some examples of a sheave may include one or more of midfoot twist tapered midfoot joint outer sheaves 212, front sheaves 104, tall axial shave 110, short axial sheave 111, plantarflexion limit rope sheave 189, midfoot joint outer sheaves 202, tapered midfoot joint outer sheaves 212, side toe sheave 300, distal toe sheave 302, plantarflexion limit rope toe sheave 330, MTP dorsiflexion forefoot sheaves 1016, MTP MTP plantarflexion limit rope sheave 1024, midfoot Achilles rope forefoot sheave 1036, limited twist midfoot twist rope forefoot sheave 1040, limited twist midfoot twist rope forefoot top sheave 1044, limited twist midfoot twist rope forefoot bottom sheave 1045, limited twist midfoot plantarflexion limit rope forefoot sheave 1046, limited twist midfoot plantar rope forefoot sheave 1048, limited twist midfoot twist rope heel sheave 2012, limited twist midfoot twist rope heel superior guide sheave 2017, limited twist midfoot twist rope heel inferior guide sheave 2018, limited twist midfoot Achilles rope heel sheave 2020, limited twist midfoot plantar rope heel sheave 2026, MTP dorsiflexion limit rope toe sheave 3012, MTP dorsiflexion limit rope side sheave 3014, MTP dorsiflexion limit rope distal sheave 3018, and MTP plantarflexion limit rope toe sheave 3024.

In various embodiments, some examples of a guide may include one or more of plantarflexion limit rope guide 191, dorsiflexion limit rope guides 124, MTP dorsiflexion forefoot sheave guide 1018, midfoot Achilles rope forefoot sheave guide 1038, limited twist midfoot twist rope heel superior guide sheave 2013, limited twist midfoot twist rope heel inferior guide sheave 2014, limited twist midfoot twist rope heel inferior sheave guide 2016, and MTP plantarflexion limit rope toe sheave guide 3025.

In various embodiments, some examples of an attachment mechanism may include one or more of axial rope hole 106, dorsiflexion limit rope holes 116, open dorsiflexion limit rope holes 118, plantarflexion limit rope hols 120, open plantarflexion limit rope holes 122, upper midfoot rope hole 176, lower midfoot rope hole 178, tightener rope hole 182, axial rope screw hole attahment 185, axial rope swage loop attachment hole 186, tightener rope hole 210, ankle limit rope holes 220, axial rope hole 312, heel universal rope holes 406, heel stabilizer rope holes 408, lower leg universal rope holes 410, lower leg stabilizer rope holes 412, lower leg limit rope hopes 426, x-brace universal rope holes 516, x-brace stabilization rope holes 518, upper joint member universal rope holes 520, lower joint member universal rope holes 522, upper joint member stabilization rope holes 524, lower joint member stabilization rope holes 526, x-brace tightener rope hole 530, axial rope forefoot hole 1020, dorsiflexion terminal screw holes 1022, plantarflexion limit rope hole 1026, axial rope return hole 1030, axial rope termainl screw holes 1034, limited twist midfoot plantarflexion rope terminal screw hole 1052, limited twist midfoot plantarflexion rope stop hole 1056, limited twist midfoot achilles rope heel stop hole 2021, limited twist midfoot achilles rope heel screw hole 2024, limited twist midfoot plantarflexion rope heel sheave guide holes 2030, limited twist midfoot twist rope heel terminal screw hole 2034, limited twist midfoot twist rope heel terminal stop hole 2036, and axial rope hole 3022.

Various embodiments of the invention may include an adjustment mechanism. The adjustment mechanism may include a screw and a nut. For example, when a rope's terminal hardware (e.g., rope termination hardware 5020) is a screw, the screw may be attached to portion of the foot (e.g., a compression member) with a nut (e.g., via a hole and/or attachment mechanism). Turning the nut on the screw may adjust the effective length (and/or tension) of the rope.

Various embodiments of the invention may include a coordination member. The coordination member may be a tension or compression member that coordinates the motions of two or more joints (e.g., any joints set forth herein).

An embodiment of the invention may include at least a portion of a prosthetic, orthotic, or robotic device for a skeletal animal comprising at least a portion of an artificial tensegrity joint. Examples of a foot including several artificial tensegrity joints are set forth herein, for example, in various embodiments set forth in FIGS. 11-18B, 27A-55, and 59-61B. In various embodiments, the invention may include one or more of any of the aspects set forth herein.

The artificial tensegrity joint may include at least two compression members (e.g., forefoot 1000 and toe 3000) and at least one tension member (e.g., MTP plantarflexion limit rope 5012). The at least one tension member may connect the at least two compression members. The artificial tensegrity joint may include at least one actuator (e.g., midfoot actuator 34 and/or toe actuator 36) connecting the at least two compression members. The at least one actuator may be powered.

The artificial tensegrity joint may have a range of motion. The at least two compression members may be configured to contact each other in some positions within the range of motion of the artificial tensegrity joint. For example, in the limited twist midfoot joint, the forefoot 1000 and heel 2000 may touch each other on the limited twist midfoot twist rope heel sheave 2012 and the limited twist midfoot twist rope forefoot sheave 1040. In another example, the bottom side of the u-shaped body 2002 of heel 2000 may rest on the superior surface of the Achilles rope forefoot sheave 1036, just forward of the weight stability platform 1014.

The at least two compression members may not be directly connected to each other by the at least one tension member. The artificial tensegrity joint may have a range of motion similar to a range of motion of a corresponding natural joint. The device may be configured to be connected to at least one of a non-human animal or a human.

The artificial tensegrity joint may have a structural strength substantially equal to or greater than a corresponding natural joint. The structural strength may be measured by at least one of a torque/angular deflection response curve and an ultimate compressive strength.

Embodiments of the invention may include at least one of an artificial foot (e.g., artificial foot 7000 and/or artificial foot 8000), an artificial ankle (e.g., artificial ankle 7001 and/or artificial ankle 8001), an artificial knee (e.g., artificial knee 7002 and/or artificial knee 8002), an artificial MTP joint (e.g., tensegrity MTP joint 5022, artificial MTP joint 7003, and/or artificial MTP joint 8003), an artificial midfoot joint (e.g., artificial midfoot joint 7004 and/or artificial midfoot joint 8004), an artificial midfoot twist joint (e.g., artificial midfoot twist joint 7006 and/or artificial midfoot twist joint 8006), an artificial limited twist midfoot joint (e.g., tensegrity limited twist midfoot joint 5024), and an artificial leg (e.g., artificial leg 7005). The artificial tensegrity joint may include at least one of an artificial ankle joint (e.g., artificial ankle 7001 and/or artificial ankle 8001), an artificial knee joint (e.g., artificial knee 7002 and/or artificial knee 8002), an artificial MTP joint (e.g., tensegrity MTP joint 5022, artificial MTP joint 7003, and/or artificial MTP joint 8003), an artificial midfoot joint (e.g., artificial midfoot joint 7004 and/or artificial midfoot joint 8004), an artificial twist midfoot joint (e.g., artificial midfoot twist joint 7006 and/or artificial midfoot twist joint 8006), an artificial limited twist midfoot joint (e.g., tensegrity limited twist midfoot joint 5024), and an artificial universal joint (e.g., artificial universal joint 8002).

Embodiments of the invention may include a compound artificial tensegrity joint (e.g., artificial universal joint 8002). The compound artificial tensegrity joint may be a polycentric joint. The invention may include at least two artificial tensegrity universal joints (e.g., as shown in artificial knee joint 7002 of FIGS. 20 and 21 A-21 B). The at least two artificial tensegrity universal joints may be arranged in a series. The at least two artificial tensegrity universal joints may be stacked on top of each other.

Another embodiment of the invention may include at least a portion of an artificial tensegrity joint. The artificial tensgrity joint may include a first compression member, a second compression member, and at least one tension member including at least one twist tension member (e.g., twist rope 52 and/or limited midfoot twist rope 5008). The at least one twist tension member may connect the first compression member to the second compression member. In various embodiments, the invention may include one or more of any of the aspects set forth herein.

The invention may include at least one limiting tension member (e.g., axial rope 28, extension limit rope 38, flexion limit rope 40, plantarflexion limit rope 54, dorsiflexion limit rope 62, ankle limit rope 414, Achilles limit rope 5010, MTP plantarflexion limit rope 5012, MTP dorsiflexion limit rope 5014, and/or MTP axial rope 5016). The at least one limiting tension member may connect the first compression member to the second compression member. At least one attachment mechanism may connect a tension member to at least one of the first compression member, the second compression member, and the tension member.

The invention may include at least a portion of an artificial tensegrity limited twist midfoot joint (e.g., artificial midfoot joint 7004 and/or artificial midfoot joint 8004). The first compression member may include a forefoot (e.g., forefoot 12 and/or forefoot 1000). The second compression member may include a heel (e.g., heel 10 and/or heel 2000). The twist tension member may include a twist rope (e.g., twist rope 52 and/or limited midfoot twist rope 5008). The limiting tension member may include at least one of a plantarflexion limit rope (e.g., flexion limit rope 40, plantarflexion limit rope 54, and/or MTP plantarflexion limit rope 5012), at least one limited twist midfoot plantar rope (e.g., plantar rope 5006), and an Achilles limit rope (e.g., ankle limit rope 414 and/or Achilles limit rope 5010). The limiting tension member may include at least one of the plantarflexion limit rope and the Achilles limit rope. A plurality of attachment mechanisms may connect at least of the twist rope, the plantarflexion limit rope, and the Achilles limit rope to at least one of the heel, the forefoot, and the attachment mechanism. The heel and the forefoot may be artificial. At least one of the twist rope, the plantarflexion limit rope, and the Achilles limit rope may be configured to be pulled taut substantially simultaneously, for example, during use of the artificial tensegrity joint. The at least a portion of an artificial tensegrity limited twist midfoot joint may be a whole tensegrity limited twist midfoot joint.

The artificial tensegrity joint may include a range of motion. The range of motion of the artificial tensegrity limited twist midfoot joint may be between about a maximum excursion in a plantarflexion direction and about a maximum excursion in a dorsiflexion direction. The range of motion of the artificial tensegrity limited twist midfoot joint may be between at least one of about 0.1 degrees and about 120 degrees, about 0.5 degrees and about 60 degrees, about 1 degree and about 30 degrees, and about 1 degree and about 10 degrees.

At least one midfoot actuator (e.g., midfoot actuator 34) may be connected to at least one of a heel (e.g., heel 10 and/or heel 2000) and a forefoot (e.g., forefoot 12 and/or forefoot 1000). The at least one actuator may be powered. The invention may include an attachment mechanism. The attachment mechanism may connect a heel to a natural or artificial proximal anatomic structure.

The artificial tensegrity limited twist midfoot joint (e.g., artificial midfoot twist joint 7006 and/or artificial midfoot twist joint 8006) may have a structural strength substantially equal or greater than a corresponding natural joint. The structural strength may be measured by at least one of a torque/angular deflection response curve and an ultimate compressive strength.

The invention may include at least one limit rope (e.g., axial rope 28, extension limit rope 38, flexion limit rope 40, plantarflexion limit rope 54, dorsiflexion limit rope 62, ankle limit rope 414, Achilles limit rope 5010, MTP plantarflexion limit rope 5012, MTP dorsiflexion limit rope 5014, and/or MTP axial rope 5016) The at least one limit rope may be prestretched. The artificial tensegrity joint may be configured such that no further joint motion may occur in a direction opposed by the at least one limit rope when the at least one limit rope is in a taut configuration. The at least one limit rope may be elastic. The artificial tensegrity joint may stop compliantly as a result of an engagement of the at least one elastic limit rope. After the engagement of the at least one elastic limit rope, the at least one elastic limit rope may be configured to allow a return of energy to at least one of the compression members as the artificial tensegrity joint substantially returns to a position the artificial tensegrity joint was in prior to the engagement of the at least one elastic limit rope.

The artificial tensegrity joint may be configured to absorb an impact shock upon a rapid motion of at least one of the compression members. An adjustable attachment mechanism may connect the heel (e.g., heel 10 and/or heel 2000) to a natural or an artificial proximal anatomic structure.

The artificial tensegrity limited twist midfoot joint may have a range of motion similar to a corresponding natural human midfoot joint. The artificial tensegrity limited twist midfoot joint may have a range of motion similar to a sum of ranges of motion of a human ankle and a human subtalar joint.

The artificial tensegrity limited twist midfoot joint may have a structural strength substantially equal to or greater than a corresponding natural joint. The structural strength may be measured using at least one of a torque/angular deflection response curve and a ultimate compressive strength.

The invention may include at least one sheave. Examples of sheaves are set forth herein, for example, in the table of parts. The sheave may be configured to protect a tension member from damage caused by bending the tension member at a sharp angle. The sheave may be configured to prevent kinking of a wire rope. The sheave may be include a surface around which the tension member may be bent. The at least one sheave may include two midfoot twist rope heel sheaves (e.g., midfoot twist rope heel sheaves 2012). The twist rope may include a midfoot twist rope (e.g., limited midfoot twist rope 5008). The midfoot twist rope may be disposed around each of the two midfoot twist rope heel sheaves. The midfoot twist rope may be configured to substantially block a rotation of the artificial tensegrity limited twist midfoot joint in a dorsiflexion direction when the midfoot twist rope is in a taut configuration about the two midfoot twist rope heel sheaves.

The midfoot joint rope (e.g., midfoot joint rope 22) may create a midfoot virtual axis of motion. A first midfoot virtual axis may pass through geometrical centers of the two limited twist rope forefoot sheaves (e.g., limited twist rope forefoot sheaves 1040). And one or more of the geometrical centers of the two limited twist rope forefoot sheaves may define a plantarflexion direction of motion and a dorsiflexion direction of motion.

A further embodiment of the invention may include an artificial tensegrity MTP joint (e.g, artificial MTP joint 7003 and/or artificial MTP joint 8003) including a plurality of tension members, a plurality of compression members, and a plurality of attachment mechanisms. The plurality of tension members may include an axial rope (e.g., axial rope 28 and/or MTP axial rope 5016), a dorsiflexion limit rope (e.g., extension limit rope 38, dorsiflexion limit rope 62, and/or MTP dorsiflexion limit rope 5014), and a plantarflexion limit rope (e.g., flexion limit rope 40, plantarflexion limit rope 54, and/or MTP plantarflexion limit rope 5012). The plurality of compression members may include a a toe (e.g., big toe 14, middle toe 16, little toe 18, and/or toe (wide) 3000) and a forefoot (e.g., forefoot 12 and/or forefoot 1000). The axial rope, the dorsiflexion limit rope, and the plantarflexion limit rope may connect the toe to the forefoot. The plurality of attachment mechanisms may connect the axial rope, the dorsiflexion limit rope, and the plantarflexion limit rope to a component. The component may include at least one of a toe, a forefoot, and itself. The toe and the forefoot may be artificial. In various embodiments, the invention may include one or more of any of the aspects set forth herein.

Yet another embodiment of the invention may include at least a portion of an artificial tensegrity joint. The artificial tensegrity joint may include a first compression member, a second compression member, a plurality of tension members, and a plurality of attachment mechanisms. The plurality of tension members may include an axial tension member (e.g., axial rope 28 and/or MTP axial rope 5016) and a limiting tension member (e.g., axial rope 28, extension limit rope 38, flexion limit rope 40, plantarflexion limit rope 54, dorsiflexion limit rope 62, ankle limit rope 414, Achilles limit rope 5010, MTP plantarflexion limit rope 5012, MTP dorsiflexion limit rope 5014, and/or MTP axial rope 5016). The plurality of attachment mechanisms may connect each of the plurality of tension members to a component. The component may include at least one of the first compression member, the second compression member, and the plurality of attachment mechanisms. The plurality of tension members may connect the first compression member to the second compression member. In various embodiments, the invention may include one or more of any of the aspects set forth herein.

The artificial tensegrity joint may include at least one of artificial an tensegrity metatarsophilangeal (MTP) (e.g., tensegrity MTP joint 5022, artificial MTP joint 7003, and/or artificial MTP joint 8003), an artificial tensegrity finger joint, an artificial tensegrity thumb joint, and an artificial tensegrity temporomandibular joint. The artificial tensegrity joint may include at least one of artificial tensegrity metatarsophilangeal (MTP) joints, artificial tensegrity finger joints, artificial tensegrity thumb joints, and artificial tensegrity temporomandibular joints.

The first compression member may be a toe (e.g., big toe 14, middle toe 16, little toe 18, and/or toe (wide) 3000). The second compression member may be a forefoot (e.g., forefoot 12 and/or forefoot 1000). The axial tension member may be an axial rope (e.g., axial rope 28 and/or MTP axial rope 5016). The limiting tension member may include at least one of a dorsiflexion limit rope (e.g., extension limit rope 38, dorsiflexion limit rope 62, and/or MTP dorsiflexion limit rope 5014) and a plantarflexion limit rope (e.g., flexion limit rope 40, plantarflexion limit rope 54, and/or MTP plantarflexion limit rope 5012). The plurality of attachment mechanisms may connect each of the axial rope, the dorsiflexion limit rope, and the plantarflexion limit rope to a component. The component may include at least one of the toe, the forefoot, and the plurality of attachment mechanisms. The toe and the forefoot may be artificial. The plurality of tension members may connect the toe to the forefoot.

The at least a portion of the artificial tensegrity MTP joint may be a whole artificial tensegrity MTP joint.

The at least one limit rope (e.g., axial rope 28, extension limit rope 38, flexion limit rope 40, plantarflexion limit rope 54, dorsiflexion limit rope 62, ankle limit rope 414, Achilles limit rope 5010, MTP plantarflexion limit rope 5012, MTP dorsiflexion limit rope 5014, and/or MTP axial rope 5016) may be elastic. The at least one elastic limit rope may be configured to cause the artificial tensegrity joint to stop compliantly as a result of an engagement of the at least one elastic limit rope. After engagement of the at least one elastic limit rope, the at least one elastic limit rope may allow for a return of energy to one of the plurality of compression members as the artificial tensegrity joint returns to about the same position the artificial tensegrity joint was in prior to the engagement of the at least one elastic limit rope. The artificial tensegrity joint may be configured to absorb an impact shock upon a rapid motion of at least one of the plurality of compression members. The at least one limit rope may be prestretched. The artificial tensegrity joint may be configured such that no further joint motion may occur in a direction opposed by the at least one limit rope when the at least one limit rope is in a taut configuration.

The attachment mechanism for attaching the axial rope to the toe may include an axial rope hole (e.g., axial rope hole 106 and/or axial rope hole 3022) in the toe. The axial rope may be disposed through the axial rope hole of the toe and may define an axis of rotation for the toe.

The artificial tensegrity MTP joint may have a range of motion similar to a corresponding natural human MTP joint. The axial rope may be configured to constrain a vertical translation of the toe relative to the forefoot. The axial rope may be configured to limit a vertical movement of the toe relative to the forefoot. The forefoot may be configured to limit movement of the toe along an axis of the axial rope.

The artificial tensegrity MTP joint may configured to have a range of motion around an axis of the toe. The range of motion may range from about a maximal excursion in a plantarflexion direction to about a maximal excursion in a dorsiflexion direction. The range of motion of the artificial tensegrity MTP joint may be between at least one of about 0.1 degrees and about 340 degrees, about 0.1 degrees and about 170 degrees, about 0.5 degrees and about 60 degrees, about 1 degree and about 30 degrees, and about 1 degree and about 15 degrees.

The invention may include a sheave. Examples of sheaves are set forth herein, for example, in the table of parts. The sheave may protect a tension member from damage caused by bending the tension member at a sharp angle. The sheave may be include a surface around which the tension member may be bent. The rope may include a wire rope. The sheave may prevent a kinking of the wire rope.

The sheave may be configured (e.g, have a shape) such that sides of the sheave may be raised, for example, relative to the portion of the sheave that is shaped like the surface of a cylinder. The raised sheaves may be a guide. Examples of guides are set forth herein, for example, in the table to parts. The raised sheaves may project radially outward from the surface of cylinder. The raised sheaves may be bits of metal. The raised sheaves may be configured to block a motion of the rope in a direction of the axis of the cylinder, which may prevent the tension member from coming off of the end of the sheave. The raised sheaves may be configured to ensure that the tension member do not slip off of edges of the sheave.

The invention may include a rope keeper (e.g., top swage and rope keeper 46 and/or bottom rope keep 48). The rope keeper may be configured to maintain the tension member on a designated pathway on the sheave. The rope keeper may maintain the correct placement of the tension member on the sheave. The designated pathway may include a portion of the surface of the sheave, however, it may not be entirely encompassed by one sheave. The rope may trace a path that loops around the sheaves and/or through the space between the sheaves.

The sheave may include a plantarflexion limit rope toe sheave (e.g., top sheave 330 and/or plantarflexion limit rope toe sheave 3024) attached to the toe. The plantarflexion limit rope (e.g., flexion limit rope 40, plantarflexion limit rope 54, and/or MTP plantarflexion limit rope 5012) may be disposed around the plantarflexion limit rope toe sheave. The plantarflexion limit rope may be configured to substantially block a rotation of the toe in the plantarflexion direction when the plantarflexion limit rope is in a taut configuration about the plantarflexion limit rope toe sheave.

The toe (e.g., big toe 14, middle toe 16, little toe 18, and/or toe (wide) 3000) may include a toe central column (e.g., main column 306). The plantarflexion limit rope toe sheave may include a surface on a side facing away from the forefoot. The surface may have a shape substantially like a first portion of a first curved surface of a first cylinder. The first cylinder may have a first axis substantially perpendicular to the toe central column. The first axis may be substantially orthogonal to a superior surface of the toe. The plantarflexion limit rope toe sheave may protrude from the superior surface of the toe in the first axial direction. The length of the plantarflexion limit rope toe sheave in the direction of the first axis may be between about 1.5 times and about 2 times a diameter of the plantarflexion limit rope.

The toe may include a plantarflexion limit rope guide (e.g., plantarflexion limit rope guide 191 and/or MTP plantarflexion limit rope toe sheave guide 3025). The plantarflexion limit rope guide may include a substantially planar surface intersecting the first axis of the first cylinder of the plantarflexion limit rope toe sheave at the superior face of the plantar limit rope toe sheave. The substantially planar surface may protrude radially outward from the first axis in a direction away from the forefoot. The toe may include a first fillet between an inferior face of the plantarflexion limit rope guide and an adjacent portion of the plantarflexion limit rope toe sheave. The toe may include a second fillet between the superior face of the toe and an adjacent portion of the plantarflexion limit rope toe sheave. Examples of fillets include rope guide fillets, front sheave fillets, bottom fillets, top fillets, front actuator hook fillets, rear actuator hook fillets, inner hook fillets, plantarflexion limit rope toe shave back fillets, and/or limited twist midfoot rope forefoot sheave end fillets 1042.

The sheave may be a dorsiflexion toe sheave (e.g., MTP dorsiflexion limit rope toe sheave 3012). The toe may include a toe central column (e.g., main column 306). The toe may have a long axis. The dorsiflexion toe sheave may include two side toe sheaves side toe sheaves (e.g., side sheaves 300 and/or side sheaves 3014) and a distal toe sheave distal toe sheave (e.g., top sheave 302 and/or distal sheave 3018). Each side toe sheave may be located on a side of the toe central column and may be located proximally to a distal top of the toe.

A most distal to a most inferior surface portion of the side toe sheave may be shaped like a first curved surface portion of a first cylinder. The first cylinder may have a first axis substantially parallel to the axial rope. The distance between the distal tip of the toe and the most distal surface portion of the side toe sheave is about two times the diameter of the dorsiflexion limit rope (e.g., extension limit rope 38, dorsiflexion limit rope 62, and/or MTP dorsiflexion limit rope 5014). The first axis of the first cylinder may be located at about the midline height of the toe. The midline height may be measured from a superior location to an inferior location of the side toe sheave.

The toe may include two side toe sheave rope guides (e.g., side sheave guides 3016). Each side toe sheave rope guide may be attached to each of outer side surface of the side toe sheaves. Each side toe sheave may have a first substantially planar surface that intersects about orthogonally with the first axis of the first cylindrical surface portion of the side toe sheave. The first substantially planar surface may protrude radially outward from the first cylinder axis beyond a distal surface and an inferior surface of each side toe sheave. The toe may include a first fillet between the toe central column of the toe and the first curved cylindrical surface portion of each of the side toe sheaves. The toe may include a second fillet between each side toe sheave rope guide nearer the toe central column and the first curved cylindrical surface portion of the corresponding side toe sheave.

The distal toe sheave may be located at the distal tip of the toe. A superior surface of the distal toe sheave may be shaped like a portion of a second curved surface of a second cylinder. The second cylinder may have a second axis perpendicular to the side toe sheaves and the second axis may be parallel to the long axis of the toe. The distal toe sheave may be positioned such that lines tangential to each lateral-most portions of the second cylindrical surface of the distal sheave 3018 may be substantially parallel to lines tangential to distal-most cylindrical surface portions of the corresponding side toe sheaves. A distance between the substantially parallel tangential lines may be about equal to a diameter of the dorsiflexion limit rope.

The toe may include a distal toe sheave rope guide (e.g., distal sheave guide 3020). The distal toe sheave guide may be attached to the most distal tip of the distal toe sheave (e.g., top sheave 302 and/or distal sheave 3018). The distal toe sheave may have a second substantially planar surface configured to substantially orthogonally intersect the second axis of the second cylindrical surface of the distal toe sheave. The second substantially planar surface may protrude radially outward from a second cylinder axis beyond the superior and lateral surface of the distal toe sheave. The toe may include a first fillet between a proximal face of the distal toe sheave rope guide and an adjoining surface of the distal toe sheave. The toe may include a second fillet between a distal-most face of the main column of the toe and the adjoining surface of the distal toe sheave.

The forefoot toe support sheave may include two toe support rope guides (e.g., limit rope guides 126). The two toe support guides may be on medial and lateral sides of the toe support sheave. Each guide may be disposed next to a different toe (e.g., big toe 14, middle toe 16, little toe 18, and/or toe (wide) 3000). The forefoot (e.g., forefoot 12 and/or forefoot 1000) may include at least two toe supports (e.g., toe supports 102 and/or small foot toe supports 1008). The artificial tensegrity joint may include two side toe sheaves attached to the toe, a front toe sheave (e.g., front sheave 104) attached to the toe, and/or two front toe supprt sheaves (e.g., front sheave 104) each attached to a toe support.

The dorsiflexion limit rope (e.g., extension limit rope 38, dorsiflexion limit rope 62, and/or MTP dorsiflexion limit rope 5014) may be disposed around the distal toe sheave (e.g., top sheave 302 and/or distal sheave 3018) and the two side toe sheaves (e.g., side sheaves 300 and/or side sheaves 3014). The dorsiflexion limit rope may be disposed around each of the toe support sheaves (e.g., front sheave 104). And the dorsiflexion limit rope may be configured to substantially block a rotation of the toe in a dorsiflexion direction when the dorsiflexion limit rope is in a taut configuration about at least one of the distal toe sheave, the side toe sheaves, and the toe support sheaves.

The invention may include an axial toe bearing assembly. The forefoot (e.g., forefoot 12 and/or forefoot 1000) may include at least one toe support (e.g., toe support 102 and/or small foot toe support 1008). The axial toe bearing assembly may include at least one component. The component may include at least one of toe races (e.g., toe races 380 and/or races 4000) with a curved inner surface (e.g., curved inner surface 4002), needle bearings, radial bearings, roller bearings, ball bearings, plastic bearings, liquid metal coatings, and other coatings that have sufficient strength and lubricity. For example, sufficient strength may be defined as having sufficient strength such that something does not break. In another example, sufficient lubricity may be defined as having sufficient lubricity such that something does not bind.

The axial toe bearing assembly may be disposed in a location. The location may be at least one of around the axial rope (e.g., axial rope 28 and/or MTP axial rope 5016), between the toe (e.g., big toe 14, middle toe 16, little toe 18, and/or toe (wide) 3000) and the axial rope, and between the toe and the toe support. The axial toe bearing assembly may be configured to reduce friction between the toe and other parts of the artificial tensegrity joint that the toe may contact during use.

The forefoot (e.g., forefoot 12 and/or forefoot 1000) may include at least two toe supports (e.g., toe support 102 and/or small foot toe support 1008). The toe supports may flank at least one of the toe, toe bearings (e.g., bearings 384 and/or bearings 4100), and a toe race (e.g., toe race 380 and/or race 4000). The toe supports may be configured to block the translation of at least one of the toe, toe bearings, and toe race along the axial rope. The artificial tensegrity joint may have structural strength substantially equal to or greater than a corresponding natural joint. The structural strength may be measured by at least one of a torque/angular deflection response curve and an ultimate compressive strength.

The toe may include a top beam (e.g., top beam 342), a central column (e.g., central column 306), and a thin web (e.g., thin web 340 and/or toe thin web 3026). The top beam, the central column, and the thin web may be configured to respond to a force loading. The force loading may be in the manner of an I beam. The top beam and the central column may be joined by the thin web. Compressive forces applied orthogonally to the top beam or the central column may load the thin web primarily in shear, for example, as opposed to compression. The invention may include an attachment mechanism for connecting the forefoot to a natural or artificial midfoot joint or a natural or artificial heel.

The invention may include at least one toe actuator (e.g., toe actuator 36). The at least one toe actuator may be connected to at least one toe and to the forefoot. The at least one toe actuator may be powered.

The invention may include at least one component. The at least one component may include at least one of bearings (e.g., bearings 384 and/or bearings 4100), races (e.g., toe races 380 and/or races 4000), screw swages (e.g., screw swages 42), stop swages (e.g., stop swages 44), stop swage keeper (e.g., stop swage keeper 46), bottom rope keeper (e.g., bottom rope keeper 48), and rope guide keepers (e.g., rope guides keeper 50). The invention may include an attachment mechanism for connecting the component to the toe (e.g., big toe 14, middle toe 16, little toe 18, and/or toe (wide) 3000) or to the forefoot (e.g., forefoot 12 and/or forefoot 1000).

The invention may include an adjustment mechanism. The adjustment mechanism may be configured to adjust the length of a tension limiting member (e.g., axial rope 28, extension limit rope 38, flexion limit rope 40, plantarflexion limit rope 54, dorsiflexion limit rope 62, ankle limit rope 414, Achilles limit rope 5010, MTP plantarflexion limit rope 5012, MTP dorsiflexion limit rope 5014, and/or MTP axial rope 5016). The tension limiting member may substantially cease motion of the toe in at least one of a planarflexion direction and a dorsiflexion direction, for example, so as limit a range of motion of the toe about the axial rope (e.g., axial rope 28 and/or MTP axial rope 5016) in the at least one of the planarflexion direction and the dorsiflexion direction.

A yet further embodiment of the invention may include a method for ambulating using a prosthetic, orthotic, or robotic foot. The method may include performing a toe off step. In various embodiments, the invention may include one or more of any of the aspects set forth herein.

The foot may include an artificial tensegrity joint. The invention may include bending a prosthetic, orthotic, or robotic artificial tensegrity joint. The joint may include at least one of artificial ankle joints (e.g., artificial ankle 7001 and/or artificial ankle 8001), artificial MTP joint (e.g., artificial MTP joint 7003, artificial MTP joint 8003, and/or tensegrity MTP joint 5022), artificial midfoot joints (e.g., artificial midfoot joint 7004 and/or artificial midfoot joint 8004), artificial twist midfoot joints (e.g., artificial midfoot twist joint 7006 and/or artificial midfoot twist joint 8006), artificial limited twist midfoot joints (e.g., artificial limited twist midfoot joint 8007 and/or tensegrity limit twist midfoot joint 5024), and artificial knee joints (e.g., artificial knee 7002).

The artificial limited twist midfoot joint may include one or more of includes the Achilles rope (e.g., Achilles rope 5010), the plantar rope (e.g., plantar rope 5006), the Achilles rope complex, the plantar rope complex, the limited twist rope (e.g., limited twist midfoot rope 5008) and its sheaves, the forefoot (e.g., forefoot 1000) and the heel (e.g., heel 2000).

The joint may include a first compression member, a second compression member, and a tension member connecting the first and the second compression members. The bending may include at least one of applying force to the first compression member, applying tension to the tension member, and applying a force to the second compression member. The position of the first compression member with respect to the second compression member may be changed by the bending. And applying force to the first compression member may apply tension to the tension member.

Still another embodiment of the invention may include a compound artificial tensegrity joint. The compound artificial tensegrity joint may include at least three compression members and at least two tension members. The first tension member may connect to the first compression member and the second compression member to form a first connected simple artificial tensegrity joint. The second tension member may connect to the second and third compression members to form a second connected simple artificial tensegrity joint. In various embodiments, the invention may include one or more of any of the aspects set forth herein.

The invention may include a fourth compression member and a third tension member. The third tension member may connect to the fourth compression member and to the third compression member to form a third connected simple artificial tensegrity joint. At least two of the first, second, and third connected simple artificial tensegrity joints may be arranged in series. And at least two of the first, second, and third connected simple artificial tensegrity joints may be arranged in parallel.

A still further embodiment of the present invention includes at least a portion of an artificial prosthetic or orthotic leg for a human or robot. The at least a portion of an artificial leg (e.g., artificial leg 7005) may include a knee, an ankle, and an attachment mechanism configured to connect the at least a portion of the artificial leg to a proximal part of the human or the robot. The knee or the ankle may include at least one artificial tensegrity joint. The knee and the ankle may be artificial. In various embodiments, the invention may include one or more of any of the aspects set forth herein.

The invention may include an attachment mechanism configured to connect the artificial leg to a distal structure of the human or the robot. Both the knee and the ankle may each include at least one artificial tensegrity joint. The invention may include an artificial tensegrity midfoot joint (e.g., artificial midfoot joint 7004 and/or artificial midfoot joint 8004). The invention may include an artificial tensegrity MTP joint (e.g., tensegrity MTP joint 5022, artificial MTP joint 7003, and/or artificial MTP joint 8003).

The invention may include a coordination mechanism configured to coordinate the motion of the artificial knee (e.g., artificial knee 7002) and the artificial ankle (e.g., artificial ankle 7001 and/or artificial ankle 8001). The coordination mechanism may include an actuator and the actuator may be configured to cause the artificial ankle and the artificial knee to move substantially synchronously. The coordination mechanism may include a tension member, for example, cross joint coordination rope 5018. The tension member may be configured to cause the artificial ankle and the artificial knee to move substantially synchronously when the tension member is taut. The coordination mechanism may include a compression member. The compression member may be configured to cause the artificial ankle and the artificial knee to move substantially synchronously. The invention may include at least one actuator. The artificial leg may include at least two compression members. The at least one actuator may be to each of the two compression members. And the at least one actuator may be powered. In various embodiments, the coordination mechanism may be a coordination member and/or an actuator that directly creates or alters the motion of more than one joint set forth herein.

Another embodiment of the invention may include at least a portion of an artificial tensegrity universal joint (e.g., artificial universal joint 8002 and/or artificial tensegrity joint 7). The artificial tensegrity universal joint may include a first compression member, a second compression member, and a plurality of tension members. The first and second compression members may include one or more of x-brace 500, upper joint member 504, and lower joint member 506, compression member 1. The plurality of tension members may include a universal rope (e.g., ankle universal rope 400, upper knee universal rope 508, lower knee universal rope 510, and/or universal rope 3) connecting the first compression member to the second compression member and a tightener rope (e.g., knee tightener rope 528 and/or tightener rope 6) connecting the first compression member to the second compression member.

The artificial tensegrity universal joint (e.g., artificial universal joint 8002) may further include at least one tension member attachment mechanism connecting one of the tension members to a component. The component may include at least one of the first compression member, the second compression member, and the at least one tension member attachment mechanism. Each compression member (e.g., x-brace 500, upper joint member 504, and/or lower joint member 506) may include two ends. Each compression member may include a middle. The universal rope (e.g., ankle universal rope 400, upper knee universal rope 508, and/or lower knee universal rope 510) may be configured to provide a multi-axial rotation of the first compression member relative to the second compression member. The tightener rope may be configured to compress the artificial tensegrity universal joint by pulling the first compression member and the second compression member toward each other. The pulling may stress the universal rope (e.g., ankle universal rope 400, upper knee universal rope 508, and/or lower knee universal rope 510). In various embodiments, the invention may include one or more of any of the aspects set forth herein.

The universal rope may substantially contact a first end of the first compression member, a second end of the second compression member, a third end of the first compression member, and a fourth end of the second compression member. The universal rope may substantially contact a first end of the first compression member first, then a second end of the second compression member, then a third end of the first compression member, and then a fourth end of the second compression member.

The connection of the first compression member and the second compression member by the universal rope may create a first virtual axis of motion and a second virtual axis of motion. The first axis may be perpendicular to the face of the first compression member. The first axis may substantially intersect the two locations where the universal rope substantially may contact the first compression member. The second axis may be perpendicular to the face of the second compression member. The second axis may substantially intersect the two locations where the universal rope substantially may contact the second compression member. The first compression member and the second compression members may face each other. The connection of the first compression member and the second compression member by the universal rope may create a first virtual axis of motion and a second virtual axis of motion. The first axis may be perpendicular to the face of the first compression member. The first axis may be more proximal to the second compression member than a first line that substantially intersects the two locations where the universal rope substantially may contact the first compression member. The second axis may be perpendicular to the face of the second compression member. The second axis may be more proximal to the first compression member than a second line that substantially intersects the two locations where the universal rope substantially may contact the second compression member.

A range of motion of the artificial tensegrity universal joint, from a substantially maximal excursion in one direction to a substantially maximal excursion in an opposite direction about a second universal joint axis, may be between at least one of about 0.1 degrees and about 170 degrees, about 0.5 degrees and about 60 degrees, about 1 degree and 30 degrees, and about 1 degree and about 15 degrees. A range of motion of the artificial tensegrity universal joint, from a substantially maximal excursion in one direction to a substantially maximal excursion in an opposite direction about a first universal joint axis, may be between at least one of about 0.1 degrees and about 170 degrees, about 0.5 degrees and about 60 degrees, about 1 degree and about 30 degrees, and about 1 degree and about 15 degrees.

At least one stabilizer rope (e.g., ankle stabilizer rope 302, upper knee stabilization rope 512, lower knee stabilization rope 514, and/or stabilization rope 4) may connect to the first compression member and the second compression member and may be configured to substantially prevent a multi-axial rotation of the joint in some directions. The at least one stabilizer rope may be prestretched. Substantially no further joint motion may be possible in the direction opposed by the stabilizer rope. The stabilizer rope may connect to at least one of the ends of the first compression member at a first connection portion and to substantially the middle portion of the second compression member at a second connection point. The stabilizer rope may substantially block motion of the first connection point and the second connection point away from each other. The at least one stabilizer rope may be elastic and may allow the first compression member and the second compression member to pivot on the universal rope.

The invention may include a rope keeper (e.g., top swage and rope keeper 46 and/or bottom rope keeper 48). The rope keeper may be configured to maintain the tension member on a designated pathway.

The invention may include a sheave. The sheave may be configured to protect a tension member from damage caused by bending at sharp angles. For example, a sharp angle may be defined as a radius of bending which may be less than a radius of a cross section of the tension member. The sheave may be include a surface around which the tension may be bent. The tension member may be a wire rope. The sheave may substantially prevent a kinking of the wire rope.

The invention may include a rope keeper. The rope keeper (e.g., top swage and rope keeper 46 and/or bottom rope keeper 48) may maintain an intended placement of the tension member on the sheave. The sheave may include raised sides. The raised sides may be a guide and may prevent a corresponding rope from slipping off of edges of the sheave.

The invention may include a tightener rope adjustment mechanism. The tightener rope adjustment mechanism (e.g., tightener rope hole 210 and/or x-brace tightener rope hole 530) may be configured to adjust a length of the tightener rope (e.g., knee tightener rope 528). Adjusting the length of the tightener rope may alter a tension of the tightener rope tension, and may thereby alter tension of the universal rope (e.g., ankle universal rope 400, upper knee universal rope 508, and/or lower knee universal rope 510).

At least one limit rope may connect the first compression member to the second compression member, for example, as shown in FIGS. 20 and 21A-21B. The at least one limit rope may be configured to limit a multi-axial rotation of the artificial tensegrity universal joint (e.g., artificial universal joint 8002, artificial tensegrity joint 7). The at least one limit rope 5 may be configured to substantially block further motion of the first compression member relative to the second compression member in a direction of the at least one limit rope, for example, when the at least one limit rope is in a taut configuration.

A length of the limit rope may be configured to be adjustable, for example, so as to alter a range of motion of the artificial tensegrity universal joint. The at least one limit rope may be prestretched; substantially no further joint motion may occurs in a direction opposed by the at least one limit rope when the at least one limit rope is in a taut configuration. The at least one limit rope may substantially contact at least one end of the second compression member and a middle of the first compression member, for example, so as to substantially block a motion of the at least one end of the second compression member and the middle of the first compression member away from each other when the at least one limit rope is in an engaged and/or taut configuration.

The at least one limit rope may be elastic, for example, so as to convey on the artificial tensegrity universal joint at least one of the following characteristics: the artificial tensegrity universal joint may stop compliantly as a result of an engagement (e.g., pulling taut) of the elastic limit rope. After the engagement of the elastic limit rope, the elastic limit rope may may allow for a return of energy to one of the compression members as the joint returns to substantially the same position it was in prior to the engagement of the elastic limit rope. The artificial tensegrity universal joint may be configured to absorb an impact shock upon rapid motion of the compression members. For example, rapid motion may include going from a substantially no load to a substantially peak load in less than 0.25 seconds.

The invention may include an anti-twist mechanism. The anti-twist mechanism may be configured to substantially prevent a twisting of the first compression member relative to the second compression member. The anti-twist mechanism may include an element or a mechanism. The element or the mechanism may include at least one of rope guides, anti-twist tension member attachment mechanisms, a third tension member, geometric arrangements of the first compression member and the second compression member, and geometric interference between the first compression member and the second compression member.

The anti-twist mechanism may have a geometric configuration that imparts “anti-twist” properties. For example, the “U” shaped members (e.g., U-shaped body 2002) of the universal joint may be an anti-twist mechanism. In such an anti-twist mechanism, the vertical part of the “U” shaped members may be very long, for example, so that the “U” shaped members define a rope path, and a rope disposed on the rope path may be at its lowest energy configuration (e.g., stable configuration) at a middle position on the U-shaped member and/or rope path. In another example, the ends of the “U” shaped members may be wider than the middle portion of the U-shaped member, so as to accomplish substantially the same result. In a further example, additional ropes and attachment mechanisms may be used in an anti-twist mechanism, for example, an anti-twist rope.

An external attachment mechanism may connect at least one of the first compression member and the second compression member to a component. The component may includes at least one of a third compression member that is not a portion of the artifical tensegrity universal joint, a tension member that is not a portion of the artifical tensegrity universal joint, and a second joint that is not a portion of the artifical tensegrity universal joint. At least one actuator may be connected to the first compression member and the second compression member. The actuator may be powered.

The artificial joint may include at least one of artificial midfoot joints (e.g., artificial midfoot joint 7004 and/or artificial midfoot joint 8004), artificial ank ankle joints (e.g., artificial ankle 7001 and/or artificial ankle 8001), artificial knee joints (e.g., artificial knee 7002), and artificial spinal joints.

The artificial tensgrity universal joint may be an artificial tensegrity midfoot joint. The universal rope may be a midfoot outer sheaves (e.g., midfoot joint outer sheaves 202, tapered midfoot joint outer sheaves 212). The tightener rope may be a midfoot tightener rope (e.g., midfoot tightener rope 32). The first compression member may be a forefoot (e.g., forefoot 12 and/or forefoot 1000). The second compression member may be a heel (e.g., heel 10 and/or heel 2000). The midfoot joint rope and the midfoot tightener rope may connect the heel to the forefoot in the artificial tensegrity midfoot joint. The at least a portion of the artificial tensegrity midfoot joint 8004 may include a whole tensegrity midfoot joint.

The heel may include two midfoot outer sheaves (e.g., midfoot joint outer sheaves 202, tapered midfoot joint outer sheaves 212). At least one stabilization rope (e.g., ankle stabilizer rope 402, upper knee stabilization rope 512, and/or lower knee stabilization rope 514) may be disposed along the two midfoot outer sheaves of the heel to a forefoot. The stabilization rope may be configured to prevent a side-to-side motion of the forefoot, for example, in a plane including the two midfoot outer sheaves. At least one stabilizer rope may be elastic and may allow the first compression member and the second compression member to pivot on the midfoot tightener rope. At least one stabilizer rope may be prestretched; substantially no further joint motion may be possible in a direction opposed by the stabilizer rope.

The forefoot (e.g., forefoot 12 and/or forefoot 1000) may include a midfoot attachment portion (e.g., midfoot joint attachment 168 and/or midfoot attachment portion 1004). The midfoot attachment portion of the forefoot may include a first distal midfoot protrusion and a second distal midfoot protrusion. Examples of irst and second distal midfoot protrusions include portions of midfoot joint attachment 168 and/or midfoot attachment portion 1004. The two midfoot outer sheaves of the heel may include a third proximal midfoot protrusion and a fourth proximal midfoot protrusion. Examples of third and fourth proximal midfoot protrusions include midfoot joint outer sheaves 202.

The tension member attachment mechanisms may include a first midfoot universal rope hole and second midfoot universal rope hole in the first distal midfoot protrusion and the second distal midfoot protrusion, respectively. Examples of universal rope holes include upper midfoot rope hole 176 and lower midfoot rope hole 178. The third proximal midfoot protrusion and fourth proximal midfoot protrusion may be configured to be wrapped by the tension member. The first universal rope hole, the second universal rope hole, the third proximal midfoot protrusion, and/or the fourth proximal midfoot protrusion may be configured to connect to midfoot joint rope (e.g., midfoot joint rope 22). The midfoot rope may be disposed through the first universal rope hole, the second universal rope hole and may wrap around the the third proximal midfoot protrusion and the fourth proximal midfoot protrusion. The midfoot rope may be configured to substantially prevent a motion of the forefoot and the heel toward each other. The first, the second, the third, and/or the fourth midfoot protrusions may be configured to substantially provide a clearance between the forefoot and the heel.

The forefoot (e.g., forefoot 12 and/or forefoot 1000) and heel (e.g., heel 10 and/or heel 2000) may be configured to pivot on the midfoot rope (e.g., midfoot joint rope 22). The first midfoot protrusion and the second midfoot protrusion may not contact the heel. The third midfoot protrusion and the fourth midfoot protrusion may not substantially contact the forefoot. The first, the second, the third, and/or the fourth midfoot protrusions may be configured to provide a substantially smooth joint motion within a specified range of motion. The midfoot joint rope may create a first midfoot axis of motion and a second midfoot axis of motion. The first midfoot axis may pass through geometric centers of the two midfoot outer sheaves and may define a plantarflexion and a dorsiflexion direction of motion. The second midfoot axis may be substantially perpendicular to the first midfoot axis and may be substantially coaxial with a line connecting a geometric center of the heel and a geometric center of the forefoot. The artificial tensegrity midfoot joints (e.g., artificial midfoot joint 7004 and/or artificial midfoot joint 8004) may have a range of motion substantially similar to a sum of ranges of motion of a human ankle and human subtalar joints. The artificial tensegrity midfoot joint may have a range of motion substantially similar to a corresponding natural human midfoot joint.

A range of motion of the artificial midfoot joint, from a substantially maximal excursion in a plantarflexion direction to a substantially maximal excursion in a dorsiflexion direction, may be between at least one of about 0.1 degrees and about 120 degrees, about 0.5 degrees and about 60 degrees, about 1 degree and about 30 degrees, and about 1 degree and about 10 degrees.

The invention may include at least one limit rope (e.g., extension limit rope 38, flexion limit rope 40, ankle limit rope 414, Achilles limit rope 5010, MTP plantarflexion limit rope 5012, MTP dorsiflexion limit rope 5014, and/or MTP axial rope 5016). The limit rope may connect the forefoot (e.g., forefoot 12 and/or forefoot 1000) and the heel (e.g., heel 10 and/or heel 2000). The limit rope may be configured to substantially limit at least one range of multi-axial rotation of the artificial tensegrity midfoot joint when the limit rope is in a taut configuration. The at least one limit rope may be prestretched; substantially no further joint motion may occur in a direction opposed by the limit rope when the limit rope is in a taut configuration.

The at least one limit rope may be a dorsiflexion limit rope (e.g., extension limit rope 38, dorsiflexion limit rope 62, and/or MTP dorsiflexion limit rope 5014). The dorsiflexion limit rope may be configured to substantially prevent a range of motion of the artificial tensegrity midfoot joint in a dorsiflexion direction. The artificial tensegrity midfoot joint may include a dorsiflexion limit rope sheave (e.g., dorsiflexion limit rope sheave 1016). The forefoot may include a midfoot attachment portion (e.g., midfoot joint attachment 168 and/or midfoot attachment portion 1004). The midfoot attachment portion may include the dorsiflexion limit rope sheave. The artificial tensegrity midfoot joint may include at least one dorsiflexion limit rope sheave guide (e.g., dorsiflexion limit rope sheave guide 1018). The dorsiflexion limit rope guide may flank the dorsiflexion limit rope sheave. The dorsiflexion limit rope may be connected to the heel and may be disposed around the dorsiflexion limit rope sheave and may be guided by the dorsiflexion limit rope guide. The dorsiflexion limit rope may be configured to substantially prevent the rotation of the artificial tensegrity midfoot joint in a dorsiflexion direction when the dorsiflexion limit rope is pulled substantially taut about the dorsiflexion limit rope sheave.

The at least one limit rope may be a plantarflexion limit rope (e.g., flexion limit rope 40, plantarflexion limit rope 54, and/or MTP plantarflexion limit rope 5012). The plantarflexion limit rope may be configured to substantially prevent a range of motion of the artificial tensegrity midfoot joint in a plantarflexion direction. The plantarflexion limit rope sheave (e.g., plantarflexion limit rope sheave 189 and/or MTP plantarflexion limit rope sheave 1024) may include a support sheave and a hook sheave (e.g., plantar flexion sheave surface 192). An example of a support sheave and/or a hook sheave is shown in FIGS. 27A-27C. The support sheave may be a superior surface of a midfoot attachment portion of the forefoot. The support sheave may be proximal to the hook sheave. The support sheave may be shaped like a first curved surface portion of a first cylinder. The first cylinder may have a first axis substantially perpendicular to a long axis of the forefoot and substantially parallel to a plantar surface of the heel.

The at least one plantarflexion limit rope guide (e.g., plantarflexion limit rope guide 191 and/or MTP plantarflexion limit rope toe sheave guide 3025) may include two support sheave rope guides. An example of support sheave rope guides are set forth in FIG. 27B. Each support sheave rope guide may be attached to each outer lateral side surface of the support sheave. Each support sheave rope guide may have a first substantially planar surface that intersects substantially orthogonally with the first axis of the first cylindrical surface portion of the support sheave. The first substantially planar surface may protrude radially outward from the first cylinder axis beyond a distal surface and the superior surface of the support sheave. The artificial tensegrity midfoot joint may include a first fillet and a second fillet. The first fillet and the second fillet may each be located between inside may face of the support sheave rope guides and an adjoining first curved surface of the support sheave. The first fillet and the second fillet may include a smooth surface for contacting the plantarflexion limit rope (e.g., flexion limit rope 40, plantarflexion limit rope 54, and/or MTP plantarflexion limit rope 5012).

The hook sheave (e.g., plantar flexion sheave surface 192) may protrude substantially orthogonally from a first tangent line from the lateral edges of the distal surface of the support sheave. An example of a support sheave is set forth in FIG. 27B. The hook sheave may be shaped like a second curved surface portion of a second cylinder. The second cylinder may have a second axis configured substantially parallel to the long axis of the forefoot and configured substantially parallel to the plantar surface of the heel. The hook sheave may be configured so that a second tangent line and a third tangent line of each lateral-most portion of proximal-most edge of the second cylindrical surface of the hook sheave may be substantially parallel to the first line and may also be tangent to the first cylindrical surface. The second line and the third line may be substantially parallel to each other. A distance between the second line and the third line may be at least substantially equal to a diameter of the plantarflexion limit rope. A distance between inside surface of the support sheave rope guides (e.g., support sheave rope guides as shown in FIG. 27B) may substantially be at least twice a diameter of the plantarflexion limit rope or may be as small as one times the diameter of the plantarflexion limit rope.

The at least one plantarflexion limit rope guide (e.g., plantarflexion limit rope guide 191 and/or MTP plantarflexion limit rope toe sheave guide 3025) may include a hook sheave rope guide (e.g., plantar flexion limit rope sheave 189). The hook sheave rope guide may be attached to the distal surface of the hook sheave. The hook sheave rope guide may have a second substantially planar surface that intersects substantially orthogonally with the axis of the second cylindrical surface portion of the hook sheave. The second substantially planar surface may protrude radially outward from the second cylinder axis beyond the lateral surface and the inferior surface of the hook sheave. The artificial tensegrity midfoot joint may include a third fillet between the inside face of the hook sheave rope guide (e.g., plantar flexion limit rope sheave 189) and the second curved surface of the hook sheave adjacent to the hook sheave rope guide. The artificial tensegrity midfoot joint may include a fourth fillet between the second curved surface of the hook sheave and the midfoot attachment portion of the forefoot adjacent to the second curved surface of the hook sheave. The portion of the midfoot attachment portion of the forefoot adjacent to the fourth fillet and the second curved surface of the hook sheave guides the plantarflexion limit rope. The plantarflexion limit rope (e.g., flexion limit rope 40, plantarflexion limit rope 54, and/or MTP plantarflexion limit rope 5012) may be attached to the heel, may be disposed around the support sheave, may be disposed around the hook sheave, and may be attached to the heel whereby when the plantarflexion limit rope may be pulled taut. The plantarflexion limit rope may be configured to hook the hook sheave and may substantially limit the range of motion of the midfoot joint in the plantarflexion direction.

The artificial midfoot joint (e.g., artificial midfoot joint 7004 and/or artificial midfoot joint 8004) may include a plantarflexion limit rope sheave (e.g., plantarflexion limit rope sheave 189 and/or MTP plantarflexion limit rope sheave 1024). The forefoot (e.g., forefoot 12 and/or forefoot 1000) may include a midfoot attachment portion (e.g., midfoot joint attachment 168 and/or midfoot attachment portion 1004). The midfoot attachment portion of the forefoot may include the plantarflexion limit rope sheave. The plantarflexion limit rope (e.g., flexion limit rope 40, plantarflexion limit rope 54, and/or MTP plantarflexion limit rope 5012) may be disposed around the plantarflexion limit rope sheave. The plantarflexion limit rope sheave may protect the plantarflexion limit rope where it contacts the midfoot attachment portion.

The artificial tensegrity midfoot joint may include at least one plantarflexion limit rope guide (e.g., plantarflexion limit rope guide 191 and/or MTP plantarflexion limit rope toe sheave guide 3025). The plantarflexion limit rope guide may be configured to maintain the plantarflexion limit rope on the designated plantarflexion limit rope pathway. The plantarflexion limit rope may be configured to substantially block the rotation of the artificial tensegrity midfoot joint in the plantarflexion direction when the plantarflexion limit rope is in a taut configuration about the plantarflexion limit rope sheave.

At least one limit rope may be elastic. The joint may stop compliantly as a result of engagement of the elastic limit rope. After engagement of the elastic limit rope, the elastic limit rope may allow for the return of energy to one of the compression members as the joint returns to substantially the same position it was in prior to the engagement of the elastic limit rope. The joint may absorb an impact shock upon rapid motion of the compression members. Rapid motion may include going from a substantially no load to a substantially peak load in less than 0.25 seconds. The invention may include a limit rope sheave and a rope guide.

The forefoot (e.g., forefoot 12 and/or forefoot 1000) may include a midfoot attachment portion (e.g., midfoot joint attachment 168 and/or midfoot attachment portion 1004). One of the limit ropes may be a plantarflexion limit rope (e.g., flexion limit rope 40, plantarflexion limit rope 54, and/or MTP plantarflexion limit rope 5012). The limit rope sheave may be a plantarflexion limit rope sheave (e.g., plantarflexion limit rope sheave 189 and/or MTP plantarflexion limit rope sheave 1024). The rope guide may be a plantarflexion limit rope guide (e.g., plantarflexion limit rope guide 191 and/or MTP plantarflexion limit rope toe sheave guide 3025). The plantarflexion limit rope sheave may protect the plantarflexion limit rope where it may contact the midfoot attachment portion. The plantarflexion limit rope may be connected to the heel (e.g., heel 10 and/or heel 2000). The plantarflexion limit rope may be disposed around the plantarflexion limit rope sheave of the midfoot attachment portion of the forefoot. The limit rope guides may be configured to maintain the plantarflexion limit rope on the designated plantarflexion limit rope pathway. The plantarflexion limit rope may be configured to block the rotation of the artificial midfoot joint in the plantarflexion direction when the plantarflexion limit rope is in a taut configuration about the plantarflexion limit rope sheave of the midfoot attachment portion of the forefoot. The midfoot attachment portion may include the plantarflexion limit rope sheave. The plantarflexion limit rope guides may flank the plantarflexion limit rope sheave. The midfoot attachment portion may include the plantarflexion limit rope guides. The sheave may be positioned to protect the rope where it may contact the midfoot attachment mechanism.

The artificial midfoot joint may include a first plantarflexion limit rope sheave and a second plantarflexion limit rope sheave. The second sheave may be a plantarflexion limit rope attachment mechanism. The first plantarflexion limit rope sheave and the second plantarflexion limit rope sheave may be configured such that the plantarflexion limit rope may be disposed around the first plantarflexion limit rope sheave, may be disposed around and directionally reversed by the second plantarflexion limit rope sheave, and then may be disposed in the reverse direction around the first plantarflexion limit rope sheave.

The invention may include an adjustment mechanism. The adjustment mechanism may be configured to alter the limit of the range of motion of the forefoot (e.g., forefoot 12 and/or forefoot 1000) about the heel (e.g., heel 10 and/or heel 2000) by substantially ceasing motion of the forefoot in one of the dorsiflexion and plantarflexion directions. The adjustment mechanism may be configured to adjust the length of at least one of the limit ropes. The length of the limit ropes may be configured to be adjustable, thereby altering the range of motion of the joint. The midfoot rope (e.g., midfoot joint rope 22) may be configured to limit the motion of the joint. The artificial tensegrity midfoot joint may have similar or better structural strength compared to the corresponding natural joint as measured in terms of the torque/angular deflection response curve or ultimate compressive strength.

The adjustment mechanism may include a midfoot tightener rope hole (e.g., tightener rope hole 182) of the forefoot and a midfoot tightener rope hole (e.g., tightener rope hole 210) of the heel. The midfoot tightener rope (e.g., midfoot tightener rope 32) may be disposed through the midfoot tightener rope hole of the forefoot and the midfoot tightener rope hole of the heel, and may block the motion of the forefoot and the heel away from each other.

The invention may include a tightener rope adjustment mechanism (e.g., tightener rope hole 182 and tightener rope hole 210). The tightener rope adjustment mechanism may be configured to adjust the length of the midfoot tightener rope. Adjusting the length of the midfoot tightener rope may alter a tension on the midfoot tightener rope, thereby altering a tension on the midfoot universal rope (e.g., midfoot joint rope 22 and/or limited midfoot twist rope 5008).

An anti-twist mechanism may be configured to substantially block the twisting motion of the heel (e.g., heel 10 and/or heel 2000) relative to the forefoot (e.g., forefoot 12 and/or forefoot 1000). An attachment mechanism may connect the heel to a natural or artificial proximal anatomic structure. An attachment mechanism may connect the heel to an artificial ankle joint or a natural or artificial leg portion.

At least one midfoot actuator (e.g., midfoot actuator 34) may connect to the heel and to the forefoot. At least one actuator may be powered. At least one component may include one or more of a plantarflexion limit rope (e.g., flexion limit rope 40, plantarflexion limit rope 54, and/or MTP plantarflexion limit rope 5012), a plantarflexion limit rope sheave (e.g., plantarflexion limit rope sheave 189 and/or MTP plantarflexion limit rope sheave 1024), a plantarflexion limit rope guide (e.g., plantarflexion limit rope guide 191 and/or MTP plantarflexion limit rope toe sheave guide 3025), at least one round midfoot outer sheaves (e.g., midfoot joint outer sheaves 202), one or more tapered midfoot joint outer sheaves (e.g., tapered midfoot joint outer sheave), a cushion cavity (e.g, heel cushion cavity 214), a coordination rope attachment (e.g., heel coordination rope attachment 216), and an angled plate mount for sheaves (e.g., angled plate 218). The invention may optionally includes means for attaching each of the components to at least one of the heel and the forefoot.

The joint may be an artificial midfoot twist joint (e.g., artificial midfoot twist joint 7006 and/or artificial midfoot twist joint 8006). The first compression member may be a forefoot (e.g., forefoot 12 and/or forefoot 1000). The second compression member may be a heel (e.g., heel 10 and/or heel 2000). The universal rope may be a twist rope (e.g., twist rope 52 and/or limited midfoot twist rope 5008). The tightener rope may be a midfoot tightener rope (e.g., midfoot tightener rope 32). The twist rope and the midfoot tightener rope connect to the heel and to the forefoot in an artificial tensegrity twist midfoot joint. The midfoot tightener rope may be disposed through the midfoot tightener rope hole (e.g., tightener rope hole 182) of the forefoot and the midfoot tightener rope hole (e.g., tightener rope hole 210) of the heel, blocking the motion of the forefoot and the heel away from each other.

The invention may include a tightener rope adjustment mechanism. The adjustment mechanism may be configured to adjust the length of the midfoot tightener rope. Adjusting the length of the midfoot tightener rope may alter a tension on the midfoot tightener rope, thereby altering a tension on the midfoot universal rope.

At least one midfoot actuator (e.g., midfoot actuator 34) may connect to at least of the forefoot, the heel, and a portion of the heel. At least one actuator may be powered. The twist rope (e.g., twist rope 52 and/or limited midfoot twist rope 5008) may be twisted on either side of the midfoot attachment portion (e.g., midfoot joint attachment 168 and/or midfoot attachment portion 1004) of the forefoot, with the two twists being in mirror image directions, so that a rotation of the forefoot in the dorsiflexion direction may increase the twist, such that the increasing twist acts as a limitation of motion in the dorsiflexion direction. The twist rope may be configured such that the twist rope may be twisted substantially itself on both sides of the midfoot attachment in a manner such that the helical twist of the twist rope twists further when the forefoot may be rotated in the dorsiflexion direction, restricting and blocking further rotation in the dorsiflexion direction.

At least one stabilizer rope may connect to the forefoot (e.g., forefoot 12 and/or forefoot 1000) and to the heel (e.g., heel 10 and/or heel 2000). The stabilizer rope may be configured to block the side to side motion of the forefoot, in the plane of the midfoot outer sheaves (e.g., midfoot joint outer sheaves 202, tapered midfoot joint outer sheaves 212). At least one stabilizer rope may be elastic and may allow the forefoot and heel to pivot on the twist rope (e.g., twist rope 52 and/or limited midfoot twist rope 5008). The midfoot twist may include two outer twist rope heel sheaves (e.g., limited twist midfoot twist rope heel sheave 2012).

The forefoot may have a midfoot attachment portion (e.g., midfoot joint attachment 168 and/or midfoot attachment portion 1004). The midfoot attachment portion may include a proximal portion and a distal portion. The midfoot twist joint may include helical twist rope forefoot sheaves (e.g., limited twist midfoot twist rope forefoot sheave 1040).

The tension member attachment mechanisms may include a first forefoot universal rope hole sheave (e.g., midfoot attachment hole upper 187 and/or midfoot attachment hole lower 188) and a second forefoot universal rope hole sheave (e.g., midfoot attachment hole upper 187 and/or midfoot attachment hole lower 188) in the midfoot attachment portion of the forefoot. The outer twist rope heel sheaves (e.g., limited twist midfoot twist rope heel sheave 2012) may be configured to be wrapped by the midfoot twist rope (e.g., twist rope 52 and/or limited midfoot twist rope 5008).

The first foot universal rope hole sheave and the second forefoot universal rope hole sheave and the helical twist rope forefoot sheaves may connect the forefoot to the midfoot twist rope. The midfoot twist rope may be disposed through the first forefoot universal rope hole sheave (e.g., midfoot attachment hole upper 187 and/or midfoot attachment hole lower 188) and the second forefoot universal rope hole sheave (e.g., midfoot attachment hole upper 187 and/or midfoot attachment hole lower 188). The midfoot twist rope may be configured to twist helically about each of the helical twist rope forefoot sheaves. The midfoot twist rope may wrap around the helical twist rope forefoot sheaves. The midfoot twist rope may be configured to substantially block the motion of the forefoot and the heel toward each other. The helical twist rope forefoot sheaves and the outer twist rope heel sheaves may be configured to provide substantial clearance between the forefoot and the heel. The outer twist rope heel sheaves may be configured to provide substantial clearance between the helical twist rope forefoot sheaves.

The forefoot (e.g., forefoot 12 and/or forefoot 1000) and heel (e.g., heel 10 and/or heel 2000) may be able to pivot on the midfoot twist rope (e.g., twist rope 52 and/or limited midfoot twist rope 5008) while the helical twist rope forefoot sheaves (e.g., limited twist midfoot twist rope forefoot sheave 1040) do not substantially contact the heel and while the outer twist rope heel sheaves (e.g., limited twist midfoot twist rope heel sheave 2012) do not substantially contact the forefoot, thereby providing smooth joint motion within the specified range of motion. The midfoot twist rope may be configured in substantially helical twists around the helical twist rope forefoot sheaves. The midfoot helical twist rope sheaves protect the midfoot twist rope from damage by providing a rigid surface that the midfoot twist rope may bend and wrap around, each helical twist rope forefoot sheave may be configured to limit the range of motion in the dorsiflexion direction of the artificial tensegrity midfoot twist joint (e.g., artificial midfoot twist joint 7006 and/or artificial midfoot twist joint 8006).

In another example, the heel (e.g., heel 10 and/or heel 2000), the forefoot (e.g., forefoot 12 and/or forefoot 1000), and/or other compression members may rest together (e.g., contact each other) in a “neutral” configuration of the limited twist midfoot joint (e.g., tensegrity limited twist midfoot joint 5024). The heel, the forefoot, and/or other compression members may contact each other in one or more locations.

The midfoot joint rope (e.g., midfoot joint rope 22) may create a first midfoot axis of motion and a second midfoot axis of motion. The first midfoot axis may pass through geometrical centers of the two midfoot outer sheaves (e.g., midfoot joint outer sheaves 202, tapered midfoot joint outer sheaves 212), which may define the plantarflexion and dorsiflexion directions of motion. The second midfoot axis may be substantially perpendicular to the first midfoot axis, and may be coaxial with a line that may connect the geometric center of the heel and the geometric center of the forefoot.

The artificial tensegrity midfoot joints (e.g., artificial midfoot joint 7004 and/or artificial midfoot joint 8004) may have a range of motion similar to a corresponding natural human midfoot joint. The range of motion of the artificial midfoot joint, from a substantially maximal excursion in the plantarflexion direction to a substantially maximal excursion in the dorsiflexion direction, may be between at least one of about 0.1 degrees and about 120 degrees, about 0.5 degrees and about 60 degrees, about 1 degree and about 30 degrees, and about 1 degree and about 10 degrees. The artificial tensegrity midfoot joint may have a range of motion similar to a sum of the ranges of motion of the human ankle and human subtalar joints; each midfoot helical twist rope sheave may be at substantially the same height relative to the plantar surface of the heel as the outer twist rope heel sheaves (e.g., limited twist midfoot twist rope heel sheave 2012).

Each helical twist rope forefoot sheave (e.g., limited twist midfoot twist rope forefoot sheave 1040) may be shaped like a first cylinder. The first cylinder may have a first axis configured substantially perpendicular to the long axis of the forefoot and substantially parallel to the plantar surface of the heel. The first axis may be substantially colinear with a line between the outer twist rope heel sheaves. The helical twist rope forefoot sheaves may extend outward from each side of the midfoot attachment portion (e.g., midfoot joint attachment 168 and/or midfoot attachment portion 1004) of the forefoot. The helical twist rope forefoot sheaves may not be so long as to interfere with the outer twist rope heel sheaves (e.g., limited twist midfoot twist rope heel sheave 2012) when the artificial tensegrity twist midfoot joint may pass through a selected artificial tensegrity twist midfoot joint range of motion. The first cylinder may have a first curved surface.

The midfoot twist joint may include two helical twist rope forefoot sheave rope guides (e.g., limited twist midfoot twist rope forefoot sheaves 1040, limited twist midfoot twist rope forefoot top sheave 1044, and/or limited twist midfoot twist rope forefoot bottom sheave 1045). Each helical twist rope forefoot sheave rope guide may be attached to each of the side outer surface of the helical twist rope forefoot sheaves. Each helical twist rope forefoot sheave rope guide may have a first substantially planar surface that intersects substantially orthogonally with the first axis of the substantially flat cylindrical surface of each helical twist rope forefoot sheave (e.g., limited twist midfoot twist rope forefoot sheave 1040). The first substantially planar surface may protrude radially outward from the first cylinder axis beyond the first curved cylindrical surface of the helical twist rope forefoot sheave.

Each forefoot may include a helical twist forefoot sheave rope guide sheave. The helical twist forefoot sheave rope guide sheave may be a notch in the helical twist forefoot sheave rope guide. The notch may have a curved surface so that a rope passing over or through the notch may not be damaged. The first forefoot universal rope hole sheave may be at a height above the helical rope twist forefoot sheaves relative to the heel plantar surface. The first universal rope hole sheave may be a first void within the midfoot attachment portion of the forefoot. The void may have two first openings on the outer sides of the midfoot attachment portion of the forefoot.

The inferior surface of the first forefoot universal rope hole sheave may be shaped like a second curved surface portion of a second cylinder. The second cylinder may have a second axis configured substantially parallel to the plantar surface of the forefoot and parallel to the long axis of the forefoot. The distal and proximal walls of the first void function as first universal rope hole sheave rope guides. The twist midfoot joint may include a first fillet and a second fillet. Each between the proximal and distal sides of the first void and the second curved surface of the first forefoot universal rope hole sheave. The first openings may be at a first distance above a line between the outer twist rope heel sheaves (e.g., limited twist midfoot twist rope heel sheave 2012 and/or midfoot joint outer sheaves 202). The second forefoot universal rope hole sheave may be at a height below the helical twist rope forefoot sheaves (e.g., limited twist midfoot twist rope forefoot sheave 1040 and/or midfoot joint outer sheaves 202) relative to the plantar surface of the heel.

The second midfoot attachment forefoot sheave (e.g., a portion of the artificial midfoot twist joint 7006 and/or tensegrity limited twist midfoot joint 5024) may be a second void within the midfoot attachment portion (e.g., midfoot joint attachment 168 and/or midfoot attachment portion 1004) of the forefoot (e.g., forefoot 12 and/or forefoot 1000). The second void may have two second openings in the outer side surface of the midfoot attachment portion of the forefoot. The second openings may be aligned along the proximal/distal axis below the first openings of the first void. The second openings may be at a second distance below a line between the outer twist rope heel sheaves. The first distance may be substantially equal to the second distance.

The superior surface of the second forefoot universal rope sheave may be shaped like a third curved surface portion of a third cylinder. The third cylinder may have a third axis configured substantially parallel to the plantar surface of the forefoot. The third axis may be configured to be parallel to the long axis of the forefoot. The proximal and distal walls of the void may function as second forefoot universal rope sheave rope guides. And the twist midfoot joint may include a third fillet and a fourth fillet, each between the proximal and distal sides of the second void and the third curved surface of the second forefoot universal rope hole sheave.

Each outer twist rope heel sheave (e.g., limited twist midfoot twist rope heel sheave 2012) may extend distally from the heel (e.g., heel 10 and/or heel 2000) on either outer side of the midfoot attachment portion (e.g., midfoot joint attachment 168 and/or midfoot attachment portion 1004) of the forefoot (e.g., forefoot 12 and/or forefoot 1000). Each outer twist rope heel sheave may be substantially the shape of at least a portion of a cone. The cone may have a proximal diameter and a distal diameter. The proximal diameter may be larger than the distal diameter. The cone may have a fourth axis configured substantially parallel to the plantar surface of the heel and substantially parallel to the long axis of the forefoot when in a flat configuration with the heel. Each outer twist rope heel sheave may be configured to allow clearance for the helical twist rope forefoot sheaves (e.g., limited twist midfoot twist rope forefoot sheave 1040) such that the outer twist rope heel sheaves and the helical twist rope forefoot sheaves do not interfere with each other when the artificial tensegrity twist midfoot joint may pass through a selected artificial tensegrity twist midfoot joint range of motion. The twist midfoot joint may include two outer twist rope heel sheave rope guides (e.g., limited twist midfoot twist rope heel superior guide sheave 2013, limited twist midfoot twist rope inferior guide sheave 2014, and/or limited twist midfoot twist rope heel inferior sheave guide 2016). Each outer twist rope heel sheave rope guide may protrude radially outward, relative to the fourth axis, from the fourth curved conical surface of each outer twist rope heel sheave. Each outer twist rope heel sheave rope guide may be proximal to the midfoot twist rope (e.g., twist rope 52 and/or limited midfoot twist rope 5008). The twist midfoot joint may include fifth and six fillets between the fourth curved conical surface of each outer twist rope heel sheaves distal to the outer twist rope heel sheave rope guides and the distal surface of the outer twist rope heel sheave rope guides.

The invention may include an attachment mechanism that may connect the heel (e.g., heel 10 and/or heel 2000) to an artificial ankle joint (e.g., artificial ankle 7001 and/or artificial ankle 8001) or a natural or artificial leg portion. The invention may include an attachment mechanism that may connect the forefoot (e.g., forefoot 12 and/or forefoot 1000) to the artificial MTP joint (e.g, artificial MTP joint 7003 and/or artificial MTP joint 8003) or toes (e.g., big toe 14, middle toe 16, little toe 18, and/or toe (wide) 3000).

The invention may include at least one limit rope which may be elastic. The joint may stop compliantly as a result of engagement of the elastic limit rope. After engagement of the elastic limit rope, the elastic limit rope may allow for the return of energy to one of the compression members as the joint returns to substantially the same position it was in prior to the engagement of the elastic limit rope. The joint may absorb an impact shock upon rapid motion of the compression members. Rapid motion may include going from a substantially no load to a substantially peak load in less than 0.25 seconds.

The invention may include a plantarflexion limit rope (e.g., flexion limit rope 40, plantarflexion limit rope 54, and/or MTP plantarflexion limit rope 5012). The plantarflexion limit rope may be connected to the heel (e.g., heel 10 and/or heel 2000). The plantarflexion limit rope may be disposed around the plantarflexion limit rope sheave (e.g., plantarflexion limit rope sheave 189 and/or MTP plantarflexion limit rope sheave 1024), located on the midfoot attachment mechanism (e.g., midfoot joint attachment 168 and/or midfoot attachment portion 1004) of the forefoot (e.g., forefoot 12 and/or forefoot 1000). The plantarflexion limit rope may be configured to block the motion of the artificial twist midfoot joint (e.g., artificial midfoot joint 7004 and/or artificial midfoot joint 8004) in the plantarflexion direction when pulled taut.

At least one of the limit ropes may be prestretched, and substantially no further joint motion may occur in the direction opposed by the limit rope when the limit rope is in a taut configuration. An attachment mechanism may connect the heel to a natural or artificial proximal anatomic structure. The artificial twist midfoot joint may have similar or better structural strength compared to the corresponding natural joint as measured in terms of the torque/response curve or ultimate compressive strength.

The invention may include a one or more of each of a twist rope (e.g., twist rope 52 and/or limited midfoot twist rope 5008), a plantarflexion limit rope (e.g., flexion limit rope 40, plantarflexion limit rope 54, and/or MTP plantarflexion limit rope 5012), a plantarflexion limit rope sheave (e.g., plantarflexion limit rope sheave 189 and/or MTP plantarflexion limit rope sheave 1024), a plantarflexion limit rope guide (e.g., plantarflexion limit rope guide 191 and/or MTP plantarflexion limit rope toe sheave guide 3025), at least one round midfoot outer sheaves (e.g., midfoot joint outer sheaves 202), one or more tapered midfoot joint outer sheaves (e.g., tapered midfoot joint outer sheave), a cushion cavity (e.g, heel cushion cavity 214), a coordination rope attachment (e.g., heel coordination rope attachment 216), and an angled plate mount for sheaves (e.g., angled plate 218).

The portion of the artificial tensegrity twist midfoot joint (e.g., artificial midfoot twist joint 7006 and/or artificial midfoot twist joint 8006) may be a whole tensegrity twist midfoot joint. The joint may be an artificial tensegrity midfoot joint. The first compression member may be a forefoot (e.g., forefoot 12 and/or forefoot 1000). The second compression member may be a heel (e.g., heel 10 and/or heel 2000). The universal rope may be a midfoot universal rope (e.g., midfoot joint rope 22 and/or limited midfoot twist rope 5008). The tightener rope may be a midfoot tightener rope (e.g., midfoot tightener rope 32).

The midfoot joint may include two outer rope heel sheaves (e.g., limited twist midfoot twist rope heel sheave 2012). The forefoot may have a midfoot attachment portion (e.g., midfoot joint attachment 168 and/or midfoot attachment portion 1004). The tension member attachment mechanisms may include first and second forefoot universal rope hole sheave (e.g., midfoot attachment hole upper 187 and/or midfoot attachment hole lower 188, limited twist midfoot twist rope forefoot top sheave 1044, and/or limited twist midfoot twist rope forefoot bottom sheave 1045) in the midfoot portion of the forefoot.

The outer rope heel sheaves may be configured to be wrapped by the midfoot universal rope. The first foot universal rope hole sheave and the second forefoot universal rope hole sheave may connect the forefoot to the midfoot universal rope. The midfoot universal rope may be disposed through the first and second forefoot universal rope hole sheaves (e.g., midfoot attachment hole upper 187 and/or midfoot attachment hole lower 188). The midfoot universal rope (e.g., midfoot joint rope 22 and/or limited midfoot twist rope 5008) may be configured to wrap around each of the outer rope heel sheaves. The outer rope heel sheaves and the first foot universal rope hole sheave and the second forefoot universal rope hole sheave may be configured to substantially block the motion of the forefoot and the heel toward each other. The outer rope heel sheaves and the first foot universal rope hole sheave and the second forefoot universal rope hole sheave may be configured to provide substantial clearance between the forefoot and the heel. The forefoot and heel may be able to pivot on the outer rope heel sheaves while the forefoot may not substantially contact the heel thereby providing smooth joint motion within the specified range of motion. The outer rope heel sheaves may protect the midfoot universal rope (e.g., midfoot joint rope 22 and/or limited midfoot twist rope 5008) from damage by providing a rigid surface that the midfoot universal rope may bend and wrap around. Each outer rope heel sheave may be configured to limit the range of motion in the dorsiflexion direction of the artificial tensegrity midfoot joint.

Each outer rope heel sheaves (e.g., limited twist midfoot twist rope heel sheave 2012) may extend distally from the heel on either side of the midfoot attachment region of the forefoot. Each outer rope heel sheave may be substantially the shape of at least a curved surface portion of a cylinder. The portion of a cylinder may have a proximal diameter and a distal diameter. The proximal diameter may be larger than the distal diameter the configuration of the universal rope (e.g., midfoot joint rope 22 and/or limited midfoot twist rope 5008) on the outer rope sheaves substantially blocks the forefoot (e.g., forefoot 12 and/or forefoot 1000) from directly contacting the heel (e.g., heel 10 and/or heel 2000). The cylinder may have a first axis configured substantially parallel to the plantar surface of the heel and substantially parallel to the long axis of the forefoot when in a flat configuration with the heel. The outer rope heel sheaves may be configured to not contact the forefoot directly. The superior and inferior surface of the outer rope heel sheaves may be configured to be below and above the superior and inferior surface of the proximal portion of the midfoot attachment portion (e.g., midfoot joint attachment 168 and/or midfoot attachment portion 1004) of the forefoot respectively. The curved surface of the portion of the cylinder may be on the outer, inferior, and superior surface of the outer rope heel sheaves. The width of the outer rope heel sheaves from proximal to distal surface may be substantially or greater than the cross-sectional diameter of the midfoot universal rope.

The first forefoot universal rope hole sheaves (e.g., midfoot attachment hole upper 187, midfoot attachment hole lower 188, limited twist midfoot twist rope forefoot top sheave 1044, and/or limited twist midfoot twist rope forefoot bottom sheave 1045) may be at substantially or above the height of the superior surface of the outer rope heel sheaves relative to the heel plantar surface. The first universal rope hole sheave may be a first void within the proximal midfoot attachment portion of the forefoot. The void may have two openings on the outer sides of the midfoot attachment portion of the forefoot. The superior surface of the first forefoot universal rope hole sheave may be shaped like a second curved surface portion of a second cylinder. The second cylinder may have a second axis configured substantially parallel to the plantar surface of the forefoot and substantially parallel to the long axis of the forefoot. The distal and proximal walls of the first void function as first universal rope hole sheave rope guides. The midfoot joint may include a first fillet and a second fillet, each between the proximal and distal sides of the first void and the second curved surface of the first forefoot universal rope hole sheave. The width of the first forefoot universal rope hole sheave may be substantially or greater than the cross-sectional diameter of the midfoot universal rope.

The second forefoot universal rope hole sheaves (e.g., midfoot attachment hole upper 187, midfoot attachment hole lower 188, limited twist midfoot twist rope forefoot top sheave 1044, and/or limited twist midfoot twist rope forefoot bottom sheave 1045) may be at substantially or below the height of the inferior surface of the outer rope heel sheaves relative to the heel plantar surface. The second midfoot attachment forefoot sheave may be a second void within the midfoot attachment portion of the forefoot. The second void may have two openings in the outer sides of the midfoot attachment portion of the forefoot. The inferior surface of the second forefoot universal rope sheave may be shaped like a third curved surface portion of a third cylinder. The third cylinder may have a third axis configured substantially parallel to the plantar surface of the forefoot and substantially parallel to the long axis of the forefoot. The proximal and distal walls of the void function as second forefoot universal rope sheave rope guides. And the midfoot joint may include a third fillet and a fourth fillet, each between the proximal and distal sides of the second void and the third curved surface of the second forefoot universal rope hole sheave. The width of the second forefoot universal rope hole sheave may be substantially or greater than the cross-sectional diameter of the midfoot universal rope.

Each outer rope heel sheaves (e.g., limited twist midfoot twist rope heel sheave 2012), midfoot universal rope (e.g., midfoot joint rope 22 and/or limited midfoot twist rope 5008), and first and second forefoot universal rope hole sheaves (e.g., midfoot attachment hole upper 187, midfoot attachment hole lower 188, limited twist midfoot twist rope forefoot top sheave 1044, and/or limited twist midfoot twist rope forefoot bottom sheave 1045) may be configured to allow clearance for the forefoot (e.g., forefoot 12 and/or forefoot 1000) and the heel (e.g., heel 10 and/or heel 2000) to not interfere with each other when the artificial tensegrity midfoot joint (e.g., artificial midfoot joint 7004 and/or artificial midfoot joint 8004) may pass through a selected artificial tensegrity midfoot joint range of motion.

The midfoot joint may include two outer rope heel sheave rope guides (e.g., limited twist midfoot twist rope heel superior guide sheave 2013, limited twist midfoot twist rope inferior guide sheave 2014, midfoot joint outer sheaves 202, and/or limited twist midfoot twist rope heel inferior sheave guide 2016). Each outer rope heel sheave rope guide may protrude radially outward, relative to the fourth axis, from the fourth curved conical surface of each outer rope heel sheave; outer rope heel sheave rope guides may be located distal to the midfoot rope (e.g., midfoot outer sheave distal rope guide 203); each outer rope heel sheave rope guide may be proximal to the midfoot rope (e.g., midfoot outer sheave proximal rope guide 205). The midfoot joint may include fifth and six fillets between the fourth curved conical surface of each outer rope heel sheaves distal to the outer rope heel sheave rope guides and the distal surface of the outer rope heel sheave rope guides.

The midfoot universal rope (e.g., midfoot joint rope 22 and/or limited midfoot twist rope 5008) may be wrapped around the outer rope heel sheaves (e.g., limited twist midfoot twist rope heel sheave 2012) and through the first and the second forefoot universal rope hole sheaves (e.g., midfoot attachment hole upper 187, midfoot attachment hole lower 188, limited twist midfoot twist rope forefoot top sheave 1044, and/or limited twist midfoot twist rope forefoot bottom sheave 1045) at least twice. The outer rope hole sheaves and the first foot universal rope hole sheave and the second forefoot universal rope hole sheave may be at least as wide from proximal to distal as the at least two diameters of the wrapped midfoot universal rope portions.

The joint may be an artificial tensegrity ankle joint (e.g., artificial ankle 7001 and/or artificial ankle 8001). The universal rope may be an ankle universal rope (e.g., ankle universal rope 400). The first compression member may be a heel (e.g., heel 10 and/or heel 2000). The second compression member may be a lower leg (e.g., lower leg 450). The tightener rope may be an ankle tightener rope (e.g., ankle limit rope 414). The ankle universal rope and the ankle tightener rope connect to the heel and to the lower leg in an artificial tensegrity ankle joint. The portion of the artificial tensegrity ankle joint may be a whole tensegrity ankle joint.

The invention may include an ankle tightener rope hole (e.g., ankle limit rope holes 220) of the heel and an ankle tightener rope hole (e.g., lower leg limit rope holes 426) of the lower leg. The ankle tightener rope may be disposed through the ankle tightener rope hole of the heel and the ankle tightener rope of the lower leg, blocking the motion of the heel and the lower leg away from each other. The invention may include a tightener rope adjustment mechanism. The adjustment mechanism may be configured to adjust the length of the tightener rope. Adjusting the length of the tightener rope may alter the tightener rope tension thereby altering the universal rope tension.

The heel (e.g., heel 10 and/or heel 2000) may include first and second proximal heel protrusions. The lower leg (e.g., lower leg 450) may include third and fourth distal lower leg protrusions. The tension member attachment mechanisms may include first and second ankle universal rope holes (e.g., heel universal rope holes 406) in the first and second proximal heel protrusions respectively and third and fourth ankle universal rope holes (e.g., upper joint member universal rope holes 520 and/or lower joint member universal rope holes 522) in the third and fourth distal lower leg protrusions respectively. The first, second, third, and fourth holes may connect to the ankle universal rope. The ankle universal rope may be disposed through the first, second, third, and fourth ankle universal rope holes. The ankle universal rope (e.g., ankle universal rope 400) may be configured to substantially block the motion of the heel and the lower leg toward each other. The first and second proximal heel protrusions and the third and fourth distal lower leg protrusions provide substantial clearance between the heel and lower leg. The heel and lower leg may be able to pivot on the ankle universal rope. The first and second protrusions may not contact the lower leg and the third and fourth protrusions may not contact the heel, thereby providing smooth joint motion.

The invention may include at least one limit rope (e.g., ankle limit rope 414). The limit rope may connect to the lower leg and to the heel. The limit rope may be configured to limit ranges of the multi axial rotation when the limit rope is in a taut configuration. The at least one of the limit ropes may be prestretched. Substantially no further joint motion may occur in the direction opposed by the limit rope when the limit rope is in a taut configuration.

The ankle limit rope (e.g., ankle limit rope 414) may be connected to the heel (e.g., heel 10 and/or heel 2000) and to the lower leg (e.g., lower leg 450). The tension member attachment mechanisms may include a first ankle limit rope hole (e.g., ankle limit rope hole 220) in the heel and a second ankle limit rope hole (e.g., lower leg limit rope holes 426) in the lower leg for may connect to the ankle limit rope. The ankle limit rope may be disposed through the first and second ankle limit rope holes. The ankle limit rope may be configured to substantially block the motion of the artificial ankle joint in the plantarflexion direction or dorsiflexion direction when the ankle limit rope is in a taut configuration.

The at least one limit rope may be elastic. The joint may stop compliantly as a result of engagement of the elastic limit rope. After engagement of the elastic limit rope, the elastic limit rope may allow for the return of energy to one of the compression members as the joint returns to substantially the same position it was in prior to the engagement of the elastic limit rope. The joint may absorb an impact shock upon rapid motion of the compression members. The limit ropes may be configured to be adjustable, and may thereby alter the range of motion of the joint.

At least one stabilizer rope (e.g., ankle stabilizer rope 402) may connect to the lower leg (e.g., lower leg 450) and to the heel (e.g., heel 10 and/or heel 2000) and may be configured to substantially block directions of the multi axial rotation. The at least one stabilizer rope may be elastic and may allow the heel and lower leg to pivot on the ankle universal rope (e.g., ankle universal rope 400). The lower leg may include a lower leg stabilization rope hole (e.g., lower leg stabilization rope hole 412). The heel may include a heel stabilization rope hole (e.g., heel stabilization rope hole 408).

The ankle stabilization rope (e.g., ankle stabilizer rope 402) may be connected to the heel and to the lower leg. The ankle stabilization rope may be disposed through the lower leg stabilization rope hole and through the heel stabilization rope hole. The ankle stabilization rope may be configured to substantially block the range of motion of the artificial ankle joints (e.g., artificial ankle 7001 and/or artificial ankle 8001). The range of motion may be substantially blocked in a direction of at least one of eversion and inversion directions.

The artificial tensegrity ankle joint may have substantially the same or better structural strength compared to the corresponding natural joint as measured in terms of the torque/response curve or ultimate compressive strength.

The invention may include an adjustment mechanism may be configured to limit the range of motion of the lower leg substantially the heel by ceasing motion of the lower leg in either one of the flexion and extension directions. The adjustment mechanism may be configured to adjust the lengths of at least one of the dorsiflexion limit rope (e.g., extension limit rope 38, dorsiflexion limit rope 62, and/or MTP dorsiflexion limit rope 5014) and the plantarflexion limit rope (e.g., flexion limit rope 40, plantarflexion limit rope 54, and/or MTP plantarflexion limit rope 5012).

The invention may include an anti-twist mechanism configured to substantially block the twisting motion of the lower leg portion relative to the heel. The invention may include an attachment mechanism may connect the heel to an artificial knee joint or a natural or artificial leg portion. An attachment mechanism may connect the heel and at least one component. The component may include at least one of an midfoot joint, a foot, an MTP joint, and toes. An attachment mechanism may connect the leg to a natural or artificial proximal anatomic structure.

The attachment of the ankle universal rope (e.g., ankle universal rope 400), the lower leg (e.g., lower leg 450), and the heel (e.g., heel 10 and/or heel 2000) may create two virtual axes of motion. The first ankle axis may be perpendicular to the face of the ankle attachment stubs of the heel that contains the ankle universal rope hole (e.g., heel universal rope hole 406) of the heel, slightly below the level of the ankle universal rope holes of the heel, and substantially between the two ankle attachment stubs. A second ankle axis may be perpendicular to face of the lower leg that contains the ankle universal rope hole of the lower leg, slightly higher than the level of the ankle universal rope holes of the lower leg, and substantially between the two ankle universal rope holes of the lower leg.

A range of motion of the artificial ankle joint may move, from substantially maximal excursion in the plantarflexion direction to substantially maximal excursion in the dorsiflexion direction, may be between about 0.1 degrees and about 120 degrees, about 0.1 degrees and about 90 degrees, about 0.5 degrees and about 60 degrees, about 1 degree and about 30 degrees, and about 1 degree and about 10 degrees around the first ankle axis.

A range of motion of the artificial ankle joint may move, from substantially maximal excursion in the eversion direction to substantially maximal inversion in the extension direction, may be between about 0.1 degrees and about 35 degrees, about 0.1 degrees and about 15 degrees, and about 0.5 degrees and about 5 degrees around the second ankle axis.

The invention may include at least one of heel universal rope holes (e.g., heel universal rope holes 406), at least one heel stabilizer rope holes (e.g., heel stabilization rope hole 408), at least one lower leg universal rope hole (e.g., lower leg universal rope hole 410), at least one lower leg stabilization rope hole (e.g., lower leg stabilization rope hole 412), at least one lower leg limit rope hole (e.g., lower leg limit rope hole 426), and sheaves (e.g., ankle sheaves 502) with an attachment mechanism that may connect sheaves to the heel or the lower leg portion.

The invention may include at least one ankle actuator. The ankle actuator may connect to at least of the lower leg (e.g., lower leg 450), the heel (e.g., heel 10 and/or heel 2000), the lower leg portion. The at least one of the actuators may be powered. The ankle actuator may be an actuator that spans from the first ankle joint compression member to the second ankle joint compression member, for example, from the heel (e.g., heel 10 and/or heel 2000) to the lower leg (e.g., lower leg 450). The ankle actuator may be an actuator that is directly involved in creating and/or altering ankle motion.

The artificial joint may be at least a portion of an artificial tensegrity kn knee joint (e.g., artificial knee 7002). The artificial tensegrity knee joint may include a third compression member (e.g., x-brace 500, upper joint member 504 and/or lower joint member 506) and a second universal rope (e.g., upper knee universal rope 508 and/or lower knee universal rope 510). A second set of at least one rope attachment mechanisms may connect the second universal rope to the third compression member. The third compression member may be a distal leg portion (e.g., upper joint member 504 and/or lower joint member 506). The second universal rope may be a distal knee universal rope (e.g., upper knee universal rope 508 and/or lower knee universal rope 510). The first universal rope may be a proximal knee universal rope (e.g., upper knee universal rope 508 and/or lower knee universal rope 510). The first compression member may be a proximal leg portion (e.g., upper joint member 504 and/or lower joint member 506). The second compression member may be an x-brace (e.g., x-brace 500). The tightener rope may be a knee tightener rope (e.g., knee tightener rope 528). The second set of rope attachment mechanisms may connect the second knee universal rope to the x-brace and to the distal leg portion. The artificial tensegrity knee joint may be a whole tensegrity knee joint (e.g., artificial knee 7002).

The invention may include at least one of a knee tightener rope hole (e.g., x-brace tightener rope hole 530) of the x-brace, a knee tightener rope hole (e.g., upper joint member tightener rope hole 532 and/or lower joint member tightener rope hole 534) of the proximal leg, and a knee tightener rope hole (e.g., upper joint member tightener rope hole 532 and/or lower joint member tightener rope hole 534) of the distal leg. The knee tightener rope may be disposed to pass through the knee tightener rope hole of the x-brace, through knee tightener rope hole of the proximal leg,and through the knee tightener rope hole of the distal leg, blocking the motion of the x-brace, the distal leg, and the proximal leg away from each other.

The invention may include a tightener rope adjustment mechanism. The adjustment mechanism may be configured to adjust the length of the knee tightener rope. Adjusting the length of the tightener rope may alter the knee tightener rope tension, thereby adjusting the tension in the first and second knee universal ropes.

The proximal knee universal rope may be configured to create two virtual axes of motion. A first knee axis may lie in substantially the same plane as the x-brace, parallel to and slightly proximal to a line that may connect two x-brace first universal rope holes. A second knee axis may be substantially perpendicular to the first knee axis, slightly proximal to a line that may connect two proximal leg first universal rope holes. A range of motion of the artificial upper knee joint, from substantially maximal excursion in the flexion direction to substantially maximal excursion in the extension direction, may be between at least one of about 0.1 degrees and about 185 degrees, about 0.5 degrees and about 120 degrees, about 1 degree and about 90 degrees, and about 1 degree and about 60 degrees around the first knee axis.

At least one adjustment mechanism may be configured to limit the ranges of motion of the distal leg and the proximal leg or leg portion substantially the x-brace by ceasing motion of the distal leg and proximal leg in the flexion and extension directions. The adjustment mechanism may be configured to adjust the lengths of at least one of the extension limit rope and the flexion limit rope. An attachment mechanism may connect the lower leg to an artificial ankle joint or a natural or artificial leg portion.

At least one stabilizer rope (e.g., upper knee stabilization rope 512, and/or lower knee stabilization rope 514) may connect to at least two of distal I distal leg portion (e.g., upper joint member 504 and/or lower joint member 506), proximal distal leg portion (e.g., upper joint member 504 and/or lower joint member 506), and x-brace (e.g., x-brace 500). The stabilizer ropes may be configured to substantially block directions of the multi axial rotation of the tensegrity knee joint. The at least one stabilizer rope may be elastic and may allow the compression members to pivot on the proximal or distal universal rope (e.g., upper knee universal rope 508 and/or lower knee universal rope 510).

A distal leg portion may include a distal leg portion stabilization rope hole (e.g., upper joint member stabilization rope hole 532 and/or lower joint member stabilization rope hole 534). The x-brace may include an x-brace stabilization rope hole (e.g., x-brace stabilization rope hole 518). The proximal leg portion may include a proximal leg portion stabilization rope hole (e.g., upper joint member stabilization rope hole 532 and/or lower joint member stabilization rope hole 534). The knee stabilizer rope (e.g., upper knee stabilization rope 512, and/or lower knee stabilization rope 514) may be connected to at least at least of the distal leg portion, and the proximal leg portion. The knee stabilization rope may be disposed through the the x-brace stabilization rope hole and a second stabilization rope hole may be at least one of the distal leg portion stabilization rope hole and the proximal leg portion stabilization rope hole. The knee stabilization rope may be configured to substantially block the range of motion of the knee.

The knee limit rope (e.g., knee tightener rope 528) may be connected to at least two of the x-brace (e.g., x-brace 500), the distal leg portion (e.g., upper joint member 504 and/or lower joint member 506), and the proximal leg portion (e.g., upper joint member 504 and/or lower joint member 506). The tension member attachment mechanisms may include a first knee limit rope hole (e.g., upper joint member tightener rope hole 532 and/or lower joint member tightener rope hole 534) in the first compression member and a second knee limit rope hole (e.g., upper joint member tightener rope hole 532 and/or lower joint member tightener rope hole 534) in the second compression member. The tension member attachment mechanisms may connect to the knee limit rope. The knee limit rope may be disposed through the first and second knee limit rope holes. The knee limit rope may be configured to substantially block the motion of the artificial knee joint in the flexion direction or extension direction when the knee limit rope is in a taut configuration.

The invention may include least one limit rope (e.g., knee tightener rope 528). The limit rope may be connected to at least two of the lower leg, the upper leg, and the x-brace. The limit ropes may be configured to limit ranges of the multi axial rotation when pulled taut. The at least one limit rope may be elastic. The joint may stop compliantly as a result of engagement of the elastic limit rope. After engagement of the elastic limit rope, the elastic limit rope may allow for the return of energy to one of the compression members as the joint returns to substantially the same position it was in prior to the engagement of the elastic limit rope. The joint may absorb an impact shock upon rapid motion of the compression members. The at least one of the limit ropes may be prestretched. Substantially no further joint motion may occur in the direction opposed by the limit rope when the limit rope is in a taut configuration. The length of the limit ropes may be configured to be adjustable, and may thereby alter the range of motion of the joint.

The distal knee universal rope (e.g., upper knee universal rope 508 and/or lower knee universal rope 510) may be configured to create two virtual axes of motion. A third knee axis may lie in substantially the same plane as the x-brace, parallel to and slightly proximal a line that may connect two x-brace second universal rope holes. A fourth knee axis may be substantially perpendicular to the third knee axis, slightly proximal to a line that may connect two distal leg second universal rope holes. The range of motion of the artificial lower knee joint, from substantially maximal excursion in the flexion direction to substantially maximal excursion in the extension direction, may be between at least one of about 0.1 degrees and about 185 degrees, about 0.5 degrees and about 120 degrees, about 1 degree and about 90 degrees, and about 1 degree and about 60 degrees around the third knee axis.

The x-brace (e.g., x-brace 500) may include first and second proximal knee protrusions. The proximal leg portion (e.g., upper joint member 504 and/or lower joint member 506) may include third and fourth proximal knee protrusions. The tension member attachment mechanisms may include first and second knee universal rope holes (e.g., x-brace universal rope holes 516) in the first and second proximal knee protrusions respectively and third and fourth knee universal rope holes (e.g., upper knee universal rope hole 520 and/or lower knee universal rope hole 522) in the third and fourth knee protrusions respectively. The first, second, third, and fourth holes may connect to the first knee universal rope (e.g., upper knee universal rope 508 and/or lower knee universal rope 510). The first knee universal rope may be disposed through the first, second, third, and fourth knee universal rope holes. The first knee universal rope may be configured to substantially block the motion of the x-brace and the proximal leg portion toward each other. The first and second knee protrusions and the third and fourth knee protrusions may be configured to provide substantial clearance between the x-brace and the proximal leg portion. The x-brace and proximal leg portion may be able to pivot on the first knee universal rope. The first and second knee protrusions may not contact the proximal leg portion and the third and fourth protrusions may not contact the x-brace, thereby providing smooth joint motion within the specified range of motion.

The x-brace (e.g., x-brace 500) may include fifth and sixth distal knee protrusions. The distal leg (e.g., upper joint member 504 and/or lower joint member 506) may include seventh and eighth distal knee protrusions. The tension member attachment mechanisms may include fifth and sixth knee universal rope holes (e.g., x-brace universal rope holes 516) in the fifth and sixth distal knee protrusions respectively and seventh and eighth knee universal rope holes (e.g., upper knee universal rope hole 520 and/or lower knee universal rope hole 522) in the seventh and eighth knee protrusions respectively. The fifth, sixth, seventh and eighth holes may connect to the second knee universal rope (e.g., upper knee universal rope 508 and/or lower knee universal rope 510). The second knee universal rope may be disposed through the fifth, sixth, seventh, and eighth knee universal rope holes. The second knee universal rope may be configured to substantially block the motion of the x-brace and the distal leg toward each other. The fifth and sixth knee distal protrusions and the seventh and eighth distal knee protrusions may be configured to provide substantial clearance between the x-brace and the distal leg. The x-brace and distal leg may be able to pivot on the second knee universal rope, and the fifth and sixth knee protrusions may not contact the distal leg and the seventh and eighth protrusions may not contact the x-brace, thereby providing smooth joint motion within the specified range of motion. The artificial tensegrity knee joint may have similar or better structural strength compared to the corresponding natural joint as measured in terms of the torque/response curve or ultimate compressive.

The invention may include an anti-twist mechanism configured to substantially block the twisting motion of the at least one of the first, second, and third compression members relative to the remaining knee joint compression members.

The invention may include at least two of the artificial tensegrity universal joints stacked on top of each other, for example, as shown in FIGS. 20 and 21A-21B. The invention may include an attachment mechanism that may connect the upper leg to a natural or artificial proximal anatomic structure.

The invention may include at least one knee actuator that may connect to at least one of the distal leg, the proximal leg, the natural or artificial proximal leg portion (e.g., upper joint member 504 and/or lower joint member 506), and/or the x-brace (e.g., x-brace 500). The at least one knee actuator may be powered. For example, the knee actuator may span from the lower leg (e.g., lower joint member 506) to the x-brace (e.g., x-brace 500), from the upper leg (e.g., upper joint member 504) to the x-brace (e.g., x-brace 500), or from the lower leg (e.g., lower joint member 506) to the upper leg (e.g., upper joint member 504). In another example, the knee actuator may be directly involved in creating or altering knee motion.

The invention may include at least one of an x-brace (e.g., x-brace 500), universal rope holes (e.g., x-brace universal rope holes 516), x-brace stabilization rope hole (e.g., x-brace stabilization rope hole 518), proximal joint member universal rope holes (e.g., upper knee universal rope hole 520 and/or lower knee universal rope hole 522), distal joint member universal rope holes (e.g., upper knee universal rope hole 520 and/or lower knee universal rope hole 522), proximal joint member stabilization rope holes (e.g., upper joint member stabilization rope hole 532 and/or lower joint member stabilization rope hole 534), distal joint member stabilization rope holes (e.g., upper joint member stabilization rope hole 532 and/or lower joint member stabilization rope hole 534), knee tightener rope knee tightener rope (e.g., knee tightener rope 528), x-brace tightener rope hole (e.g., x-brace tightener rope hole 530), proximal joint member tightener rope hole (e.g., upper joint member tightener rope hole 532 and/or lower joint member tightener rope hole 534), distal joint member tightener rope hole (e.g., upper joint member tightener rope hole 532 and/or lower joint member tightener rope hole 534), and sheaves (e.g., knee sheaves 502) with an attachment mechanism that may connect sheaves to the x-brace, distal leg or proximal leg portion.

A further embodiment of the invention may include at least a portion of a prosthetic or orthotic foot device for a human, or a robotic foot. The device may include at least one artificial toe (e.g., big toe 14, middle toe 16, little toe 18, and/or toe (wide) 3000), an artificial forefoot (e.g., forefoot 12 and/or forefoot 1000), an artificial heel (e.g., heel 10 and/or heel 2000), at least one artificial metatarsophalangeal (MTP) joint (e.g, artificial MTP joint 7003 and/or artificial MTP joint 8003), an artificial midfoot joint (e.g., artificial midfoot joint 7004 and/or artificial midfoot joint 8004), and a coordination mechanism (e.g., cross-joint coordination rope 5018) configured to coordinate the motion of at least one of the MTP joints and the subtalar joint. The coordinated motion may correspond to the motion of the natural foot during ambulation. The MTP joint may connect the at least one artificial toe and the forefoot. The midfoot joint may connect the forefoot and the heel. In various embodiments, the invention may include one or more of any of the aspects set forth herein.

The device may fit in a standard shoe. The MTP and the midfoot motion may be coordinated by mechanical coupling. The invention may include at least one actuator (e.g., toe actuator 36). Each of the at least one actuators may connect to at least one of the toes and to the heel. The at least one actuator may be powered.

The forefoot may include a mechanism to adjust the length of the forefoot, as measured along the long axis of the foot. The mechanism may include at least one of rack and pinion mechanisms, ratchet mechanisms, hole and pin mechanisms, ball and detente adjustment mechanisms, and other length adjustment mechanisms. The forefoot length adjustment mechanism may include at least one of the adjustment mechanisms. The adjustment mechanisms may include at least one of rack and pinion mechanisms, ratchet mechanisms, hole and pin mechanisms, and ball and detente adjustment mechanisms. The attachment mechanism may attach the foot to an artificial ankle joint. The attachment mechanism may attach the foot to a leg portion of the human or robot.

The forefoot may have at least one angled walking surface (e.g., medial angled walking surface 160, lateral angled walking surface 162, angled walking surface 304, and/or walking surface 1006) and may be configured to roll on the angled walking surface. One of the toes may be a lateral toe. A range of motion of the lateral toe may be configured to substantially block the rolling motion and redirect the rolling motion toward the medial side of the foot.

The mechanism may be configured to coordinate the motion of at least one of the MTP joints and the subtalar joint, such that when weight is backed off of the toes by a contralateral leg heel strike, the mechanism may respond with a spring-like action, pulling the midfoot/subtalar joint into a substantially neutral configuration, and may release energy that propels the foot forward and into swing phase.

The invention may include at least one actuator. Each of the at least one actuators may connect to at least one of the toes 3000 and to the forefoot 12, 1000. The at least one of the actuator may be powered.

The invention may include at least one of screw swages 42, stop swages 44, stop swage keeper 46, bottom rope keeper 48, rope guide keepers 50, twist rope 52, plantarflexion limit rope 54, plantarflexion limit rope sheave, plantarflexion limit rope guide, round midfoot outer sheaves (e.g., midfoot joint outer sheaves 202), tapered midfoot joint outer sheaves 212, cushion cavity 214, coordination rope attachment 216, angled plate mount for sheaves 218, a means for attaching the components to the toes and/or the forefoot or means may attach each of the components to the heel and/or forefoot, toe supports 102, front sheaves 104, an axial rope hole 106, a tall axial sheave 108, a short axial sheave 110, an axial sheave rope keeper flange 111, a spacer for plantarflexion ropes 112, short spacer 114, dorsiflexion limit rope holes 116, open dorsiflexion limit rope holes 118, plantarflexion limit rope holes 120, open plantarflexion limit rope holes 122, dorsiflexion limit rope guides 124, limit rope guides 126, a plantarflexion limit rope guide trim 128, a plantarflexion limit rope spreader 130, rope guide fillets 132, a dorsiflexion limit rope spreader 136, stiffener bars 152, a medial angled walking surface 160, a lateral angled walking surface 162, a screw attachment bar 164, a midfoot joint attachment 168, a clearance for midfoot joint 172, a first midfoot rope hole 176, a second midfoot rope hole 178, a forefoot actuator stays 180, a tightener rope hole 182, a rear weight platform 184, an axial rope screw hole attachment 185, an axial rope swage loop attachment hole 186, a first midfoot attachment hole 187, a second midfoot attachment hole 188, a plantarflexion limit rope sheave 189, a sheave structural support 190, a plantarflexion limit rope guide 191, a plantarflexion sheave surface 192, a cushioned heel 200, midfoot joint outer sheaves 202, a Movement Clearance 204, a pylon 206, a tightener rope hole 210, a heel actuator stay 211, tapered midfoot joint outer sheaves 212, a heel cushion cavity 214, a coordination rope 215, a heel coordination rope attachment 216, an angled plate 218, ankle limit rope holes 220, a front side sheave 300, a front top sheave 302, an angled walking surface 304, a main column 306, an axial rope hole 312, an actuator hook 316, a toe sheave 330, a Bearing Clearance 334, a thick web 338, a thin web 340, a top beam 342, a toe race 380, an inner curve 382, and bearings 383. At least one actuator may connect to the heel and to the forefoot. The at least one actuator may be powered. The heel may be cushioned.

The invention may include toes (e.g., big toe 14, middle toe 16, little toe 18, and/or toe (wide) 3000) of different sizes. One of the toes may be a big toe (e.g., big toe 14) on the medial side of the foot. One of the toes may be a little toe (e.g., little toe 18) on the lateral side of the foot. The one big toe, at least one medium toe (e.g., middle toe 16), and/or one small toe may be so arranged medially to laterally, respectively. An attachment mechanism may connect the heel to an ankle joint or to a natural or artificial leg portion.

The artificial MTP joint (e.g, artificial MTP joint 7003 and/or artificial MTP joint 8003) may include a tensegrity joint. The artificial midfoot joint (e.g., artificial midfoot joint 7004 and/or artificial midfoot joint 8004) may include a tensegrity joint. The coordination mechanism may be a tension or a compression member connected to one or more of the artificial toes (e.g., big toe 14, middle toe 16, little toe 18, and/or toe (wide) 3000) and to the artificial heel (e.g., heel 10 and/or heel 2000). The compression member may be configured to cause the toes and the heel to move substantially synchronously. The tension member may be configured to cause the toes and the heel to move substantially synchronously when the tension member is in a taught configuration. The portion of the prosthetic, orthotic, or a robotic foot may be a whole prosthetic, orthotic, or a robotic foot.

Yet another embodiment of the invention may include at least a portion of an artificial tensegrity joint. The artificial tensegrity joint may include a first compression member, a second compression member, at least one tension member including, and at least one twist tension member (e.g., twist rope 52 and/or limited midfoot twist rope 5008). The at least one twist tension member may connect the first compression member and the second compression member to each other.

The invention may include a second tension member. The second tension member may include at least one limiting tension member (e.g., axial rope 28, extension limit rope 38, flexion limit rope 40, plantarflexion limit rope 54, dorsiflexion limit rope 62, ankle limit rope 414, Achilles limit rope 5010, MTP plantarflexion limit rope 5012, MTP dorsiflexion limit rope 5014, and/or MTP axial rope 5016). The at least one limiting tension member may connect the first compression member and the second compression member to each other. At least one attachment mechanism may connect a tension member to at least one of the first compression member, the second compression member, and itself.

A yet further embodiment of the invention may include an artificial tensegrity limited twist midfoot joint (e.g., artificial midfoot twist joint 7006 and/or artificial midfoot twist joint 8006). The artificial tensegrity limited twist midfoot joint may include the first compression member which may include a forefoot (e.g., forefoot 12 and/or forefoot 1000). The forefoot may include a midfoot attachment portion (e.g., midfoot joint attachment 168 and/or midfoot attachment portion 1004). The forefoot may include a plantar surface (e.g., plantarflexion sheave surface 192). The second compression member may include a heel (e.g., heel 10 and/or heel 2000). The twist tension member may include a twist rope (e.g., twist rope 52 and/or limited midfoot twist rope 5008). The limiting tension member may include at least one plantar rope 54, 5006 and at least one Achilles rope 5010. The at least one attachment mechanism may connect the twist rope the plantarflexion limit rope (e.g., flexion limit rope 40, plantarflexion limit rope 54, and/or MTP plantarflexion limit rope 5012) and the Achilles limit rope (e.g., ankle limit rope 414 and/or Achilles limit rope 5010) to the heel and to the forefoot.

The ropes may be configured to pull taut at the same time when the joint may be at the maximum range of motion. The joint may be bilaterally symmetric about the midsaggital plane of the foot.

The artificial tensegrity limited twist midfoot joint may include at least one sheave. The at least one sheave may include two limited midfoot twist rope heel sheaves. The twist rope may include a midfoot twist rope. The midfoot twist rope may be disposed around each of the two limited midfoot twist rope heel sheaves. The midfoot twist rope may be configured to substantially block the rotation of the artificial tensegrity limited twist midfoot joint in the dorsiflexion direction when the midfoot twist rope is in a taut configuration about the two limited midfoot twist rope heel sheaves.

The invention may include a limited twist rope complex. The limited twist rope complex two limited midfoot twist rope heel sheaves (e.g., limited midfoot twist rope heel sheave 2012), two limited twist rope forefoot sheaves (e.g., limited twist rope forefoot sheaves 1040), one limited midfoot twist rope forefoot top sheave (e.g., limited twist rope forefoot top sheave 1044), and one limited midfoot twist rope forefoot bottom sheave (e.g., limited twist rope forefoot bottom sheaves 1045). The limited twist rope complex (e.g., limited twist rope complex 5002) may be configured to allow the limited twist midfoot rope (e.g., limited midfoot twist rope 5008) to connect the forefoot and the heel and to provide an axis for the forefoot and the heel to rotate around. Each of the two limited midfoot twist rope heel sheaves may be substantially C shaped. The limited midfoot twist rope heel sheaves may extend from the front most face of the heel. The two limited midfoot twist rope heel sheaves may be substantially mirror images of each other. Each limited midfoot twist rope heel sheave C-shape opening may substantially face toward the mid-sagital plane of the heel.

Each limited midfoot twist heel sheave may include a superior sheave (e.g., limited twist midfoot twist rope heel superior guide sheave 2013) and an inferior sheave (e.g., limited twist midfoot twist rope heel inferior guide sheave 2014). Each superior sheave may include a first curved surface shaped substantially like a first longitudinal half cylinder curved surface. Each inferior sheave may include a second curved surface shaped substantially like a second longitudinal half cylinder curved surface. Each superior sheave first curved surface may face in the superior direction. Each inferior sheave second curved surface may face in the inferior direction. Each first and second longitudinal half cylinder curved surface may have a first axis substantially parallel to the long axis of the forefoot. A line tangent to the outermost edges of the first and second curved surface of the superior and inferior sheaves, respectively, may be substantially perpendicular to the plantar surface of the forefoot. Third and fourth inferior surface portions of each superior sheave may substantially contact a first portion of each of the two limited twist midfoot rope forefoot sheaves for transferring weight from the heel to the forefoot to a surface below. Each superior sheave medial edge may be sufficiently medial to the most lateral end of each limited twist midfoot rope forefoot sheave to prevent the twist rope from slipping off of the lateral edge of the limited twist midfoot rope forefoot sheave. Sufficiently medial may be defined as close enough to the center of the foot such that when the rope wraps over the top of the superior sheave and then travels down and around the twist rope forefoot sheave, it may be directed onto the twist rope forefoot sheave, not off the lateral end of the twist rope forefoot sheave. The uncontacted second portion of the limited twist midfoot rope forefoot sheaves between the midfoot attachment portion of the forefoot and the medial surface of each limited midfoot twist rope heel sheave may be long enough for the selected number of may wrap of the twist midfoot rope.

The third and fourth inferior surfaces may be substantially flat. The third and fourth inferior surface may angle upwards towards the mid plane of the forefoot 12, 1000.

The invention may include a fillet. Each limited midfoot twist rope heel sheaves midfoot twist rope heel sheaves (e.g., limited twist midfoot twist rope heel sheave 2012, limited twist midfoot twist rope heel superior guide sheave 2013, limited twist midfoot twist rope inferior guide sheave 2014, and/or limited twist midfoot twist rope superior sheave 2017) may be filleted where the inferior surface of the superior sheave is adjacent to the more inferior medial surface of the limited midfoot twist rope heel sheave. Each limited midfoot twist rope heel sheave may include a superior guide sheave (e.g., limited twist midfoot twist rope heel superior guide sheave 2013) and an inferior guide sheave (e.g., limited twist midfoot twist rope heel inferior guide sheave 2014). The superior and inferior guide sheaves may be configured to limit front and back movement of the twist rope (e.g., twist rope 52 and/or limited midfoot twist rope 5008). The superior guide sheave and inferior guide sheave may be located on the lateral side of the limited midfoot twist rope heel sheave. The superior and inferior sheave guide sheaves may extend laterally from the limited midfoot twist rope heel sheave Each limited midfoot twist rope heel guide sheave may include a front portion and a back portion. The rearmost edge of the superior guide sheave (e.g., rear edge of the superior guide sheave 2019) may be shaped like the graph of the mathematical tangent function. The front-most edge of the inferior guide sheave (e.g., front-most edge of the inferior guide sheave 2015) may be shaped like the graph of the mathematical tangent function. The space between the front-most edge of the inferior guide sheave and the rearmost edge of the superior guide sheave may be substantially equal to or more than the width of the midfoot twist rope.

The invention may include at least two inferior sheave guides (e.g., limited twist midfoot twist rope heel inferior sheave guide 2016). Each inferior sheave guide may extend radially in the plantar and medial and lateral directions from the 2nd cylinder axis at the front most edge of the inferior sheave. The heel may include two superior faces adjacent to the extension of the limited midfoot twist rope heel sheaves which function as superior sheave guides on the back side of the superior sheave guide.

The invention may include a limited midfoot twist rope forefoot bottom sheave (e.g., limited twist midfoot twist rope forefoot bottom sheave 1045). The limited midfoot twist rope forefoot bottom sheave may protrude rearward from the rearmost portion of the midfoot attachment portion (e.g., midfoot joint attachment 168 and/or midfoot attachment portion 1004) of the forefoot. The limited midfoot twist rope forefoot bottom sheave may be centered substantially on the midline of the forefoot. The limited midfoot twist rope forefoot bottom sheave may be at least long enough to accommodate the specified number of wrapsof the midfoot Achilles rope (e.g., ankle limit rope 414 and/or Achilles limit rope 5010). The inferior face of the limited midfoot twist rope forefoot bottom sheave may be shaped like substantially a second half longitudinal cylindrical surface. The second cylindrical surface may have a second axis that may be parallel to the long axis of the forefoot.

The invention may include a limited midfoot twist rope forefoot top sheave guide (e.g., limited twist midfoot twist rope forefoot top sheave 1044). The limited midfoot twist rope forefoot top sheave may be centered substantially on the midline of the forefoot. The limited midfoot twist rope forefoot top sheave may be at least long enough to accommodate the specified number of wrapsof the midfoot Achilles rope (e.g., ankle limit rope 414 and/or Achilles limit rope 5010). The limited midfoot twist rope forefoot top sheave may be shaped like substantially a second half longitudinal cylindrical surface. The second cylindrical surface may have a second axis that may be in-plane with the midplane of the forefoot and may be perpendicular to a line that may be tangent to the surface of the limited twist midfoot twist rope forefoot sheave. Rope guides may be present on front and rear side of the sheave, and the sheave may be filleted with rope guides.

The two limited midfoot twist rope forefoot sheaves (e.g., limited twist rope forefoot sheaves 1040, limited twist rope forefoot top sheave 1044, and/or limited twist rope forefoot bottom sheaves 1045) may be each shaped like a cylinder, with a first axis perpendicular to the long axis of the forefoot, and parallel to the plantar surface of the forefoot. The two limited midfoot twist rope forefoot sheaves may extend outward from the midfoot attachment portion of the forefoot. Each limited midfoot twist rope forefoot sheave may extend lateral enough to accommodate the selected number of wraps of the midfoot rope (e.g., ankle limit rope 414 and/or Achilles limit rope 5010) and to may extend sufficiently more laterally than the most medial edge of each limited midfoot twist rope heel sheaves to allow the twist rope to transfer between the limited midfoot twist rope heel sheave and the limited midfoot twist rope forefoot sheave far enough from the lateral edge of the limited twist midfoot rope forefoot sheave to prevent the twist rope (e.g., twist rope 52 and/or limited midfoot twist rope 5008) from slipping off. Each limited midfoot twist rope forefoot sheave may be short enough so that the lateral most surface may not contact the surface of the limited midfoot twist rope heel sheaves at the height of the first axis. A portion of the superior surface of the limited midfoot twist rope forefoot sheave may not contact the limited midfoot twist rope heel sheave to transfer weight from the heel to the forefoot to a surface below. There may be compressive loading at the contact point between the limited midfoot twist rope heel sheaves and the limited midfoot twist rope forefoot sheaves. The limited midfoot twist rope forefoot sheaves may be substantially mirror images of each other.

The invention may include a fillet. Each limited midfoot twist rope forefoot sheave may be filleted lateral to the point of contact with the limited midfoot twist rope heel sheave to have a similar shape as the nearest portion of the limited midfoot twist rope heel sheave.

The invention may include a limited twist rope path. The limited twist rope path may dispose the twist rope (e.g., twist rope 52 and/or limited midfoot twist rope 5008) toward the rear of the forefoot (e.g., forefoot 12 and/or forefoot 1000) by guiding the twist rope toward the rearward part of the superior substantially-semi-cylindrical portion of the limited midfoot twist rope heel sheave (e.g., limited twist midfoot twist rope heel sheave 2012, limited twist midfoot twist rope heel superior guide sheave 2013, and/or limited twist midfoot twist rope inferior guide sheave 2014). The limited twist rope path may dispose the twist rope toward the front of the forefoot by guiding the twist rope toward the forward part of the inferior substantially-semi-cylindrical portion limited midfoot twist rope heel sheave. A front and/or back surface of the limited midfoot twist rope heel sheave guides may function as sheaves as they guide the rope toward the superior-rear region and the inferior-front region of the substantially vertical part of the “C” shaped limited midfoot twist rope heel sheave. The twist rope may be disposed over and around the superior sheave and down between the limited midfoot twist rope heel sheave guides to wrap around the limited midfoot twist rope forefoot sheave such that the twist rope may be disposed around and up the inferior substantially-semi-cylindrical face, for example, in order to wrap around the limited midfoot twist rope forefoot sheave.

The midfoot twist rope (e.g., twist rope 52 and/or limited midfoot twist rope 5008) may be disposed from the limited midfoot twist rope forefoot bottom sheave (e.g., or limited twist rope forefoot bottom sheaves 1045) around the superior sheave (e.g., limited twist midfoot twist rope heel superior guide sheave 2013). The midfoot twist rope may be disposed along the back face of the superior guide sheave on the superior lateral portion of the midfoot twist rope heel sheave. The midfoot twist rope may be disposed along the lateral side of the limited midfoot twist rope heel guide sheave between the between the superior and inferior guide sheaves. The midfoot twist rope may be disposed along the front face of the inferior guide sheave (e.g., limited twist midfoot twist rope inferior guide sheave 2014 and/or limited twist midfoot twist rope inferior sheave 2018) on the inferior lateral portion of the limited midfoot twist rope heel sheave. The midfoot twist rope may be disposed around the inferior sheave while guided by the inferior sheave guide.

The invention may include a limited Achilles rope complex. The limited Achilles rope complex may include a limited midfoot Achilles rope heel sheave (e.g., limited twist midfoot Achilles rope heel sheave 2020) and a limited midfoot Achilles rope forefoot sheave (e.g., midfoot Achilles rope forefoot sheave 1036). The limited Achilles rope complex may be configured to allow the Achilles rope (e.g., ankle limit rope 414 and/or Achilles limit rope 5010) to substantially block motion away from each other of the limited midfoot Achilles rope heel sheave and the limited midfoot Achilles rope forefoot sheave. The motion away from each other that is substantially blocked may be along the axis perpendicular to the plantar surface of the forefoot 12, 1000 when pulled taut.

The heel may include a U-shaped main body. The limited midfoot Achilles rope heel sheave may protrude rearward from the rearmost part of the U-shaped heel body. The limited midfoot Achilles rope heel sheave may be centered substantially on the midsagital plane of the heel. The limited midfoot Achilles rope heel sheave may be at least long enough to accommodate the specified number of may wrap of the midfoot Achilles rope. The superior face of the limited midfoot Achilles rope heel sheave may be shaped like substantially a first half longitudinal cylindrical surface. The first cylindrical surface may have an axis that may be parallel to the long axis of the forefoot. The limited midfoot Achilles rope heel sheave may include a hole (e.g., limited twist midfoot Achilles rope heel stop hole 2021 and/or limited twist midfoot Achilles rope heel screw hole 2024). The hole may be in the direction substantially perpendicular to the plantar surface of the forefoot. The hole may extend completely through the limited midfoot Achilles rope heel sheave.

The invention may include a terminal screw end fitting. The terminal screw end fitting may be disposed through the hole. The terminal screw end fitting may be configured to may extend in the direction opposite the heel plantar surface and block the motion of the midfoot Achilles rope in the rearward direction.

The limited midfoot Achilles rope heel sheave (e.g., limited twist midfoot Achilles rope heel sheave 2020) may include a limited midfoot Achilles heel sheave guide. The guide may block movement of the Achilles rope in the rearward direction. The guide may extend from the rear end of the limited midfoot Achilles rope heel sheave. The guide may extend in the direction perpendicular to the heel plantar surface and away from the heel plantar surface. The guide may be at least as long as the diameter of the Achilles limit rope (e.g., ankle limit rope 414 and/or Achilles limit rope 5010). The U-shaped body of the heel rearmost surface may extend in the superior direction beyond the superior surface of the limited midfoot Achilles rope heel sheave and may function as a second guide to maintain the Achilles rope on the selected pathway.

The limited midfoot Achilles rope forefoot sheave may protrude rearward from the rearmost portion of the weight stability platform (e.g., weight stability platform 1014) of the forefoot (e.g., forefoot 12 and/or forefoot 1000). The limited midfoot Achilles rope forefoot sheave may be centered substantially on the midline of the forefoot. The limited midfoot Achilles rope forefoot sheave may be at least long enough to accommodate the specified number of wraps of the midfoot Achilles rope. The inferior face of the limited midfoot Achilles rope forefoot sheave may be shaped like substantially a second half longitudinal cylindrical surface. The second cylindrical surface may have a second axis that may be parallel to the long axis of the forefoot.

The limited midfoot Achilles rope forefoot sheave (e.g., midfoot Achilles rope forefoot sheave 1036) may include a limited midfoot Achilles forefoot sheave guide (e.g., midfoot Achilles rope forefoot sheave guide 1038). The guide may block movement of the Achilles rope in the rearward direction. The guide may be configured to not interfere with the limited midfoot Achilles rope heel sheave. The guide may extend from the rear end of the limited midfoot Achilles rope forefoot sheave. The guide may extend in the direction perpendicular to the heel plantar surface and towards the heel plantar surface. The guide may be at least as long as the diameter of the Achilles rope. The limited midfoot Achilles forefoot sheave guide may be long enough to contact a surface below the forefoot upon which the joint bears weight. The guide may extend more superior than the inferior surface of the limited midfoot Achilles rope heel sheave. The guide may be sufficiently more rearward than the rearward most end of the limited midfoot Achilles rope heel sheave to not contact the limited midfoot Achilles rope heel sheave.

The heel may include a U-shaped main body. The limited midfoot Achilles rope heel sheave may protrude rearward from the rearmost part of the U-shaped heel body. The limited midfoot Achilles rope heel sheave may be centered substantially on the midline of the heel. The limited midfoot Achilles rope heel sheave may be at least long enough to accommodate the specified number of wraps of the midfoot Achilles rope. The superior face of the limited midfoot Achilles rope heel sheave may be shaped like substantially a first half longitudinal cylindrical surface. The first cylindrical surface may have a first axis that may be parallel to the long axis of the forefoot.

The limited midfoot Achilles rope heel sheave (e.g., limited twist midfoot Achilles rope heel sheave 2020) may include a limited midfoot Achilles heel sheave guide. The guide may block movement of the Achilles rope in the rearward direction. The guide may extend from the rear end of the limited midfoot Achilles rope heel sheave. The guide may extend in the direction perpendicular to the heel plantar surface and away from the heel plantar surface. The guide may be at least as long as the diameter of the Achilles rope. The U-shaped body of the heel rearmost surface may extend in the superior direction beyond the superior surface of the limited midfoot Achilles rope heel sheave and may function as a second guide to maintain the Achilles rope on the selected pathway.

The limited midfoot Achilles rope forefoot sheave (e.g., midfoot Achilles rope forefoot sheave 1036) may protrude rearward from the rearmost portion of the midfoot attachment portion (e.g., midfoot joint attachment 168 and/or midfoot attachment portion 1004) of the forefoot. The limited midfoot Achilles rope forefoot sheave may be centered substantially on the midline of the forefoot. The limited midfoot Achilles rope forefoot sheave may be at least long enough to accommodate the specified number of may wrap of the midfoot Achilles rope. The inferior face of the limited midfoot Achilles rope forefoot sheave may be shaped like substantially a second half longitudinal cylindrical surface. The second cylindrical surface may have a second axis that may be parallel to the long axis of the forefoot.

The limited midfoot Achilles rope forefoot sheave (e.g., midfoot Achilles rope forefoot sheave 1036) may include a limited midfoot Achilles forefoot sheave guide. The guide may block movement of the Achilles rope in the rearward direction. The guide may be configured to not interfere with the limited midfoot Achilles rope heel sheave. The guide may extend from the rear end of the limited midfoot Achilles rope forefoot sheave. The guide may extend in the direction perpendicular to the heel plantar surface and towards the heel plantar surface. The guide may be at least as long as the diameter of the Achilles rope. A line tangent to the first and the second cylindrical surface may be perpendicular to the forefoot plantar surface. The Achilles rope may be disposed around the first half longitudinal cylindrical surface of the limited midfoot Achilles rope heel sheave, to and around the second half longitudinal cylindrical surface of the limited midfoot Achilles rope forefoot sheave and back to the first half longitudinal cylindrical surface. The Achilles twist rope may be disposed around the first and second half longitudinal cylindrical surface for selected number of wraps. The Achilles rope may be attached by at least one of the attachment mechanisms to a component selected from the group consisting of: the limited midfoot Achilles rope forefoot sheave, the limited midfoot Achilles rope heel sheave, and itself.

The invention may include a plantar rope complex. The plantar rope complex may include two plantar rope heel sheaves (e.g., limited twist midfoot plantar rope heel sheave 2026), plantar rope heel sheave guide holes (e.g., limited twist midfoot plantar rope heel sheave guide holes 2030), a plantar rope forefoot sheave sheaves (e.g., limited twist midfoot plantar rope forefoot sheave 1048), and a plantar rope forefoot sheave guide sheaves (e.g., limited twist midfoot plantar rope forefoot sheave guide 1049). The heel may include a U-shaped body. The plantar rope complex may be configured to allow the plantar rope (e.g., flexion limit rope 40, plantarflexion limit rope 54, and/or MTP plantarflexion limit rope 5012) to substantially block motion away from each other of the plantar rope heel sheaves and the plantar rope forefoot sheave. The motion away from each other may be substantially blocked may be along the axis parallel to the long axis of the forefoot when pulled taut and to connect the heel and the forefoot with the plantar rope.

Each midfoot plantar rope heel sheave (e.g., limited twist midfoot plantar rope heel sheave 2026) may extend from each side of the inferior surface of the heel u-shaped body in the plantar direction. Each midfoot plantar rope heel sheave may be shaped like substantially three quarters of a first longitudinal cylindrical surface. The first cylindrical surface may have a first axis substantially perpendicular to the plantar surface of the forefoot. Each plantar rope heel sheave may be on either side of the midline of the forefoot. Each plantar rope heel sheave may be far enough from the midline of the forefoot to allow a portion of the forefoot to be disposed between the two plantar rope heel sheaves. The plantar rope heel sheaves may be the most inferior sheaves of the heel. Substantially three quarters of a first longitudinal cylindrical surface may face towards one of the medial-rear, lateral-rear, and lateral-front directions. Each plantar rope heel sheave may be long enough to accommodate the selected number of may wrap of the plantar rope. Each plantar rope heel sheave may be short enough to not contact a substantially flat surface below the heel upon which the joint bears weight.

The invention may include two midfoot plantar rope heel sheave guides (e.g., limited twist midfoot plantar rope heel sheave guides 2028). Each midfoot plantar rope heel sheave guide may prevent the plantar rope (e.g., flexion limit rope 40, plantarflexion limit rope 54, and/or MTP plantarflexion limit rope 5012) from slipping off the inferior edge of the midfoot plantar rope sheave. The plantar rope heel sheave guides may extend radially from the first three quarters of a first longitudinal cylindrical surface at the inferior end of the first surface. The plantar rope heel sheave guides may extend from the planter rope, for example, about the diameter of the plantar rope.

The midfoot plantar rope forefoot sheave (limited twist midfoot plantar rope forefoot sheave 1048) may extend from the forefoot midfoot attachment portion (e.g., midfoot joint attachment 168 and/or midfoot attachment portion 1004)in the plantar direction. The midfoot plantar rope forefoot sheave may include second and third surfaces each shaped like about one quarter of a second longitudinal cylindrical surface. The second and third cylindrical surfaces may have a second and third axis, respectively, substantially perpendicular to the plantar surface of the forefoot. The plantar rope forefoot sheave third and second surfaces may be substantially equidistant from the midline of the forefoot. The second and third longitudinal cylindrical surfaces may face towards the lateral front directions of the forefoot. A line parallel to the plantar surface of the forefoot and parallel to the midline of the forefoot that may be tangent to the medial edge of either of the first cylindrical surfaces of the midfoot plantar rope heel sheaves and may be also tangent to the corresponding lateral edge of the midfoot plantar rope forefoot sheave third or second cylindrical surface. The plantar rope forefoot sheave may be the most inferior sheave of the forefoot. The plantar rope forefoot sheave may be long enough to accommodate the selected number of wraps of the plantar rope.

The midfoot plantar rope forefoot sheave may be flat on the front side between the third and fourth cylindrical surface. Each plantar rope forefoot sheave may be short enough to not contact an substantially flat surface below the forefoot on which the joint bears weight.

The invention may include a midfoot plantar rope forefoot sheave guide (limited twist midfoot plantar rope forefoot sheave guide 1049). The midfoot plantar rope forefoot sheave guide may prevent the plantarflexion limit rope (e.g., flexion limit rope 40, plantarflexion limit rope 54, and/or MTP plantarflexion limit rope 5012) from slipping off the inferior edge of the midfoot plantar rope sheave. The plantar rope forefoot sheave guides may extend radially from the first three quarters of a first longitudinal cylindrical surface at the inferior end of the first surface. The plantar rope forefoot sheave guides may extend at least about the diameter of the plantar rope.

The invention may include a midfoot plantar rope. The midfoot plantar rope 54, 5006 may limit the dorsiflexion of the midfoot joint.

The invention may include an Achilles rope (e.g., ankle limit rope 414 and/or Achilles limit rope 5010). The Achilles rope may limit the dorsiflexion of the midfoot joint.

The invention may include a heel (e.g., heel 10 and/or heel 2000). The heel may include one or more of a U shaped body 2002, a top plate 2004, plantar rope guide holes, heel strike foam supports 2010, and a forefoot clearance chamfer 2008. The U shaped body may be a piece of matter that can be disposed on either side of the midfoot attachment portion of the forefoot. The top plate may be a piece of matter that connects to the U shaped body, on the superior surface and may provide a place to mount the pylon. This may be useful for attaching to a prosthetic socket, which may be useful for attaching to a residual limb, for example, a stump of an amputee. The plantar rope guide holes may function as rope keepers, for example, ensuring that the plantar rope stays on a designated path. Heel strike foam supports may provide a place for heel strike to occur. They can have foam mounted on them, so that the forces of heel strike are attenuated by the foam. The Forefoot Clearance Chamfer may accommodate for the curved surface of the midfoot attachment portion of the forefoot. It may be a cut into the inner, inferior edge and surface of the U shaped body.

The invention may include MTP plantarflexion rope sheaves (e.g., MTP MTP plantarflexion limit rope sheave 1024). The two MTP plantarflexion rope sheaves may be shaped substantially similarly to the first surfaces of about one quarter of a first and second cylinder. The first and second cylinders may each have a first and second axis substantially perpendicular to the long axis of the forefoot (e.g., forefoot 12 and/or forefoot 1000) and substantially perpendicular to the plantar surface of the forefoot (e.g., substantially perpendicular to the superior surface of the forefoot body 1002). The first and second axes may be substantially perpendicular to the pathway of the plantarflexion rope, where the first cylindrical surface may be on the lateral and rear facing side. The MTP plantarflexion rope sheaves may protrude in the direction substantially perpendicular to the plantar surface of the forefoot (e.g., the direction substantially perpendicular to the superior/dorsal surface of the forefoot body 1002). The two MTP plantarflexion rope sheaves may be mirror images of each other, reflected around the midsagital plane of the forefoot.

The midfoot attachment portion (e.g., midfoot joint attachment 168 and/or midfoot attachment portion 1004) of the forefoot may include a MTP plantarflexion rope hole (e.g. MTP plantarflexion limit rope hole 1026). The hole may be located substantially tangent to a line parallel to the first and second axes of the cylindrically shaped first and second surfaces of the MTP plantarflexion rope sheave. The plantarflexion rope hole may be directly adjacent to the MTP plantarflexion rope sheave. Any of the aforementioned aspects may be true for the MTP plantarflexion rope hole 1026 and MTP plantarflexion rope sheaves.

The invention may include MTP axial rope forefoot sheaves (e.g., MTP axial rope forefoot sheaves 1028). The MTP axial rope forefoot sheaves may have a substantially flat configuration, from example, from flattening. Each MTP axial rope forefoot sheave 108 may include a third and a fourth surface each shaped substantially similar to the surfaces of two substantially longitudinal quarter cylinders. The third and fourth cylindrical surfaces may have third and fourth axes parallel to each other and perpendicular to the plantar surface of the forefoot. The third and fourth surfaces may be separated some distance from each other. The third and fourth surfaces may be arranged so that a line about tangent to the surface of the third cylindrical surfaces may also be substantially tangent to the surface of the fourth cylindrical surface. The two MTP axial forefoot sheaves may each be located on one of the two lateral sides of the forefoot. Each MTP axial forefoot sheave may be positioned so that one edge of the third or fourth substantially quarter cylindrically shaped surface is directly adjacent to and substantially tangent to the rearmost edge of the MTP axial rope forefoot hole (e.g., MTP axial rope forefoot hole 1020). Each MTP axial rope hole forefoot sheave may be positioned so that the other paired substantially quarter cylindrically shaped surface is directly adjacent to and substantially tangent to the frontmost edge of the MTP axial rope return hole (e.g., MTP axial rope return hole 1030).

In various embodiment, the invention may include any artificial foot and/or leg portion set forth herein including a cosmetic covering. For example, the artificial foot, leg, knee, and/or ankle may be skin colored, may have a covering that is skin-colored, may have a covering that has a skin-like texture, and/or may have coloring that has a skin-like appearance. The color may be any color, for example, any skin color or non-skin color.

The diameter of any of the sheaves set forth herein may be dictated by one or more of the diameter of the rope used, the ratio of the two diameters, and/or the acceptable strength efficiency decline associated with this ratio.

One possible advantage for the invention disclosed in this application is that it may be repairable, and that may help keep costs down in the long run. It may work like the human anatomy and may make amputees more mobile than ever possible. The technology described herein may also be applied to the Department of Defense, as well as the Department of Transportation.

At least a partial parts list of some of the embodiments set forth herein is set forth below. This list does not limit each listed part to its corresponding reference number in the drawings, and instead it is merely exemplary.

| Part | No. | Joint |
|---|---|---|
| compression member | 1 | |
| Sheaves | 2 | |
| universal rope | 3 | |
| stabilization rope | 4 | |
| limit rope | 5 | |
| tightener rope | 6 | |
| Basic Tensegrity Universal Joint | 7 | |
| heel | 10 | |
| forefoot | 12 | |
| Big Toe | 14 | |
| middle toe | 16 | |
| little toe | 20 | |
| midfoot joint ropes | 22 | Midfoot joint |
| axial rope | 28 | MTP joint |
| toe ext rope 1-1 | 30 | |
| midfoot tightener rope | 32 | Midfoot joint |
| midfoot actuators/springs | 34 | Midfoot joint |
| toe actuators | 36 | MTP joint |
| extension limit ropes | 38 | MTP joint |
| flexion limit ropes | 40 | MTP joint |
| screw swages | 42 | MTP joint |
| stop swages | 44 | MTP joint |
| top swage keeper | 46 | MTP joint |
| bottom rope keeper | 48 | MTP joint |
| rope guide keepers | 50 | MTP joint |
| twist rope | 52 | midfoot twist |
| plantarflexion limit rope | 54 | midfoot twist |
| plantarflexion limit rope sheave | 56 | midfoot twist |
| plantarflexion limit rope guide | 58 | midfoot twist |
| round midfoot outer sheaves | 60 | midfoot twist |
| dorsiflexion limit rope | 62 | midfoot twist |
| dorsiflexion limit rope sheave | 64 | midfoot twist |
| dorsiflexion limit rope guide | 66 | midfoot twist |
| toe supports | 102 | forefoot |
| front sheaves | 104 | forefoot |
| axial rope hole | 106 | forefoot |
| tall axial sheave | 108 | forefoot |
| short axial sheave | 110 | forefoot |
| axial sheave rope keeper flange | 111 | forefoot |
| spacer for flexion ropes | 112 | forefoot |
| extension limit rope holes | 116 | forefoot |
| flexion limit rope holes | 120 | forefoot |
| limit rope guides | 126 | forefoot |
| flexion limit rope spreader | 130 | forefoot |
| extension limit rope spreader | 136 | forefoot |
| stiffener bars | 152 | forefoot |
| medial angled walking surface | 160 | forefoot |
| lateral angled walking surface | 162 | forefoot |
| screw attachment bar | 164 | forefoot |
| dorsiflexion limit rope hole | 166 | forefoot |
| midfoot joint attachment | 168 | forefoot |
| upper midfoot rope hole | 176 | forefoot |
| lower midfoot rope hole | 178 | forefoot |
| actuator stays | 180 | forefoot |
| tightener rope hole | 182 | forefoot |
| rear weight platform | 184 | forefoot |
| axial rope screw hole attachment | 185 | forefoot |
| axial rope swage loop attachment hole | 186 | forefoot |
| alt midfoot attachment hole upper | 187 | midfoot twist |
| alt midfoot attachment hole lower | 188 | midfoot twist |
| alt plantar flexion limit rope sheave | 189 | midfoot twist |
| sheave structural support | 190 | midfoot twist |
| plantar flexion limit rope guide | 191 | midfoot twist |
| plantar flexion sheave surface | 192 | midfoot twist |
| cushioned heel | 200 | heel |
| midfoot joint outer sheaves | 202 | heel |
| midfoot outer sheave distal rope guide | 203 | heel |
| Movement Clearance | 204 | heel |
| midfoot outer sheave proximal rope guide | 205 | heel |
| pylon | 206 | heel |
| tightener rope hole | 210 | heel |
| actuator stay | 211 | heel |
| alt - tapered midfoot joint outer sheaves | 212 | heel |
| cushion cavity | 214 | heel |
| coordination rope attachment | 216 | heel |
| angled plate (mount for sheaves) | 218 | heel |
| ankle limit rope holes | 220 | heel |
| side sheave | 300 | toe |
| top sheave | 302 | toe |
| angled walking surface. | 304 | toe |
| main column | 306 | toe |
| axial rope hole | 312 | toe |
| actuator hook | 316 | toe |
| top sheave | 330 | toe |
| Bearing Clearance | 334 | toe |
| thick web | 338 | toe |
| Thin Web | 340 | toe |
| top beam | 342 | toe |
| toe race | 380 | toe bearing and |
| | | race |
| inner curve | 382 | toe bearing and |
| | | race |
| Bearing | 384 | toe bearing and |
| | | race |
| ankle universal rope | 400 | ankle |
| ankle stabilizer rope (×2) | 402 | ankle |
| heel attachment stubs | 404 | ankle |
| heel universal rope holes | 406 | ankle |
| heel stabilizer rope holes | 408 | ankle |
| lower leg universal rope holes | 410 | ankle |
| lower leg stabilizer rope holes | 412 | ankle |
| xbrace | 500 | ankle |
| sheaves | 502 | knee |
| upper joint member (thigh) | 504 | knee |
| lower joint member (lower leg - pylon) | 506 | knee |
| upper knee universal rope | 508 | knee |
| lower knee universal rope | 510 | knee |
| upper knee stabilization ropes (×4) | 512 | knee |
| lower knee stabilization ropes (×4) | 514 | knee |
| xbrace universal rope holes | 516 | knee |
| xbrace stabilization rope holes | 518 | knee |
| upper joint member universal rope holes | 520 | knee |
| lower joint member universal rope holes | 522 | knee |
| upper joint member stabilization rope holes | 524 | knee |
| lower joint member stabilization rope holes | 526 | knee |
| knee tightener rope | 528 | knee |
| xbrace tightener rope hole | 530 | knee |
| upper joint member tightener rope hole | 532 | knee |
| iower joint member tightener rope hole | 534 | knee |
| u-shaped end of upper joint member | 538 | knee |
| u-shaped end of lower joint member | 540 | knee |
| curved surface of shaped sheave | 542 | knee |
| edge of shaped sheave | 544 | knee |
| forefoot | 1000 | forefoot |
| forefoot body | 1002 | forefoot |
| midfoot attachment portion | 1004 | forefoot |
| walking surface | 1006 | forefoot |
| small foot toe supports | 1008 | forefoot |
| toe support central fillet | 1010 | forefoot |
| coordination rope void | 1012 | forefoot |
| weight stability platform | 1014 | forefoot |
| MTP dorsiflexion forefoot sheaves | 1016 | forefoot |
| MTP dorsiflexion forefoot sheave guide | 1018 | forefoot |
| MTP axial rope forefoot hole | 1020 | forefoot |
| MTP dorsflexion rope terminal screw holes | 1022 | forefoot |
| MTP plantarflexion limit rope sheave | 1024 | forefoot |
| MTP plantarflexion limit rope hole | 1026 | forefoot |
| MTP axial rope forefoot sheave | 1028 | forefoot |
| MTP axial rope return hole | 1030 | forefoot |
| MTP axial rope terminal screw attachment | 1032 | forefoot |
| cutouts | | |
| MTP axial rope terminal screw holes | 1034 | forefoot |
| midfoot Achilles rope forefoot sheave | 1036 | forefoot |
| midfoot Achilles rope forefoot sheave guide | 1038 | forefoot |
| limited twist midfoot twist rope forefoot | 1040 | forefoot |
| sheave | | |
| limited twist midfoot twist rope forefoot | 1042 | forefoot |
| sheave end fillet | | |
| limited twist midfoot twist rope forefoot | 1043 | forefoot |
| sheave cap | | |
| limited twist midfoot twist rope forefoot | 1044 | forefoot |
| top sheave | | |
| limited twist midfoot twist rope forefoot | 1045 | forefoot |
| bottom sheave | | |
| limited twist midfoot plantarflexion limit | 1046 | forefoot |
| rope forefoot sheave | | |
| limited twist midfoot plantar rope forefoot | 1048 | forefoot |
| sheave | | |
| limited twist midfoot plantar rope forefoot | 1049 | forefoot |
| sheave guide | | |
| limited twist midfoot plantar rope terminal | 1050 | forefoot |
| screw mount | | |
| limited twist midfoot plantar rope terminal | 1052 | forefoot |
| screw hole | | |
| limited twist midfoot plantar rope stop mount | 1054 | forefoot |
| limited twist midfoot plantar rope stop hole | 1056 | forefoot |
| heel | 2000 | heel |
| U-shaped body | 2002 | heel |
| top plate | 2004 | heel |
| pylon | 2006 | heel |
| forefoot clearance chamfer | 2008 | heel |
| heel strike support | 2010 | heel |
| Heel strike pad | 2011 | heel |
| limited twist midfoot twist rope heel sheave | 2012 | heel |
| limited twist midfoot twist rope heel superior | 2013 | heel |
| guide sheave | | |
| limited twist midfoot twist rope heel inferior | 2014 | heel |
| guide sheave | | |
| front edge of inferior guide sheave | 2015 | heel |
| limited twist midfoot twist rope heel inferior | 2016 | heel |
| sheave guide | | |
| limited twist midfoot twist rope superior | 2017 | heel |
| sheave | | |
| limited twist midfoot twist rope inferior | 2018 | heel |
| sheave | | |
| rear edge of superior guide sheave | 2019 | heel |
| limited twist midfoot achilles rope heel sheave | 2020 | heel |
| limited twist midfoot achilles rope heel stop | 2021 | heel |
| hole | | |
| limited twist midfoot achilles rope heel stop | 2022 | heel |
| swage keeper | | |
| limited twist midfoot achilles rope heel screw | 2024 | heel |
| hole | | |
| limited twist midfoot plantar rope heel sheave | 2026 | heel |
| limited twist midfoot plantar rope heel sheave | 2028 | heel |
| guide | | |
| limited twist midfoot plantar rope heel sheave | 2030 | heel |
| guide holes | | |
| limited twist midfoot twist rope heel terminal | 2032 | heel |
| sheave | | |
| limited twist midfoot twist rope heel terminal | 2034 | heel |
| screw hole | | |
| limited twist midfoot twist rope heel terminal | 2036 | heel |
| stop hole | | |
| toe (wide) | 3000 | toe |
| attachment hook | 3004 | toe |
| toe thick web | 3006 | toe |
| walking surface | 3008 | toe |
| MTP dorsiflexion limit rope toe sheave | 3012 | toe |
| side sheave | 3014 | toe |
| side sheave guide | 3016 | toe |
| distal sheave | 3018 | toe |
| distal sheave guide | 3020 | toe |
| MTP axial rope toe hole | 3022 | toe |
| MTP plantarflexion limit rope toe sheave | 3024 | toe |
| MTP plantarflexion limit rope toe sheave guide | 3025 | toe |
| toe thin web | 3026 | toe |
| race | 4000 | toe bearing and |
| | | race |
| curved inner surface | 4002 | toe bearing and |
| | | race |
| central ring | 4004 | toe bearing and |
| | | race |
| side bearing race | 4006 | toe bearing and |
| | | race |
| bearings | 4100 | toe bearing and |
| | | race |
| plantar rope | 5006 | foot |
| limited twist midfoot rope | 5008 | foot |
| Achilles rope | 5010 | foot |
| MTP plantarflexion limit rope | 5012 | foot |
| MTP dorsiflexion limit rope | 5014 | foot |
| MTP Axial rope | 5016 | foot |
| cross joint coordination rope | 5018 | foot |
| rope termination hardware | 5020 | foot |
| tensegrity MTP joint | 5022 | foot |
| tensegrity limited twist midfoot joint | 5024 | foot |
| limited twist midfoot plantarflexion limit rope | 5026 | foot |
| artificial foot | 7000 | |
| artificial knee | 7002 | |
| artificial MTP joint | 7003 | |
| artificial midfoot joint | 7004 | |
| artificial leg | 7005 | |
| artificial midfoot twist joint | 7006 | |
| artificial foot | 8000 | |
| artificial ankle | 8001 | |
| artificial universal tensegrity joint | 8002 | |
| artificial MTP joint | 8003 | |
| artificial midfoot joint | 8004 | |
| artificial midfoot twist joint | 8006 | |
| artificial limited twist midfoot joint | 8007 | |

Other embodiments of the invention may be apparent to those skilled in the art from consideration of the specification and practice of the invention disclosed herein. It may be intended that the specification and examples be considered as exemplary only, with a true scope and spirit of the invention being indicated by the following claims.

1. At least a portion of an artificial prosthetic or orthotic foot for a human, or a robotic foot comprising:
an artificial midfoot joint;
an artificial metatarsophalangeal (MTP) joint; and
a mechanical coupler configured to coordinate movement of the artificial midfoot joint relative to the artificial MTP joint.

2. The at least a portion of an artificial foot of claim 1, wherein the mechanical coupler is configured to move the artificial midfoot joint into plantarflexion when the artificial MTP joint moves into dorsiflexion.

3. The at least a portion of an artificial foot of claim 1, further comprising an artificial toe and an artificial forefoot;
wherein the artificial MTP joint connects the artificial toe to the artificial forefoot.

4. The at least a portion of an artificial foot of claim 1, further comprising an artificial forefoot and an artificial heel;
wherein the artificial midfoot joint connects the artificial forefoot to the artificial heel.

5. The at least a portion of an artificial foot of claim 1, wherein the coordinated movement of the artificial midfoot joint relative to the artificial MTP joint substantially corresponds to a coordinated movement of a natural midfoot joint relative to a natural MTP joint during ambulation of a natural human foot.

6. The at least a portion of an artificial foot of claim 1, further comprising an artificial toe,
wherein when a weight is removed from the artificial toe by a contralateral leg heelstrike gait event, the mechanical coupler is configured provide a spring-like action that pulls the artificial midfoot joint and the artificial MTP joint and causes them to move substantially synchronously, rotating in opposite directions,
wherein the spring-like action releases energy that propels the at least a portion of the artificial forward and into a swing phase gait event.

7. The at least a portion of an artificial foot of claim 1, further comprising an artificial toe and an artificial heel,
wherein the mechanical coupler is configured to coordinate movement of the artificial toe relative to the artificial heel.

8. The at least a portion of an artificial foot of claim 9, wherein mechanical coupler is a tension member,
wherein the tension member is configured to move the artificial toe substantially synchronously with the artificial heel when the tension member is in a taut configuration.

9. The at least a portion of an artificial foot of claim 1, wherein the at least a portion of an artificial foot is a whole artificial foot.

10. The at least a portion of an artificial foot of claim 1, wherein at least one of the artificial midfoot joint and the artificial MTP joint includes a tensegrity joint.

11. The at least a portion of an artificial foot of claim 1, wherein the artificial midfoot joint has a range of motion, from a substantially maximal excursion in a plantarflexion direction to a substantially maximal excursion in a dorsiflexion direction, between about 0.1 degrees and about 120 degrees.

12. The at least a portion of an artificial foot of claim 11, wherein the artificial midfoot joint has a range of motion, from the substantially maximal excursion in the plantarflexion direction to the substantially maximal excursion in the dorsiflexion direction, between about 0.5 degrees and about 60 degrees.

13. The at least a portion of an artificial foot of claim 12, wherein the artificial midfoot joint has a range of motion, from the substantially maximal excursion in the plantarflexion direction to the substantially maximal excursion in the dorsiflexion direction, between about 1 degree and about 30 degrees.

14. The at least a portion of an artificial foot of claim 13, wherein the artificial midfoot joint has a range of motion, from the substantially maximal excursion in the plantarflexion direction to the substantially maximal excursion in the dorsiflexion direction, between about 1 degree and about 10 degrees.

15. The at least a portion of an artificial foot of claim 1, wherein the artificial midfoot joint has a range of motion, from a substantially maximal excursion in a plantarflexion direction to a substantially maximal excursion in a dorsiflexion direction, substantially similar to a corresponding natural human subtalar joint.

16. The at least a portion of an artificial foot of claim 1, wherein the artificial midfoot joint has a range of motion, from a substantially maximal excursion in a plantarflexion direction to a substantially maximal excursion in a dorsiflexion direction, substantially similar to a sum of ranges of motion of a human ankle and human subtalar joints.

17. The at least a portion of an artificial foot of claim 1, wherein the artificial MTP joint has a range of motion, from a substantially maximal excursion in a plantarflexion direction to a substantially maximal excursion in a dorsiflexion direction, between about 0.1 degrees and about 340 degrees.

18. The at least a portion of an artificial foot of claim 17, wherein the artificial MTP joint has a range of motion, from the substantially maximal excursion in the plantarflexion direction to the substantially maximal excursion in the dorsiflexion direction, between about 0.5 degrees and about 60 degrees.

19. The at least a portion of an artificial foot of claim 18, wherein the artificial MTP joint has a range of motion, from the substantially maximal excursion in the plantarflexion direction to the substantially maximal excursion in the dorsiflexion direction, between about 1 degree and about 30 degrees.

20. The at least a portion of an artificial foot of claim 19, wherein the artificial MTP joint has a range of motion, from the substantially maximal excursion in the plantarflexion direction to the substantially maximal excursion in the dorsiflexion direction, between about 1 degree and about 15 degrees.

21. The at least a portion of an artificial foot of claim 1, wherein the artificial MTP joint has a range of motion, from a substantially maximal excursion in a plantarflexion direction to a substantially maximal excursion in a dorsiflexion direction, substantially similar to a corresponding natural human MTP joint.

22. The at least a portion of an artificial foot of claim 1, wherein the mechanical coupler has elastic properties.

23. The at least a portion of an artificial foot of claim 1, wherein the mechanical coupler is configured to store energy.

24. The at least a portion of an artificial foot of claim 1, wherein the mechanical coupler is configured to release stored energy when the artificial midfoot joint and the artificial MTP joint move substantially synchronously, rotating in opposite directions.

25. The at least a portion of an artificial foot of claim 1, wherein the mechanical coupler is configured to compliantly couple the movement of the artificial MTP joint relative to the artificial midfoot joint.

26. The at least a portion of an artificial foot of claim 1, wherein the mechanical coupler is configured such that after the mechanical coupler is pulled taut, the mechanical coupler is configured to allow for an input of energy to one or more portions of the artificial foot as the artificial MTP joint and the artificial midfoot joint and causes them to move substantially synchronously, rotating in opposite directions

27. The at least a portion of an artificial foot of claim 20, wherein the artificial MTP joint has a range of motion, from the substantially maximal excursion in the plantarflexion direction to the substantially maximal excursion in the dorsiflexion direction, between about 1 degree and about 10 degrees.

28. The at least a portion of an artificial foot of claim 27, wherein the artificial MTP joint has a range of motion, from the substantially maximal excursion in the plantarflexion direction to the substantially maximal excursion in the dorsiflexion direction, between about 1 degree and about 5 degrees.
